(19) United States
(12) Patent Application Publication
(10) Pub. No.: US 2025/0304968 A1
(43) Pub. Date: Oct. 2, 2025

(54) UBE3A ANTISENSE THERAPEUTICS

(71) Applicant: Quiver Holdings Inc., Cambridge, MA (US)

(72) Inventors: James Fink, Cambridge, MA (US); Luis Williams, Cambridge, MA (US); Caitlin Lewarch, Cambridge, MA (US); David Gerber, Somerville, MA (US); Duncan Brown, Cambridge, MA (US); Sudhir Agrawal, Cambridge, MA (US); Graham T. Dempsey, Sudbury, MA (US)

(21) Appl. No.: 19/188,812

(22) Filed: Apr. 24, 2025

Related U.S. Application Data
(63) Continuation-in-part of application No. 17/674,692, filed on Feb. 17, 2022.
(60) Provisional application No. 63/150,188, filed on Feb. 17, 2021.

(51) Int. Cl. C12N 15/113

(52) U.S. Cl. CPC C12N 15/1137; C12N 2310/11; C12N 2310/313; C12N 2310/321

(57) ABSTRACT

The invention provides compositions useful to knock down overexpression of UBE3A and treat conditions associated with Dup15q syndrome. The compositions include antisense oligonucleotides, preferably short oligonucleotides that are complementary to, and hybridize to, UBE3A transcripts in vivo. The ASOs prevent or inhibit successful translation of UBE3A mRNA into protein. Specifically, preferred embodiments include anti-UBE3A gapmers—oligos that include a central DNA portion flanked by RNA wings. When the gapmer hybridizes to UBE3A pre-mRNA or mRNA, the duplex hybrid recruits RNaseH, which cleaves, or digests, the UBE3A pre-mRNA or mRNA, preventing expression of the UBE3A protein. Because the ASOs prevent expression of the UBE3A protein, treatment with a composition including ASOs of the disclosure may be effective to knock down overexpression of UBE3A.

### TECHNICAL FIELD

The disclosure relates to treatments for neurological disorders.

### SEQUENCE LISTING

A “Sequence Listing XML” is submitted herewith in XML file format and (i) the name of the file is qsta36.xml; (ii) the date of creation is Apr. 24, 2025; and (iii) the size of the file is 293,031 bytes and the material in the XML file is incorporated by reference.

### BACKGROUND

Ubiquitin ligase proteins, such as the E3 ligase E6-associated protein (E6AP, also known as UBE3A), are implicated in neurological and neurodevelopmental disorders. For example, E6AP is encoded by the UBE3A gene and expression of the UBE3A gene is regulated via genetic imprinting. Loss of E6AP expression leads to the development of Angelman syndrome, typically characterized by impaired speech and motor development, as well as seizures. Conversely, copy number variations (CNVs) of UBE3A may be linked to overexpression of E6AP and consequent development of autism spectrum disorders (ASDs).

In some clinical presentations, a portion of chromosome 15 is duplicated. This Dup15q syndrome most commonly occurs in one of two forms, an extra isodicentric chromosome 15 or an interstitial duplication in chromosome 15. Dup15q syndrome is characterized by hypotonia and gross and fine motor delays, intellectual disability, autism spectrum disorder (ASD), and epilepsy, including infantile spasms. It is thought that increased copy number for methylated maternal 15q duplications leads to increased protein expression and that overexpression of UBE3A is linked to severity in Dup15q, where the increased number of maternal alleles is thought to be the primary driver of Dup15q pathology.

### SUMMARY

The invention provides compositions for treating disorders associated with CNVs of the UBE3A gene. Specifically, the disclosure provides antisense oligonucleotides useful to knock down overexpression of UBE3A for treatment of seizures, hypotonia, motor delays, intellectual disability, disorders presenting seizures, and autism spectrum disorders (ASD) that arise in subjects affected by Dup15q syndrome. Compositions of the invention include antisense oligonucleotides that are complementary to, and hybridize to, UBE3A transcripts in vivo. The ASOs prevent translation of UBE3A mRNA into protein. Specifically, preferred embodiments include anti-UBE3A gapmers-oligos that include a central DNA portion flanked by RNA wings. When the gapmer hybridizes to UBE3A pre-mRNA or mRNA, the hybrid duplex recruits RNaseH, which cleaves, or digests, the UBE3A pre-mRNA or mRNA, preventing expression of the UBE3A protein. Because the ASOs prevent expression of the UBE3A protein, treatment with a composition including ASOs of the disclosure is effective to knock down overexpression of UBE3A. Accordingly, compositions of the disclosure are useful to treat Dup15q syndrome and its symptoms.

Oligonucleotides of the disclosure are designed to bind to certain targets in the RNA s used in synthesis of ubiquitin ligase proteins. Binding of the oligonucleotides prevents protein synthesis and downregulates expression of the ubiquitin ligase. Specifically, oligonucleotides of the invention have a sequence that is substantially or entirely complementary to one of the identified targets on a ubiquitin protein ligase E3A pre-mRNA or mRNA. That is, the oligonucleotides are antisense to the identified target. When the antisense oligonucleotide (ASO) hybridizes to its target RNA, it forms a double-stranded ASO:RNA duplex that recruits an enzyme (RNaseH) that degrades a portion of the double-stranded duplex. Degrading the ASO:RNA duplex depletes the cell of E6AP mRNA, which decreases the amount of E6AP synthesized by the cell.

Thus, when a composition that includes oligonucleotides that are antisense to the identified targets in E6AP pre-mRNA or mRNA is administered to a patient, the composition will decrease expression of E6AP that may otherwise result from copy number variations of UBE3A or the chromosome 15q11.2-q13.1 duplication syndrome known as Dup15q syndrome.

In certain aspects, the disclosure provides compositions for treating Dup15q. Such compositions include a synthetic antisense oligonucleotide (ASO) that inhibits expression of a ubiquitin ligase protein. Preferably, the protein is ubiquitin protein ligase E3A. The ASO hybridizes to a complementary target in a transcript from a UBE3A gene. The sequence of bases in the ASO may have at least 80% identity to one of SEQ ID NOS: 1-228, preferably one of SEQ ID NOS: 1-40, and more preferably one of SEQ ID NOS: 146, 155, 156, 158, 159, 161, 164 169, 174, 175, 178, 179, 213, and 214. In some embodiments, a sequence of bases in the ASO is at least 90%, 95%, or 100% identical to one of SEQ ID NOS: 1-228, 1-40, or 146, 155, 156, 158, 159, 161, 164 169, 174, 175, 178, 179, 213, and 214, and the oligonucleotide can hybridize to, and induce RNase cleavage of, UBE3A pre-mRNA or mRNA.

In some embodiments, the oligonucleotide comprises two RNA wings flanking a central region of at least 10 DNA bases, preferably about 12 bases. At least one of the two wings of the ASO comprises modified RNA bases. Each modified RNA base may be selected from the group consisting of 2′-O-methoxyethyl RNA and 2′-O-methyl RNA. The ASO may include at least about 20 bases, preferably between about 15 about 25 bases. In certain embodiments, the ASO has a backbone comprising a plurality of phosphorothioate bonds. The ASOs provided herein include a central region of 10-12 bases and flanking regions of 4-5 bases.

A preferred ASO has a base sequence that has been screened and determined to not meet a threshold match for any non-target transcripts in humans. Optionally the ASO has a base sequence with 0 mismatches to a homologous segment in a non-human primate genome and no more than about 5 mismatches in a homologous segment in a rodent genome.

In certain embodiments, a composition of the invention comprises a plurality of ASOs, each having a base sequence at least about 80% identical to one of SEQ ID NOS: 1-228, wherein each of the ASOs has a gapmer structure that comprises a central DNA segment flanked by RNA wings. In certain preferred embodiments, the composition comprises a plurality of ASOs each having a base sequence at least about 80% identical to one of SEQ ID NOS: 1-40, and more preferably to one of SEQ ID NOS: 146, 155, 156, 158, 159, 161, 164, 169, 174, 175, 178, 179, 213, and 214, wherein each of the ASOs has a gapmer structure that comprises a central DNA segment flanked by RNA wings. Each oligonucleotide may have a base sequence with at least about a90% (or 95%, or 100%) match to one of SEQ ID NO: 1-228 (preferably 1-40 and more preferably 146, 155, 156, 158, 159, 161, 164, 169, 174, 175, 178, 179, 213, and 214), with bases linked only by phosphorothioate linkages, the oligonucleotide further comprising a central 10 DNA bases flanked by a 5′ wing and a 3′ wing, the 5′ wing and the 3′ wing each comprising five consecutive 2′ modified RNA bases.

In some embodiments, each oligonucleotide has a base sequence matching one of SE Q ID NO: 1-228, with at least a majority of inter-base linkages comprising phosphorothioate linkages, the oligonucleotide further comprising a central 10 DNA bases flanked by a 5′ wing and a 3′ wing, the 5′ wing and the 3′ wing each comprising five consecutive 2′-O-methoxyethyl (2′-MOE) 2′-MOE RNA bases. In preferred embodiments, each oligonucleotide has a base sequence matching one of SEQ ID NO: 1-40, with at least a majority of inter-base linkages comprising phosphorothioate linkages, the oligonucleotide further comprising a central 10 DNA bases flanked by a 5′ wing and a 3′ wing, the 5′ wing and the 3′ wing each comprising five consecutive 2′ MOE RNA bases. In more preferred embodiments, each oligonucleotide has a base sequence matching one of SEQ ID NO: 146, 155, 156, 158, 159, 161, 164, 169, 174, 175, 178, 179, 213, and 214, with at least a majority of inter-base linkages comprising phosphorothioate linkages, the oligonucleotide further comprising a central 10 DNA bases flanked by a 5′ wing and a 3′ wing, the 5′ wing and the 3′ wing each comprising five consecutive 2′ MOE RNA bases.

In related aspects, the invention provides methods for treating Dup15q syndrome, which methods include delivering one of the disclosed compositions to a subject in need thereof, e.g., to downregulate overexpression of UBE3A. Therapeutic oligonucleotides of the disclosure may have a gapmer structure that includes a central DNA segment flanked by modified RNA wings. Such a therapeutic oligonucleotide may include two wings flanking a central region of DNA bases (e.g., about 10 to 14 DNA bases, e.g., central region of about 12 DNA bases). Preferably at least one end of the oligonucleotide comprises modified RNA bases, e.g., any number or any combination of 2′-O-methoxyethyl RNA (“2′-MOE”) and/or 2′-O-methyl RNA (“2′ O-Me”). In addition, compositions of the invention may be designed to target an exon-exon junction to differentially target cytoplasmic mRNA versus nuclear pre-mRNA. Thus, ASOs of the invention can be designed to interact with RNA prior to or after splicing, adding specificity and versatility to the compositions.

In various embodiments, therapeutic oligonucleotide may be provided in a solution or carrier formulated for delivery via any suitable route including, for example, intravenously or intrathecally. The oligonucleotide may be of any suitable length, e.g., at least about 18 bases, and preferably between about 15 and about 25 bases. The oligonucleotide may have phosphorothioate bonds in its backbone. In preferred embodiments, the oligonucleotide has a base sequence that has been screened and determined to not meet a threshold match for any long, non-coding RNA or other off-target sequences or transcripts in humans. The oligonucleotide may have a base sequence with 0 mismatches to a homologous segment in a non-human primate genome and no more than about 5 mismatches in a homologous segment in a rodent genome.

When the composition is delivered to cells in vitro, the cells exhibit a dose-dependent knockdown of UBE3A. The oligonucleotide may be a gapmer having a base sequence with at least about a 90% match to one of SEQ ID NO: 1-228, with at least some phosphorothioate linkages. The linkages may be all phosphorothioate or a mixture of phosphorothioate and phosphodiester bonds. The oligonucleotide may further have a central 12 DNA bases flanked by a 5′ wing and a 3′ wing, the 5′ wing and the 3′ wing each comprising four consecutive 2′ modified RNA bases. Preferably, the oligonucleotide has a base sequence matching one of SE Q ID NO: 1-228, with bases linked by phosphorothioate linkages, and a structure having central DNA bases flanked by a 5′ wing and a 3′ wing. The number of RNA bases in the wings and DNA bases in the central segment may be 5-10-5 or 4-12-4, or a similar suitable pattern. The 5′ wing and the 3′ wing may each include several 2′-MOE RNA bases. For example, the oligonucleotide may have 4 consecutive 2′-MOE RNA bases in each wing with a central 12 DNA bases (a “4-12-4” structure), with phosphorothioate linkages throughout the central DNA segment and a mixture of phosphorothioate and phosphodiester bonds in the wings.

Alternatively, the oligonucleotide may have 5 consecutive 2′-MOE RNA bases in each wing with a central 10 DNA bases (a “5-10-5” structure), with phosphorothioate linkages throughout the central DNA segment and a mixture of phosphorothioate and phosphodiester bonds in the wings. The 5′ and 3′ wings could also be of different length in the same ASO, e.g., a “4-11-5” or a “5-11-4” structure.

In combination embodiments, the invention provides compositions that include a plurality of copies of a plurality of distinct therapeutic gapmers, each according to the descriptions above, in a suitable formulation or carrier.

Aspects of the disclosure relate to use of an antisense oligonucleotide (ASO) for the manufacture of a medicament for treating Dup15q syndrome. In such embodiments, the ASO has at least about 75% identity with one of SEQ ID NOS: 1-228, and more preferably at least about 90% identity, e.g., 95% or 100% identity. Preferred embodiments use an ASO that is between about 15 and 25 bases in length, preferably between about 18 and 22, or between about 19 and 21 (inclusive). In general, reference to “an ASO” includes numerous copies of substantially identical molecules. Accordingly, “an ASO” may be any number, e.g., hundreds of thousands, or millions, of copies of the indicated ASO. In preferred embodiments, the ASO is 20 bases in length and has the sequence of one of SEQ ID NOS: 1-228 and is used in the manufacture of a medicament for the treatment of Dup15q syndrome. The ASO may be provided in any suitable format such as, for example, lyophilized in a tube or in solution in a tube, such as a microcentrifuge tube or a test tube. Preferred embodiments of the use target transcripts of the UBE3A gene. One or more (e.g., two, three, four, or five, or more) ASOs may be used in manufacture of the medicament. The one or more ASOs may hybridize to a target in the UBE3A pre-mRNA or mRNA. In certain embodiments, a sequence of bases in the ASO is at least about 90% identical to one of SEQ ID NOS: 1-228. In other embodiments, the ASO may have a gapmer structure with a central DNA segment flanked by RNA wings, e.g., a central region of 12 DNA bases with 4 modified RNA bases on both sides of the central region. Each modified RNA base may be 2′-MOE. Preferably a backbone of the ASO has a plurality of phosphorothioate bonds. Accordingly, the ASO may initially be in a form suitable for mixing into a formulation suitable for introduction by injection or a pump. For example, the ASO (thousands or millions or more of copies of one ASO) may be lyophilized in a tube or in solution at a known quantity, molality, or concentration. The ASO may be dissolved or diluted into a pharmaceutically acceptable composition in which a carrier, such as a solvent and/or excipient, includes the ASO and may be loaded in an IV bag, syringe, or pump. The medicament may be made using more than one ASO, e.g., any combination of 2, 3, 4, or 5, or more. Bases in compositions of the invention may be modified or wobble bases, which may be used in order to increase the breadth and effectiveness of compositions of the invention. In one example, ASOs for use in the invention may contain methylated bases (e.g., 5-methylcytosine, 5-methyluracil (thymine) and others).

Compositions of the invention may be formulated to accommodate serial dosing. For example, formulations may provide dosages to be administered at two or more separate times and, optionally, with two or more different ASOs, in order to take advantage of optimal therapeutic windows and to avoid potential competition between ASOs. In addition, compositions of the invention, whether administered serially or not, may interact with more than one target, depending on the composition of the ASOs involved. For example, ASOs may comprise targeted mismatches that allow interaction with multiple targets (both within and across mRNA and pre-mRNA species), thus allowing the associated treatment to impact transcripts from more than one gene copy. Compositions of the invention may also be delivered in a time-release format and/or comprising adjuvants to increase serum half-life.

### BRIEF DESCRIPTION OF THE DRAWINGS

FIG. 1 shows a composition for treating Dup15q Syndrome.

FIG. 2 shows an oligonucleotide (ASO) with a gapmer structure.

FIG. 3 shows results from screening 40 UBE3A exonic ASOs.

FIG. 4 gives results showing dose-response of ten ASO candidates.

FIG. 5 shows results from screening human exonic ASOs with mouse homology.

FIG. 6 shows a table summarizing qPCR readouts of UBE3A knockdown, expressed as percent of UBE3A knockdown, for certain screened ASOs of the invention.

FIG. 7 shows a table summarizing qPCR readouts of UBE3A knockdown, expressed as percent of UBE3A knockdown, for certain screened ASOs of the invention.

FIG. 8 shows a table summarizing qPCR readouts of UBE3A knockdown, expressed as percent of UBE3A knockdown, for certain screened ASOs of the invention.

FIG. 9 shows a table summarizing qPCR readouts of UBE3A knockdown, expressed as percent of UBE3A knockdown, for certain screened ASOs of the invention.

FIG. 10 shows UBE3A ASO dose-response modulation of target expression for 2 lead candidate example ASOs and their PO-modified daughter molecules in Dup15q patient fibroblasts (top) or mouse embryonic fibroblasts (bottom).

FIG. 11 shows plots of the dose-response and indicates EC50 for the same 2 example lead candidate ASOs from FIG. 10.

FIG. 12 shows dose-response data for lead all-PS backbone ASO candidates of the invention that target UBE3A exons.

FIG. 13 shows dose-response data for lead all-PS backbone ASO candidates of the invention that target UBE3A introns.

FIG. 14 shows dose-response data for lead all-PS backbone ASO candidates of the invention that have 100% mouse homology for rodent in vivo efficacy studies.

FIG. 15 shows dose-response data for PO-modified daughter lead ASO candidates that have 100% mouse homology for rodent in vivo efficacy studies.

FIG. 16 shows dose-response data for PO-modified daughter lead ASO candidates of the invention for human clinical candidate studies.

FIG. 17 shows a western blot for a certain candidate lead UBE3A ASO and 3 PO-modified daughter molecules with identical ASO sequences.

FIG. 18 show a quantification of the UBE3A protein knockdown for the ASOs of FIG. 17.

FIG. 19 provides a table summarizing UBE3A protein knockdown results for lead all-PS backbone ASO candidates targeting UBE3A.

FIG. 20 provides a table summarizing UBE3A protein knockdown results for lead all-PS backbone ASO candidates with 100% mouse homology for rodent in vivo efficacy studies.

FIG. 21 provides a table summarizing UBE3A protein knockdown results for PO-modified daughter lead ASO candidates with 100% mouse homology for rodent in vivo efficacy studies.

FIG. 22 provides a table summarizing UBE3A protein knockdown results for PO-modified daughter lead ASO candidates for human clinical candidates.

FIG. 23 provides data showing the knockdown of UBE3A transcript in human NGN2 stem cell-derived neurons using UBE3A lead candidate ASOs of the invention.

FIG. 24 provides data showing the knockdown of UBE3A transcript in human primary neurons using UBE3A lead candidate ASOs of the invention.

FIG. 25 provides data showing the knockdown of UBE3A transcript in non-human primate primary fibroblast cultures using UBE3A lead ASO candidates of the invention.

FIG. 26 provides data showing the knockdown of UBE3A transcript in mouse primary cortical neurons using UBE3A lead candidate ASOs of the invention.

FIG. 27 provides data showing the knockdown of UBE3A transcript in rat primary cortical neurons using UBE3A lead ASO candidates of the invention where the cells were harvested for qPCR after four days.

FIG. 28 provides data showing the knockdown of UBE3A transcript in rat primary cortical neurons using UBE3A lead ASO candidates of the invention where the cells were harvested for qPCR after eight days.

### DETAILED DESCRIPTION

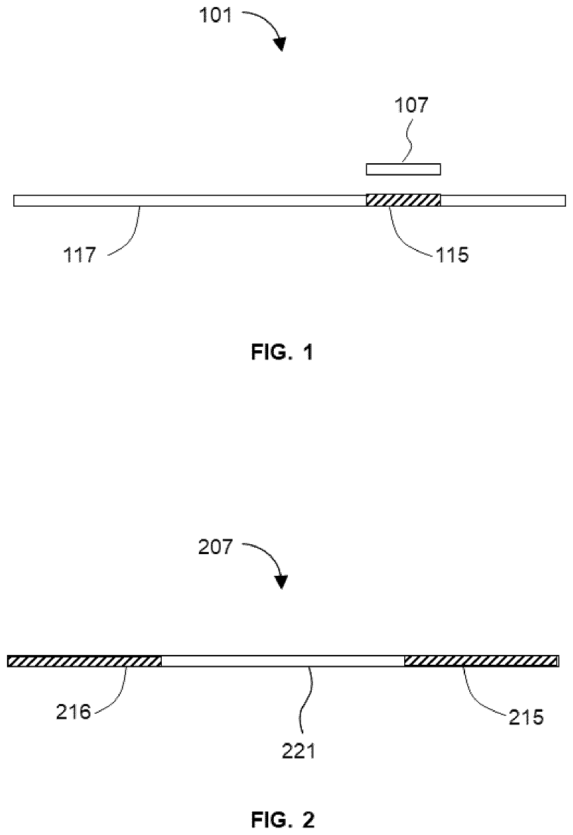

FIG. 1 shows a composition 101 for treating Dup15q Syndrome. The composition 101 includes an antisense oligonucleotide 107 that hybridizes to a target segment 115 in an mRNA 117 or a pre-mRNA. The RNA 117 encodes a ubiquitin ligase protein such as ubiquitin protein ligase E3A. The segment 115 of the RNA 117 that includes the target is at least about 75% complementary to one of SEQ ID NOS: 1-228. Hybridization of the ASO 107 to the segment 115 of the RNA 117 prevents translation of the mRNA into the UBE3A protein. Preferably, a sequence of bases in the oligonucleotide has at least 80% identity to one of SEQ ID NOS: 1-228, and more preferably at least about 90% identity. In certain embodiments, a sequence of bases in the oligonucleotide is at least about 90% identical to one of SEQ ID NOS: 1-228, wherein the oligonucleotide can hybridize to, and induce RNase H cleavage of UBE3A pre-mRNA or mRNA.

The oligonucleotide 107 hybridizes to the segment 115 in the mRNA 117 because the oligonucleotide 107 is substantially or entirely antisense to the target segment 115 of the mRNA 117. In that aspect, the composition includes an antisense oligonucleotide (ASO). Compositions 101 include ASOs that bind to target RNA with base pair complementarity and exert various effects, based on the ASO chemical structure and design. Various mechanisms, commonly employed in preclinical models of neurological disease and human clinical trial development, may be employed. Those mechanisms include RNA target degradation via recruitment of the RNaseH enzyme; alternative splicing modification to include or exclude exons, and miRNA inhibition to inhibit miRNA binding to its target.

Preferred embodiments of the disclosure include ASOs that hybridize to the UBE3A pre-mRNA or mRNA and recruit the RNaseH enzyme. The RNaseH enzyme cleaves the RNA, which downregulates expression of the UBE3A protein. Thus, oligonucleotide 107 of the disclosure addresses UBE3A CNVs as targets for Dup15q syndrome. The disclosure builds on the insights that data suggest that one of the most common genetic variants associated with autism spectrum disorder (ASD) are duplications of chromosome 15q11.2-q13.1 (Dup15q syndrome). The chromosome 15q11.2-q13.1 region includes the imprinted Prader-Willi/Angelman syndrome critical region (PWACR) as well as several genes critical for brain development and synaptic function, such as ubiquitin protein ligase E3A (UBE3A), small nuclear ribonucleoprotein polypeptide N (SNRPN), and three GABAA receptor genes (GABRB3, GABRA5, and GABRG3). Dup15q syndrome includes two primary types of duplications of 15q11.2-13.1: (1) an isodicentric chromosome 15 (idic(15)) that results in two additional maternally derived copies on a supernumerary chromosome that includes 15p and the proximal region of 15q11, most commonly leading to four copies of the region, or (2) an interstitial 15q duplication in which one extra copy of the 15q11.2-q13.1 region occurs on the same chromosome arm, typically resulting in three copies of the region, and has an overall milder phenotype. See Hogart, 2010, The comorbidity of autism with the genomic disorders of chromosome 15q11.2-13, Neurobiol Dis 38:181-91, incorporated by reference. Increased copy number for methylated maternal 15q duplications leads to changes in gene and protein expression and overexpression of UBE3A is linked to severity in Dup15q, where the increased number of maternal alleles is thought to be the primary driver of Dup15q pathology. See Scoles, 2011, Increased copy number for methylated maternal 15q duplications leads to changes in gene and protein expression in human cortical samples, Mol Autism 2:19 and Baker, 2020, Relationships between UBE3A and SNORD116 expression and features of autism in chromosome 15 imprinting disorders, Translational Psychiatry 10:362, both incorporated by reference. Here, compositions that include UBE3A ASOs are administered to a subject to treat Dup15q syndrome.

Thus, the disclosure provides a use of an antisense oligonucleotide (ASO) for the manufacture of a medicament for treating Dup15q syndrome in a patient. In the use, the ASO has at least about 75% identity with one of SEQ ID NOS: 1-228, and more preferably at least 90% identity, e.g., 95% or greater identity. Preferred embodiments use an ASO that is between about 15 and 25 bases in length, preferably between about 18 and 22 (inclusive). In general, reference to “an ASO” includes numerous copies of substantially identical molecules. Accordingly, “an ASO” may be more than hundreds of thousands or millions of copies of the defined ASO. In preferred embodiments, the ASO is 20 bases in length and has the sequence of one of SEQ ID NOS: 1-228 and is used in the manufacture of a medicament for the treatment of Dup15q syndrome. The ASO may be provided in any suitable format such as, for example, lyophilized in a tube or in solution in a tube, such as a microcentrifuge tube or a test tube. Preferred embodiments of the use target UBE3A. One or more (e.g., two, three, four, or five, or more) ASOs may be used in manufacture of the medicament. The one or more ASOs may hybridize to a target in a UBE3A mRNA. In certain embodiments of the use, a sequence of bases in the ASO is at least 90% identical to one of SEQ ID NOS: 1-228. In embodiments of the use, an ASO may have a gapmer structure with a central DNA segment flanked by RNA wings, e.g., a central region of 10-12 DNA bases with 4-5 modified RNA bases on both sides of the central region. Each modified RNA base may be 2′-MOE RNA, 2′-O-methyl RNA, or other suitable sugar. Preferably a backbone of the ASO has a plurality of phosphorothioate bonds, either exclusively or also including phosphodiester linkages, e.g., most or all of the sugar linkages may be phosphorothioate in the use embodiments. The ASO may initially be in a form suitable for mixing into a formulation suitable for introduction by injection. For example, the ASO (thousands or millions or more of copies of one ASO) may be lyophilized in a tube or in solution at a known quantity, molality, or concentration. The ASO may be dissolved or diluted into a pharmaceutically acceptable composition in which a carrier, such as a solvent or excipient, includes the ASO and may be loaded in an IV bag, syringe, or vial. The medicament may be made using more than one ASO, e.g., any combination of 2, 3, 4, or 5, or more.

Any ASO(s) described in the use embodiment may be included in a composition of the disclosure. Preferred embodiments of compositions of the disclosure include one or a plurality of therapeutic oligonucleotides each having a base sequence at least 80% identical to one of SEQ ID NOS: 1-228 wherein each of the therapeutic oligonucleotides has a gapmer structure that comprises a central DNA segment flanked by modified RNA wings, wherein the plurality of therapeutic oligonucleotides are provided in a solution or carrier formulated for injection.

FIG. 2 shows an oligonucleotide 207 with a gapmer structure. The oligonucleotide 207 includes two wings (first wing 215 and second wing 216) flanking a central region 221 of about 10-12 DNA bases. In preferred embodiments, the wings 215, 216 are all or predominantly RNA bases whereas the central region 221 is all or predominantly DNA bases. Preferably, the wings are all RNA bases (modified or unmodified) and the central region is all DNA bases. In some embodiments, each wing consists of 5 RNA bases, all or most of which are modified RNA bases, e.g., in which each modified RNA base is selected from the group consisting of 2′-O-methoxyethyl RNA and 2′-O-methyl RNA. A modified RNA base may include a substitution on a 2′ hydroxyl group of a ribose sugar. A 2′-O-Methoxyethyl (“2′-MOE”) modified sugar may be included in an RNA base. The oligonucleotide 207 preferably includes at least about 15 bases and may include between about 15 about 25 bases. In some embodiments, the oligonucleotide 207 has a backbone comprising a plurality of phosphorothioate bonds. One or any number of phosphorothioate bonds may be included in the backbone of a segment of DNA, such as the central region 221 of the oligonucleotide 207. The oligonucleotide 207 may include one or any number of the phosphorothioate bonds. For example, every backbone linkage within the oligonucleotide 207 may be phosphorothioate, or most, or about half may be phosporothioate. In addition, there may be other modifications to the phosphodiester backbone.

The composition 101 may be formulated for delivery. Accordingly, the oligonucleotide 107 may initially be in a form suitable for mixing into a formulation suitable for introduction into a syringe, bag, or injection pump. For example, the oligonucleotide 107 (thousands or millions or more of copies of one oligonucleotide 107) may be lyophilized in a tube or in solution at a known molality of concentration. The oligonucleotide 107 may be dissolved or diluted into a pharmaceutically acceptable composition in which a carrier, such as a solvent or excipient, includes the oligonucleotide 107 and may be loaded in an IV bag, syringe, or vial. As described, the composition 101 includes at least one oligonucleotide 107 with a sequence that is defined by comparison to one of SEQ ID NO: 1-228. Thus, compositions of the disclosure are defined and illustrated by the identified targets.

Specifically, the oligonucleotide 107 hybridizes to an mRNA encoding a UBE3A protein along a segment of the mRNA that is at least about 75% complementary to one of SEQ ID NOS: 1-228 to thereby prevent translation of the mRNA into the UBE3A protein. This is accomplished where the oligonucleotide has at least about 75% identity to one of SEQ ID NOS: 1-228, preferably at least about 90% or 95% or 100% identity. In certain embodiments, the oligonucleotide has the sequence of one of SEQ ID NOS: 1-228, although one of skill in the art will understand that oligonucleotides with 90 or preferably 95% identity to a complementary target will still tend to hybridize in a sequence-specific manner to the target. Forming a double stranded structure is energetically favorable enough through Watson-Crick base pairing and base stacking that the double stranded structure can tolerate approximately about 1 mismatched base pair every ten or so. Accordingly, under moderately stringent physiological conditions in a cell, 95% identity should be effective, especially where an oligonucleotide has a gapmer structure with at least a few modified RNA bases or phosphorothioate backbone linkages to protect the oligonucleotide from enzymatic degradation.

In fact, a feature and benefit of compositions of the disclosure is that the targets (of SEQ ID NOS: 1-228) have been substantially screened to rule out sequences for which the complement is present in molecules other than UBE3A transcripts. For example, the sequences have been screened against databases of RNA transcripts including long, non-coding RNA (lncRNA), and initial sequences that matched non-target sequences were excluded. Thus, ASOs with sequences of SEQ ID Nos. 1-228 when administered to a patient should have a minimized chance of hybridizing to non-target sequences. Accordingly, in preferred embodiments, the oligonucleotide 107 has a base sequence that has been screened and determined to not meet a threshold match for any off-target coding or long, non-coding RNA in humans. A composition or use that meets the criteria stated above should not bind to off-target material such as lncRNA or other off-target RNA transcripts in vivo, as the included sequences have been screened against a database of lncRNA and other RNA transcripts. Sequences of the disclosure have been screened for target specificity. Preferably, the oligonucleotide 107 has a base sequence with 0 mismatches to a homologous segment in a human or non-human primate genome and no more than about 5 mismatches in a homologous segment in a rodent genome.

When the composition is delivered to cells, the cells exhibit a dose-dependent knockdown of UBE3A.

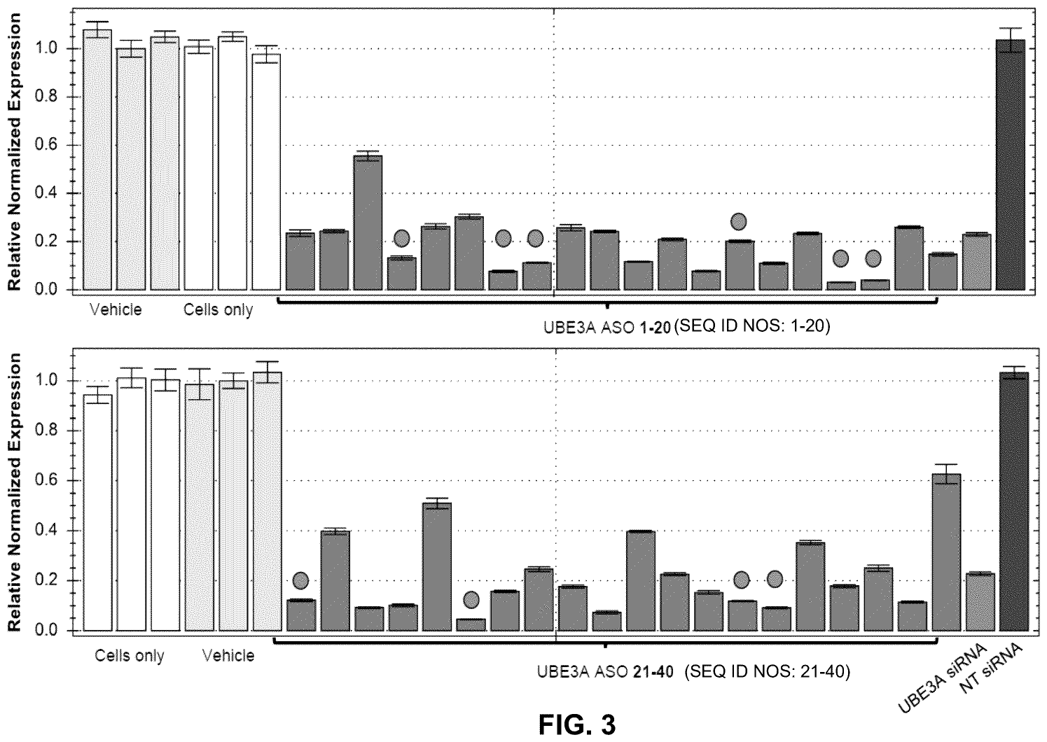

FIG. 3 shows results from screening 40 UBE3A exonic ASOs (with 1 control fibroblast line; results taken 48 hours posttreatment). The indicated results correspond to SEQ ID Nos. 1-40. In the figure, bars for ASOs that were tested in concentration response (CR) are marked by circles.

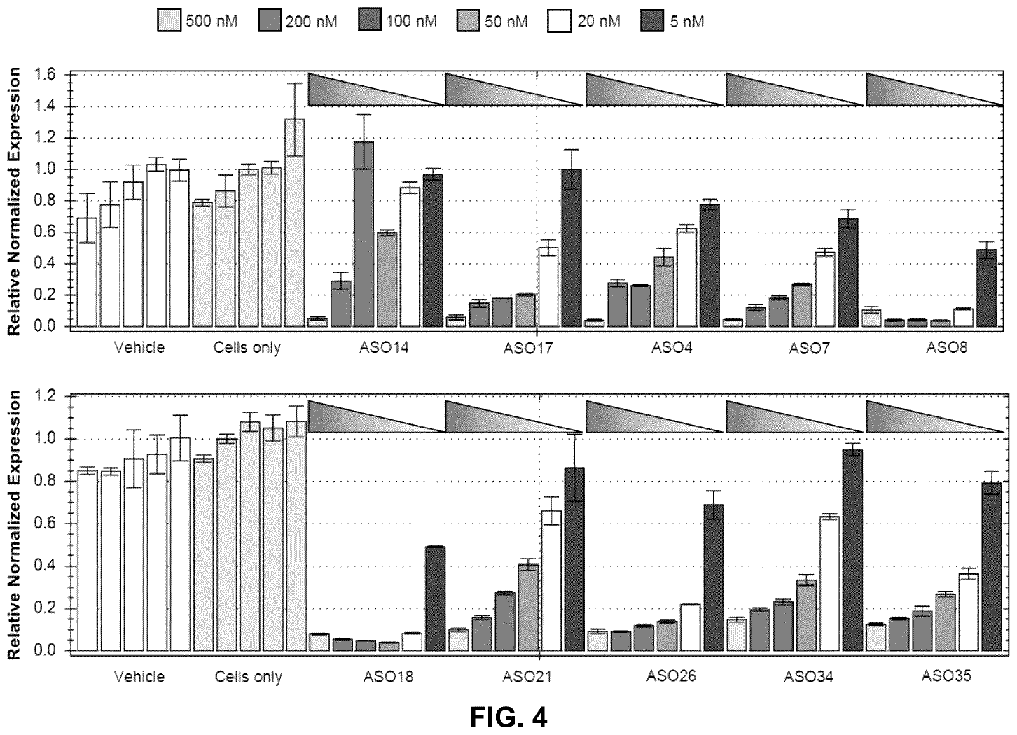

FIG. 4 gives results showing dose-response of ten ASO candidates of SEQ ID NOS: 14, 17, 4, 7, 8, 18, 21, 26, 34, and 35 (at 6 concentrations each) designed according to embodiments of the disclosure (about 20 bases, about 12 base DNA central region flanked by RNA wings with 2′-0 modified RNA and phosphorothioate linkages through ASO). A 11 ten ASOs decreased UBE3A expression, relative to controls in a dose-dependent manner (vehicle-only treated cells and untreated “cells only” conditions).

Because nucleic acid hybridization has some tolerance for mis-matches, it may be found that an oligonucleotide 107 with a base sequence that is at least a90% match to one of SEQ ID NOS: 1-228, with bases linked only by phosphorothioate linkages, and in which the oligonucleotide 107 has a central segment of DNA bases flanked by a 5′ wing and a 3′ wing (e.g., a 4-12-4 structure in which the 5′ wing and the 3′ wing each comprise four consecutive 2′ modified RNA bases flanking 12 DNA bases, or a 5-10-5 structure, or similar) exhibits dose-dependent knockdown according to the pattern shown in the chart. In some embodiments, the oligonucleotide 107 specifically has a base sequence matching one of SEQ ID NOS: 1-228 (more preferably one of SEQ ID NOS: 1-40 or more preferably SEQ ID NOS: 146, 155, 156, 158, 159, 161, 164, 169, 174, 175, 178, 179, 213, or 214), with bases linked by phosphorothioate linkages (optionally with some phosphodiester linkages), in which the oligonucleotide 107 has a central 12 DNA bases flanked by a 5′ wing and a 3′ wing, and in which the 5′ wing and the 3′ wing each include four consecutive 2′-MOE RNA bases.

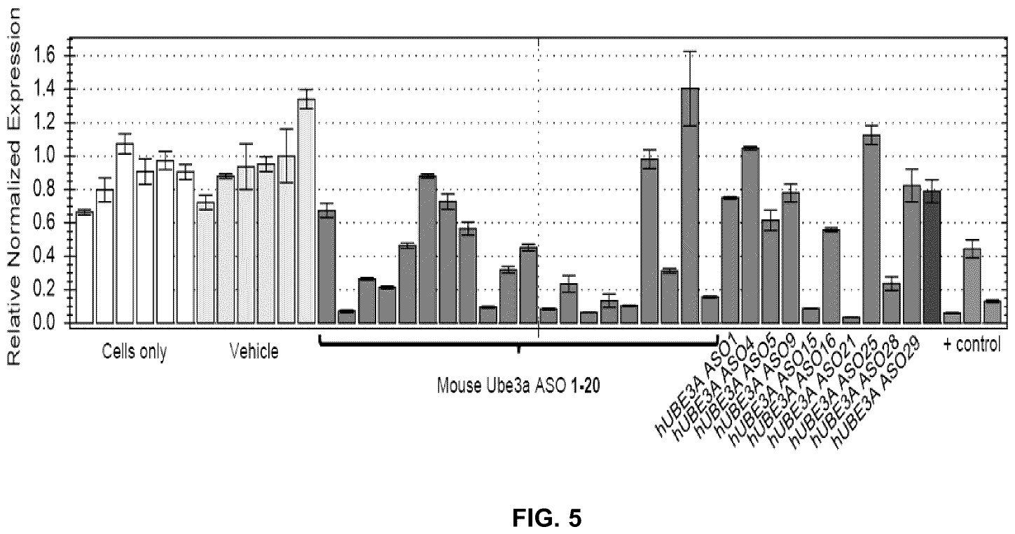

FIG. 5 shows results from screening mouse exonic Ube3a ASOs and human exonic ASOs with mouse homology in mouse fibroblasts. The screened human ASOs included those of SEQ ID NOS: 1, 4, 5, 9, 15, 16, 21, 25, 28, and 29. The results tend to show that it is possible to design ASOs against human targets for which there exist homologous targets in rodent models.

Because these compositions are effective at knocking down expression of UBE3A, the compositions of the disclosure may be used to treat Dup15q syndrome in patients. Methods of the disclosure include administering to a patient in need thereof any composition of the disclosure to thereby treat or alleviate Dup15q syndrome.

Compositions of the disclosure may be tested on in vitro samples of living neurons. For example, neurons in vitro may include optogenetic constructs that provide neural activation under optical stimulus (e.g., a modified algal channelrhodopsin that causes the neuron to fire in response to light) and optical reporters of neural activity (modified archaerhodopsins that emit light in proportion to neuronal membrane voltage and yield signals of neuronal activity). The in vitro neurons may be assayed in a fluorescence microscopy instrument and optionally treated with neural stimulant composition that causes neurons to fire in a predictable manner. Any suitable optogenetic constructs, optogenetic microscope, or neural stimulant compositions may be used. For example, suitable optogenetic constructs include those described in U.S. Pat. No. 9,594,075, incorporated by reference. Suitable optogenetic microscopes include those described in U.S. Pat. No. 10,288,863, incorporated by reference.

Methods and compositions of the disclosure may beneficially be used for delivery of therapeutic oligonucleotides 107 described herein to neurons in vivo in subjects with Dup15q syndrome. Any suitable delivery approach may be used including, for example, systemic delivery (e.g., by injection) or local delivery (e.g., by subcutaneous, intrathecal, or implantation of a slow-release device). Methods of the disclosure may involve delivering a composition of the disclosure once, several times over days or weeks, every few months, e.g., about 3 or 4 times per year.

An oligonucleotide of the disclosure, such as a gapmer, ASO, or therapeutic oligonucleotide 107 in a composition 101 may have a sequence defined with reference to one of the sequences set forth in Table 1. For example, an oligonucleotide of the disclosure may have a sequence that is at least about 75%, 80%, 90%, 95%, or perfectly identical to one of SEQ ID NOS: 1-228 as set forth in Table 1. Certain preferred embodiments against UBE3A include those in Table 1 labeled as SEQ ID NOS: 1-40.

Further, as described in the Examples presented below, the inventors screened ASOs of the invention. Based on the resulting data, ASOs corresponding to SEQ ID NOS: 146, 155, 156, 158, 159, 161, 164, 169, 174, 175, 178, 179, 213, and 214 were identified as lead candidate ASOs based on single dose and dose-response efficacy, sequence motif liabilities, and off-target alignment analyses. Those ASOs showed the greatest in vitro efficacy, lowest off-target alignments, and limited sequence motif concerns. Accordingly, in certain aspects, preferred ASOs against UBE3A according to the invention include ASOs having a sequence that is at least about 75%, 80%, 90%, 95%, or perfectly identical to a sequence corresponding to SEQ ID NOS: 146, 155, 156, 158, 159, 161, 164 169, 174, 175, 178, 179, 213, and 214.

TABLE 1
Sequences for ASOs

| | | Start position in negative |
|---|---|---|
| Sequence Identifier | Sequence | strand of chromosome 15 |
| SEQ ID NO: 1 | TCATTTCCACAGCCCTCAGT | 25375694 |
| SEQ ID NO: 2 | TCAGAGCAGGAGTTGTTGGG | 25375505 |
| SEQ ID NO: 3 | GATTTCAGTTCTTCCTTGGT | 25371643 |
| SEQ ID NO: 4 | TCCATAGCAGCAGCAGAACA | 25371571 |
| SEQ ID NO: 5 | GCTTCTGAGTCTTCTTCCAT | 25371556 |
| SEQ ID NO: 6 | GTGAGCTATCACCTATCCTT | 25371527 |
| SEQ ID NO: 7 | TTGTTGTCTCCCTGTGAGCT | 25371514 |
| SEQ ID NO: 8 | GCAATCTGGTGTAGACCCTT | 25371443 |
| SEQ ID NO: 9 | TCCCCTCCCACTACATTTGC | 25371022 |
| SEQ ID NO: 10 | TTTGTGTCCACTTCCCCTCC | 25371010 |
| SEQ ID NO: 11 | GGGATGGGCTCTTCATCATC | 25370977 |
| SEQ ID NO: 12 | AGGACCTTTCTTGTTTCTTC | 25370913 |
| SEQ ID NO: 13 | ACCAAGTTCAGTTTCCAGGG | 25370883 |
| SEQ ID NO: 14 | ACCTCATTCAGTGGTTCATT | 25370812 |
| SEQ ID NO: 15 | GGATTCAACTGCTGTCCTTG | 25370620 |
| SEQ ID NO: 16 | TCATCAACTCCTTGTTCTCC | 25360444 |
| SEQ ID NO: 17 | ATTTCCTCCACAACCAGCTG | 25360399 |
| SEQ ID NO: 18 | GCCAGACCCAGTACTATGCC | 25356793 |
| SEQ ID NO: 19 | CCACATTCCCTTCATACTCC | 25356007 |
| SEQ ID NO: 20 | GAGTCCCTGGTATAGCCACC | 25354364 |
| SEQ ID NO: 21 | AGTCTTTTCTGTTCATCTGT | 25340180 |
| SEQ ID NO: 22 | CAGGTGCTCTGTCTGTGCCC | 25340142 |
| SEQ ID NO: 23 | CCCACAGGTGCTCTGTCTGT | 25340138 |
| SEQ ID NO: 24 | CCTAGTCCTCCCACAGGTGC | 25340129 |
| SEQ ID NO: 25 | AACCTTTCTGTGTCTGGGCC | 25339254 |
| SEQ ID NO: 26 | CAGCCTTTTTGTACTGGGAC | 25339012 |
| SEQ ID NO: 27 | TTCCAGCCCACATGTCCCCA | 25338942 |
| SEQ ID NO: 28 | GAAATCTGCTGTTCCAGCCC | 25338931 |
| SEQ ID NO: 29 | AGGCTCAACCTCAAGCAGTA | 25338769 |
| SEQ ID NO: 30 | GGGAGAGTAGTTCTGTTGGT | 25338727 |
| SEQ ID NO: 31 | CATTCCAATTTCTCCCTTCC | 25338489 |
| SEQ ID NO: 32 | CCCTGTCCTTTCATATACTA | 25338344 |
| SEQ ID NO: 33 | GGCCAAATGCACTTTCCCCA | 25338284 |
| SEQ ID NO: 34 | GCACAGTAGCCATCTTTTTC | 25338041 |
| SEQ ID NO: 35 | TCATTCATTTCCAGGTCAGC | 25337996 |
| SEQ ID NO: 36 | AGGCACAAGCTCAGCACATT | 25337708 |
| SEQ ID NO: 37 | GCATTGTCTTCTTTTTCCAC | 25337455 |
| SEQ ID NO: 38 | CCCCATGTTACCTTATCACA | 25337426 |
| SEQ ID NO: 39 | GTCCCTTTCATCAAGGTAGC | 25337365 |
| SEQ ID NO: 40 | GCACAGTGGATGAGAAGCCT | 25337320 |
| SEQ ID NO: 41 | GCTGCTCGCTTCCTGTACCA | 25375752 |
| SEQ ID NO: 42 | CTTACTGGGTGAGAGTCTCC | 25356686 |
| SEQ ID NO: 43 | TTCTTACCCGGCTTCCACAT | 25354521 |
| SEQ ID NO: 44 | TTTCTTACCCGGCTTCCACA | 25354520 |
| SEQ ID NO: 45 | CTTTCTTACCCGGCTTCCAC | 25354519 |
| SEQ ID NO: 46 | TACCTTTCTGTGTCTGGGCC | 25340082 |
| SEQ ID NO: 47 | ACCTTCCTGTTTTCATTTGT | 25355890 |
| SEQ ID NO: 48 | ACTTACTGGGTGAGAGTCTC | 25356685 |
| SEQ ID NO: 49 | TACCTTCCTGTTTTCATTTG | 25355889 |
| SEQ ID NO: 50 | AACTTACTGGGTGAGAGTCT | 25356684 |
| SEQ ID NO: 51 | GCCCTCCCTTCCCATCAATC | 25438011 |
| SEQ ID NO: 52 | TCCCCACACCTCTGACTAGT | 25436704 |
| SEQ ID NO: 53 | GGGTGGTGGGCTGGGACCAA | 25435050 |
| SEQ ID NO: 54 | ACTGACCCCTAGTTCTGCCT | 25430565 |
| SEQ ID NO: 55 | CCTTGGCTCTCCCCTCCCTT | 25425998 |
| SEQ ID NO: 56 | GGACCCATGGCCTTTGAGCT | 25415877 |
| SEQ ID NO: 57 | TGACACCATACCTCCCCTCT | 25415825 |
| SEQ ID NO: 58 | CCCAGCACTACTGCCCACTA | 25415373 |
| SEQ ID NO: 59 | ACCCCAGCCATCCCAGCACT | 25415362 |
| SEQ ID NO: 60 | GAGTCTCTCTCTTTCCCAGT | 25414672 |
| SEQ ID NO: 61 | CCTCTGACCCTTGAGTCTCC | 25412413 |
| SEQ ID NO: 62 | CACCCTACCTGGGTCCCTCA | 25411743 |
| SEQ ID NO: 63 | CCTCTCTTCCAGTCCCCTCT | 25411061 |
| SEQ ID NO: 64 | GGTCAACTCTCAGGCCCACT | 25408962 |
| SEQ ID NO: 65 | GGTGCAGCTTCTCCATCCTG | 25408633 |
| SEQ ID NO: 66 | CCCTCCAGCATCAGATGTCA | 25407191 |
| SEQ ID NO: 67 | GACACACCTGGTCTCCACCA | 25407060 |
| SEQ ID NO: 68 | CTTCACCCATTCCCCTCAGT | 25403266 |
| SEQ ID NO: 69 | TGGGCTCCTGTGTCTGTCAG | 25393846 |
| SEQ ID NO: 70 | GCCCTCCAGTGACCCTGCCA | 25380443 |
| SEQ ID NO: 71 | GTCCAGGAGTCTTTCAGCTT | 25378642 |
| SEQ ID NO: 72 | CTGCATTCCACTGTGCCAGC | 25374354 |
| SEQ ID NO: 73 | GGGTCTTCCTAGTTTGTTCC | 25372328 |
| SEQ ID NO: 74 | GTTTCCTTATGCCAGTTCCC | 25362783 |
| SEQ ID NO: 75 | ATGAGCAGGGTCCAGCAGGA | 25342721 |
| SEQ ID NO: 76 | TTGCCACTTCCCTTCCCTGC | 25341989 |
| SEQ ID NO: 77 | GACTCTACACTGTCCAGCCA | 25432729 |
| SEQ ID NO: 78 | CTCCATTAGCTCCTCAGAGT | 25413636 |
| SEQ ID NO: 79 | TCCTCCTAACCTCTTCCAGA | 25397434 |
| SEQ ID NO: 80 | CCACATCTCAGCCATTCCTT | 25366556 |
| SEQ ID NO: 81 | GCTATCACCTATCCTTGA | 25371531 |
| SEQ ID NO: 82 | GTCTCCCTGTGAGCTATC | 25371519 |
| SEQ ID NO: 83 | TCTGGTGTAGACCCTTCT | 25371447 |
| SEQ ID NO: 84 | CCTCCCACTACATTTGCA | 25371025 |
| SEQ ID NO: 85 | ATTCAACTGCTGTCCTTG | 25370622 |
| SEQ ID NO: 86 | TGCAGGATTTTCCATAGC | 25360497 |
| SEQ ID NO: 87 | TAGCCAGACCCAGTACTA | 25356791 |
| SEQ ID NO: 88 | GTGAGAGTCTCCCAAGTC | 25356693 |
| SEQ ID NO: 89 | CACATTCCCTTCATACTC | 25356008 |
| SEQ ID NO: 90 | GGCTTCCACATATAAGCA | 25354529 |
| SEQ ID NO: 91 | ATCTGCTGTTCCAGCCCA | 25338934 |
| SEQ ID NO: 92 | GAGAGTAGTTCTGTTGGT | 25338729 |
| SEQ ID NO: 93 | ACATACTGTGGCATGAGT | 25338414 |
| SEQ ID NO: 94 | GCACTTTCCCCAGTAAAC | 25338292 |
| SEQ ID NO: 95 | GCAATAGGCTTGACTACC | 25338257 |
| SEQ ID NO: 96 | GGGAGACTTTGGATTGTC | 25338130 |
| SEQ ID NO: 97 | CCAGGTCAGCTTACTGTA | 25338006 |
| SEQ ID NO: 98 | GCTCAGCACATTAGCTAT | 25337716 |
| SEQ ID NO: 99 | CCCCATGTTACCTTATCA | 25337426 |
| SEQ ID NO: 100 | GGTCCCTTTCATCAAGGT | 25337364 |
| SEQ ID NO: 101 | GGAGGGATGAGGATCACAGA | |
| SEQ ID NO: 102 | GCTTGCTCCTTTCTTGGAGG | |
| SEQ ID NO: 103 | TATCTCAGAGCAGGAGTTGT | |
| SEQ ID NO: 104 | GCTCTGTACCAATGCCTCAG | |
| SEQ ID NO: 105 | CAGAACATGCAGCTTTTTCC | |
| SEQ ID NO: 106 | GCCATTTCCAGATATTCAGG | |
| SEQ ID NO: 107 | TCAGTTTTCCTTGGGCTGCA | |
| SEQ ID NO: 108 | GTTGCTGAAATGTCTCCATC | |
| SEQ ID NO: 109 | CCCTCCCACTACATTTGCAT | |
| SEQ ID NO: 110 | CTAGAACCTCATTCAGTGGT | |
| SEQ ID NO: 111 | GATTCAACTGCTGTCCTTGA | |
| SEQ ID NO: 112 | CCACATACAACTGCTTCTTC | |
| SEQ ID NO: 113 | CCAGACCCAGTACTATGCCA | |
| SEQ ID NO: 114 | TTCCCAGAACTCCCTAATCA | |
| SEQ ID NO: 115 | GGTAACCTTTCTGTGTCTGG | |
| SEQ ID NO: 116 | GGCCTTCAACAATCTCTCTT | |
| SEQ ID NO: 117 | GCCTTTTTGTACTGGGACAC | |
| SEQ ID NO: 118 | TCTGCTGTTCCAGCCCACAT | |
| SEQ ID NO: 119 | ATCTGCTGTTCCAGCCCACA | |
| SEQ ID NO: 120 | CTAAAGTTCTGAGGGCTGCA | |
| SEQ ID NO: 121 | CATACTGTGGCATGAGTTGT | |
| SEQ ID NO: 122 | GACTACCATTTCATTTGGCC | |
| SEQ ID NO: 123 | CATTTCCAGGTCAGCTTACT | |
| SEQ ID NO: 124 | CACCAAGGCACAAGCTCAGC | |
| SEQ ID NO: 125 | AAAGCTGCATTTTTCCTGCC | |
| SEQ ID NO: 126 | ACAGTGTTCTAAAGGCTGGC | |
| SEQ ID NO: 127 | CAGACACATCATCAGGGCCT | |
| SEQ ID NO: 128 | ACAGACACATCATCAGGGCC | |
| SEQ ID NO: 129 | CACAGACACATCATCAGGGC | |
| SEQ ID NO: 130 | GACTCAGGGATGGGCTCTTC | |
| SEQ ID NO: 131 | GGACTCAGGGATGGGCTCTT | |
| SEQ ID NO: 132 | TGGACTCAGGGATGGGCTCT | |
| SEQ ID NO: 133 | TCCCTTCCTTCCATCTTTCT | |
| SEQ ID NO: 134 | CTCCCTTCCTTCCATCTTTC | |
| SEQ ID NO: 135 | ACATACTGTGGCATGAGTTG | |
| SEQ ID NO: 136 | CAATCAGAGTAAACTGACCC | |
| SEQ ID NO: 137 | GACAGGAAGCACAAAACTCA | |
| SEQ ID NO: 138 | GGACAAGTGCATCATCTATG | |
| SEQ ID NO: 139 | TAAATAGCCAGACCCAGTAC | |
| SEQ ID NO: 140 | GGATTCAACTGCTGTCCTTG | |
| SEQ ID NO: 141 | GGATTCAACTGCTGTCCTTG | |
| SEQ ID NO: 142 | GGATTCAACTGCTGTCCTTG | |
| SEQ ID NO: 143 | AACCTTTCTGTGTCTGGGCC | |
| SEQ ID NO: 144 | AACCTTTCTGTGTCTGGGCC | |
| SEQ ID NO: 145 | AACCTTTCTGTGTCTGGGCC | |
| SEQ ID NO: 146 | GCTTGCTCCTTTCTTGGAGG | |
| SEQ ID NO: 147 | GCTTGCTCCTTTCTTGGAGG | |
| SEQ ID NO: 148 | GCTTGCTCCTTTCTTGGAGG | |
| SEQ ID NO: 149 | GGTAACCTTTCTGTGTCTGG | |
| SEQ ID NO: 150 | GGTAACCTTTCTGTGTCTGG | |
| SEQ ID NO: 151 | GGTAACCTTTCTGTGTCTGG | |
| SEQ ID NO: 152 | GGCCTTCAACAATCTCTCTT | |
| SEQ ID NO: 153 | GGCCTTCAACAATCTCTCTT | |
| SEQ ID NO: 154 | GGCCTTCAACAATCTCTCTT | |
| SEQ ID NO: 155 | GCAATCTGGTGTAGACCCTT | |
| SEQ ID NO: 156 | GCAATCTGGTGTAGACCCTT | |
| SEQ ID NO: 157 | GCAATCTGGTGTAGACCCTT | |
| SEQ ID NO: 158 | GGGATGGGCTCTTCATCATC | |
| SEQ ID NO: 159 | GGGATGGGCTCTTCATCATC | |
| SEQ ID NO: 160 | GGGATGGGCTCTTCATCATC | |
| SEQ ID NO: 161 | ACCAAGTTCAGTTTCCAGGG | |
| SEQ ID NO: 162 | ACCAAGTTCAGTTTCCAGGG | |
| SEQ ID NO: 163 | ACCAAGTTCAGTTTCCAGGG | |
| SEQ ID NO: 164 | GGATTCAACTGCTGTCCTTG | |
| SEQ ID NO: 165 | GGATTCAACTGCTGTCCTTG | |
| SEQ ID NO: 166 | ATTTCCTCCACAACCAGCTG | |
| SEQ ID NO: 167 | ATTTCCTCCACAACCAGCTG | |
| SEQ ID NO: 168 | ATTTCCTCCACAACCAGCTG | |
| SEQ ID NO: 169 | CAGCCTTTTTGTACTGGGAC | |
| SEQ ID NO: 170 | CAGCCTTTTTGTACTGGGAC | |
| SEQ ID NO: 171 | CAGCCTTTTTGTACTGGGAC | |
| SEQ ID NO: 172 | GCTTGCTCCTTTCTTGGAGG | |
| SEQ ID NO: 173 | GCTTGCTCCTTTCTTGGAGG | |
| SEQ ID NO: 174 | GCCATTTCCAGATATTCAGG | |
| SEQ ID NO: 175 | GCCATTTCCAGATATTCAGG | |
| SEQ ID NO: 176 | GCCATTTCCAGATATTCAGG | |
| SEQ ID NO: 177 | GGCCTTCAACAATCTCTCTT | |
| SEQ ID NO: 178 | GCCTTTTTGTACTGGGACAC | |
| SEQ ID NO: 179 | GCCTTTTTGTACTGGGACAC | |
| SEQ ID NO: 180 | GCCTTTTTGTACTGGGACAC | |
| SEQ ID NO: 181 | GACTACCATTTCATTTGGCC | |
| SEQ ID NO: 182 | GACTACCATTTCATTTGGCC | |
| SEQ ID NO: 183 | GACTACCATTTCATTTGGCC | |
| SEQ ID NO: 184 | TCATTTCCACAGCCCTCAGT | |
| SEQ ID NO: 185 | CCTTTCTTGGAGGGATGAGG | |
| SEQ ID NO: 186 | CTGAGCTTGCTCCTTTCTTG | |
| SEQ ID NO: 187 | GCAGCTTTTTCCTTTTCATC | |
| SEQ ID NO: 188 | CAGCAGCAGAACATGCAGCT | |
| SEQ ID NO: 189 | TCTTCTTCCATAGCAGCAGC | |
| SEQ ID NO: 190 | GATGCTTCTGAGTCTTCTTC | |
| SEQ ID NO: 191 | TCCCCTCCCACTACATTTGC | |
| SEQ ID NO: 192 | TCTGCAGGATTTTCCATAGC | |
| SEQ ID NO: 193 | ACTGCTTCTTCAAGTCTGCA | |
| SEQ ID NO: 194 | AGTCTTTTCTGTTCATCTGT | |
| SEQ ID NO: 195 | ACAGGTGCTCTGTCTGTGCC | |
| SEQ ID NO: 196 | CTGTGTCTGGGCCATTTTTG | |
| SEQ ID NO: 197 | ACCTTTCTGTGTCTGGGCCA | |
| SEQ ID NO: 198 | GTAGGTAACCTTTCTGTGTC | |
| SEQ ID NO: 199 | ACAGCCTTTTTGTACTGGGA | |
| SEQ ID NO: 200 | TGAAATCTGCTGTTCCAGCC | |
| SEQ ID NO: 201 | AGGCTCAACCTCAAGCAGTA | |
| SEQ ID NO: 202 | TCCCTGTCCTTTCATATACT | |
| SEQ ID NO: 203 | GCACTTTCCCCAGTAAACTT | |
| SEQ ID NO: 204 | CCTTTCTTGGAGGGATGAGG | |
| SEQ ID NO: 205 | CCTTTCTTGGAGGGATGAGG | |
| SEQ ID NO: 206 | CCTTTCTTGGAGGGATGAGG | |
| SEQ ID NO: 207 | ACAGGTGCTCTGTCTGTGCC | |
| SEQ ID NO: 208 | ACAGGTGCTCTGTCTGTGCC | |
| SEQ ID NO: 209 | ACAGGTGCTCTGTCTGTGCC | |
| SEQ ID NO: 210 | ACCTTTCTGTGTCTGGGCCA | |
| SEQ ID NO: 211 | ACCTTTCTGTGTCTGGGCCA | |
| SEQ ID NO: 212 | ACCTTTCTGTGTCTGGGCCA | |
| SEQ ID NO: 213 | ACAGCCTTTTTGTACTGGGA | |
| SEQ ID NO: 214 | ACAGCCTTTTTGTACTGGGA | |
| SEQ ID NO: 215 | ACAGCCTTTTTGTACTGGGA | |
| SEQ ID NO: 216 | GCACTTTCCCCAGTAAACTT | |
| SEQ ID NO: 217 | GCACTTTCCCCAGTAAACTT | |
| SEQ ID NO: 218 | GCACTTTCCCCAGTAAACTT | |
| SEQ ID NO: 219 | ACAGCCTTTTTGTACTGGGA | |
| SEQ ID NO: 220 | AAGGCTCAACCTCAAGCAGT | |
| SEQ ID NO: 221 | GGCTCAACCTCAAGCAGTAA | |
| SEQ ID NO: 222 | GCTCAACCTCAAGCAGTAAT | |
| SEQ ID NO: 223 | AATAGCCAGACCCAGTACTA | |
| SEQ ID NO: 224 | ATAGCCAGACCCAGTACTAT | |
| SEQ ID NO: 225 | CAGACCCAGTACTATGCCAA | |
| SEQ ID NO: 226 | CCAGTACTATGCCAATCAGA | |
| SEQ ID NO: 227 | GTGCAGGCTTCATTTCCACA | |
| SEQ ID NO: 228 | TTCCACAGCCCTCAGTTAAC | |

Preferred combination embodiments of the disclosure include a composition for treating Dup15q syndrome. The composition includes: a first oligonucleotide that hybridizes to an mRNA encoding the UBE3A protein along a segment of the mRNA that is at least about 90% complementary to one of SEQ ID NO: 1-40; and optionally a second oligonucleotide that hybridizes to an mRNA encoding a UBE3A protein along a segment of the mRNA that is at least about 90% complementary to a different one of SEQ ID NO: 1-40. In the preferred combination embodiments, each of the therapeutic oligonucleotides may have a gapmer structure that includes a central DNA segment flanked by modified RNA wings.

More preferred combination embodiments of the disclosure include a composition for treating Dup15q syndrome that includes an mRNA encoding a UBE3A protein along a segment of the mRNA that is at least about 90% complementary to one of SEQ ID NOS: 146, 155, 156, 158, 159, 161, 164, 169, 174, 175, 178, 179, 213, and 214; and optionally a second oligonucleotide that hybridizes to an mRNA encoding a UBE3A protein along a segment of the mRNA that is at least about 90% complementary to one of SEQ ID NOS: 146, 155, 156, 158, 159, 161, 164, 169, 174, 175, 178, 179, 213, and 214.

Either or both wings may include modified RNA bases, e.g., both wings may include 4 consecutive RNA bases with 2′-O-methoxyethyl ribose modifications. The entirety of each oligonucleotide may be connected via phosphodiester or phosphorothioate linkages or others as will be apparent to the skilled artisan. Most preferably, at least the terminal linkages will be non-standard (i.e., not phosphodiester, e.g., phosphorothioate) and more preferably most or all of the linkages within the RNA wings will be non-standard, e.g., phosphorothioate. Preferably the plurality of therapeutic oligonucleotides is provided lyophilized or in solution, for dilution or reconstitution in a clinic for delivery. That is, packaged in one or more tubes, lyophilized or in solution, are at least thousand to millions of copies of the first oligonucleotide and, optionally, at least thousand to millions of copies of the second oligonucleotide. This preferred combination embodiment of the composition may prove to have unexpected benefits as an antisense therapeutic for the treatment of Dup15q syndrome. Embodiments of the disclosure include oligonucleotides, including locked nucleic acid (LNA) antisense oligonucleotides targeting UBE3A which are capable of downregulating overexpression of UBE3A. The invention provides for an oligonucleotide of 10 to 30 nucleotides in length, which comprises a contiguous nucleotide sequence of 10 to 30 nucleotides in length with at least 90% complementarity, such as 100% complementarity, to a UBE3A target nucleic acid, and which is capable of inhibiting the overexpression of UBE3A in vivo. A n oligonucleotide 107 may be 100% identical to one of SEQ ID NOS: 1-228, or preferably one of SEQ ID NOS: 1-40 or one of SEQ ID NOS: 146, 155, 156, 158, 159, 161, 164, 169, 174, 175, 178, 179, 213, and 214. In certain aspects oligonucleotide 107 may be at least 90%, 95%, 98%, or 99% identical to one of SEQ ID NOS: 1-228, or preferably one of SEQ ID NOS: 1-40 or one of SEQ ID NOS: 146, 155, 156, 158, 159, 161, 164, 169, 174, 175, 178, 179, 213, and 214.

Embodiments include a pharmaceutically acceptable salt of the antisense oligonucleotide according to the invention, or the conjugate according to the invention.

The invention provides a pharmaceutical composition comprising the antisense oligonucleotide of the invention or the conjugate of the invention and a pharmaceutically acceptable diluent, solvent, carrier, salt and/or adjuvant.

The invention provides for the antisense oligonucleotide of the invention or the conjugate of the invention or the pharmaceutical salt or composition of the invention for use in medicine.

The invention provides for the antisense oligonucleotide of the invention or the conjugate of the invention or the pharmaceutical salt or composition of the invention for use in the treatment or prevention or alleviation of Dup15q syndrome. The invention provides for the use of the antisense oligonucleotide of the invention or the conjugate of the invention or the pharmaceutical salt or composition of the invention, for the preparation of a medicament for the treatment, prevention or alleviation of Dub15q syndrome.

Oligonucleotides are commonly made in the laboratory by solid-phase chemical synthesis followed by purification and isolation. When referring to a sequence of the oligonucleotide, reference is made to the sequence or order of nucleobase moieties, or modifications thereof, of the covalently linked nucleotides or nucleosides. The oligonucleotide of the invention may be man-made, i.e., chemically synthesized, and is typically purified or isolated. The oligonucleotide of the invention may comprise one or more modified nucleosides or nucleotides, such as 2′ sugar modified nucleosides.

The modified nucleotides may be independently selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxyl-modified nucleotide, a 2′-methoxyethyl modified nucleotide, a 2′-O-alkyl-modified nucleotide, a morpholino nucleotide, a phosphoramidate, a non-natural base comprising nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2′-O—(N-methylacetamide) modified nucleotide, and combinations thereof.

The nitrogenous bases of the ASO may be naturally occurring nucleobases such as adenine, guanine, cytosine, thymidine, uracil, xanthine and hypoxanthine, as well as non-naturally occurring variants, such as substituted purine or substituted pyrimidine, such as nucleobases selected from isocytosine, pseudoisocytosine, 5-methyl cytosine, 5-thiazolo-cytosine, 5-propynyl-cytosine, 5-propynyl-uracil, 5-bromouracil 5-thiazolo-uracil, 2-thio-uracil, 2′-thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine L N A nucleosides may be used.

A n oligonucleotide 107 of the disclosure is capable of down-regulating (inhibiting) the expression of UBE3A. In some embodiments the antisense oligonucleotide of the invention is capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target.

An antisense oligonucleotide (ASO) of the disclosure may decrease the level of the target nucleic acid (e.g., via RNaseH cleavage) or may decrease the functionality (or alter the functionality) of the target nucleic acid, e.g., via modulation of splicing of a pre-mRNA.

An oligonucleotide 107 of the disclosure may comprise one or more nucleosides which have a modified sugar moiety, i.e., a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance. Such modifications include those where the ribose ring structure is modified, e.g., by replacement with a hexose ring (HNA), or a bicyclic ring, which typically have a bridge between the C2 and C4 carbons on the ribose ring (LNA), or an unlinked ribose ring which typically lacks a bond between the C2 and C3 carbons (e.g., UNA). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

The oligonucleotide may include one or more Locked Nucleic Acid (LNA) bases. An LNA may include a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the L N A is incorporated into an oligonucleotide for a complementary RNA or DNA molecule. This can be routinely determined by measuring the melting temperature of the oligonucleotide/complement duplex. Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, and WO 2008/150729, all incorporated by reference.

Pharmaceutically acceptable salts of oligonucleotides of the disclosure include those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, a sulfonic acid, or salicylic acid. In addition, those salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins.

An oligonucleotide 107 may mediate or promote nuclease mediated degradation of UBE3A pre-mRNA or mRNA transcripts. Nuclease mediated degradation refers to an oligonucleotide capable of mediating degradation of a complementary nucleotide sequence when forming a duplex with such a sequence. In some embodiments, the oligonucleotide may function via nuclease mediated degradation of the target nucleic acid, where the oligonucleotides of the invention are capable of recruiting a nuclease, particularly an endonuclease, preferably endoribonuclease (RNase), such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers. The RNase H activity of an antisense oligonucleotide 107 refers to its ability to recruit RNase H when in a duplex with a complementary RNA molecule.

The antisense oligonucleotide 107 of the invention, or contiguous nucleotide sequence thereof, may be a gapmer, also termed gapmer oligonucleotide or gapmer designs. The antisense gapmers are commonly used to inhibit a target nucleic acid via RNaseH mediated degradation. A gapmer oligonucleotide comprises at least three distinct structural regions a 5′-flank, a gap and a 3′-flank, F-G-F′ in the ′5->3′ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e., are affinity enhancing sugar modified nucleosides). In some embodiments, the one or more sugar modified nucleosides in region F and F′ are 2′ sugar modified nucleosides, such as high affinity 2′ sugar modifications, such as independently selected from L N A and 2′-MOE.

A mixed wing gapmer is an L N A gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, 2′-MOE units, arabino nucleic acid (ANA) units, 2′-fluoro-A NA units, or combinations thereof. In some embodiments wherein at least one of region F and F′, or both region F and F′ comprise at least one LNA nucleoside, the remaining nucleosides of region F and F′ are independently selected from the group consisting of 2′-MOE and LNA. In some embodiments wherein at least one of region F and F′, or both region F and F′ comprise at least two LNA nucleosides, the remaining nucleosides of region F and F′ are independently selected from the group consisting of 2′-MOE and L N A. In some mixed wing embodiments, one or both of region F and F′ may further comprise one or more DNA nucleosides. Gapmer designs are discussed in WO 2008/049085 and WO 2012/109395, both incorporated by reference.

Table 2 shows examples of antisense oligonucleotides of the invention that incorporate modified bases and other modifications as described herein. As explained, numerous non-standard nucleic acid monomers are commercially available from custom oligonucleotide vendors and are easily incorporated into the antisense oligonucleotides of the invention. These monomer units are described using well-known oligonucleotide synthesis nomenclature to indicate the non-standard monomer units, for example as set forth by Integrated DNA Technologies (Iowa, US). For example, in the sequences provided in Table 2, the non-standard monomer units are enclosed in forward slashes “/” and an asterisk “*” between units indicates a PS linkage, while a lack of an asterisk indicates a PO linkage. Table 2 also provides the SEQ ID NO. of the ASO.

TABLE 2
Exemplary ASOs of the invention with modified nucleotides and linkages.

| SEQ ID | Sequence Showing Modifications |
|---|---|
| SEQ ID NO: 1 | /52M O E rT/*/12M O E rC/*/12MOErA/*/12MO E rT/*T*T*/iM e-dC/*/iM e- |
| | dC/*A*/iM e-dC/*A*G*/iM e-dC/*/iM e-dC/*/iM e- |
| | dC/*T*/12M OErC/*/12MOErA/*/12MOErG/*/32MOErT/ |
| SEQ ID NO: 2 | /52M O E rT/*/12M O ErC/*/12MOErA/*/12MOErG/*A*G*/IM e- |
| | dC/*A*G*G*A*G*T*T*G*T*/12MOErT/*/12MOErG/*/12MOErG/*/32 |
| | MOErG/ |
| SEQ ID NO: 3 | /52M O ErG/*/12M OErA/*/12MOErT/*/12MOErT/*T*/iM e- |
| | dC/*A*G*T*T*/IM e-dC/*T*T*/iM e-dC/*/iM e- |
| | dC/*T*/12MOErT/*/12MOErG/*/12MOErG/*/32MOErT/ |
| SEQ ID NO: 4 | /52M O E rT/*/12M O E rC/*/12M O ErC/*/12MOErA/*T*A*G*/IM e- |
| | dC/*A*G*/iM e-dC/*A*G*/iM e- |
| | dC/*A*G*/12MOErA/*/12MOErA/*/12MOErC/*/32M OErA/ |
| SEQ ID NO: 5 | /52M O ErG/*/12M OErC/*/12MOErT/*/12MOErT/*/iM e- |
| | dC/*T*G*A*G*T*/iM e-dC/*T*T*/iM e- |
| | dC/*T*T*/12MOErC/*/12MOErC/*/12MOErA/*/32MOErT/ |
| SEQ ID NO: 6 | /52M O ErG/*/i2M OErT/*/i2M OErG/*/12MOErA/*G*/iM e- |
| | dC/*T*A*T*/iM e-dC/*A*/iM e-dC/*/iM e- |
| | dC/*T*A*T*/12MOErC/*/12MOErC/*/12MOErT/*/32MOErT/ |
| SEQ ID NO: 7 | /52M O E rT/*/i2M OE rT/*/i2M OErG/*/12MOET/*T*G*T*/iMe- |
| | dC/*T*/iM e-dC/*/iM e-dC/*/iM e- |
| | dC/*T*G*T*G*/12MOErA/*/12MOErG/*/12MOErC/*/32MOET/ |
| SEQ ID NO: 8 | /52M O ErG/*/i2M O ErC/*/12MOErA/*/12MOErA/*T*/IM e- |
| | dC/*T*G*G*T*G*T*A*G*A*/IMe- |
| | dC/*/12MOErC/*/12MOErC/*/12MOErT/*/32MOErT/ |
| SEQ ID NO: 9 | /52M O E rT/*/12M O E rC/*/12MOErC/*/12MOErC/*/iM e-dC/*T*/iM e- |
| | dC/*/iM e-dC/*/iM e-dC/*A*/iM e-dC/*T*A*/iM e- |
| | dC/*A*T*/12M OErT/*/12MOErT/*/12MOErG/*/32M OErC/ |
| SEQ ID NO: 10 | /52M OErT/*/i2M O ErT/*/12M OErT/*/12M OErG/*T*G*T*/iM e- |
| | dC/*/iM e-dC/*A*/iM e-dC/*T*T*/iM e-dC/*/iM e-dC/*/iM e- |
| | dC/*/12MOErC/*/12MOErT/*/12MOErC/*/32MOErC/ |
| SEQ ID NO: 11 | /52M O ErG/*/i2M O ErG/*/12M OErG/*/12MOErA/*T*G*G*G*/iM e- |
| | dC/*T*/iM e-dC/*T*T*/iM e- |
| | dC/*A*T*/12M OErC/*/12MOErA/*/12MOErT/*/32MOErC/ |
| SEQ ID NO: 12 | /52M O ErA/*/12M O ErG/*/12M O ErG/*/12MOErA/*/iM e-dC/*/iM e- |
| | dC/*T*T*T*/IM e- |
| | dC/*T*T*G*T*T*T*/12MOErC/*/12MOErT/*/12MOErT/*/32MOErC/ |
| SEQ ID NO: 13 | /52M O ErA/*/12M O ErC/*/12M OE rC/*/12MOErA/*A *G*T*T*/iM e- |
| | dC/*A*G*T*T*T*/iM e-dC/*/iM e- |
| | dC/*/12MOErA/*/12M OErG/*/12MOErG/*/32MOErG/ |
| SEQ ID NO: 14 | /52M OErA/*/12M O ErC/*/12MOErC/*/12MOErT/*/iM e- |
| | dC/*A*T*T*/IM e- |
| | dC/*A*G*T*G*G*T*T*/12MOErC/*/i2MOErA/*/12MOErT/*/32MOErT/ |
| SEQ ID NO: 15 | /52M O ErG/*/i2M O ErG/*/12MOErA/*/12MOErT/*T*/iM e- |
| | dC/*A*A*/iM e-dC/*T*G*/iM e-dC/*T*G*T*/iM e- |
| | dC/*/12MOErC/*/12MOErT/*/12MOErT/*/32MOErG/ |
| SEQ ID NO: 16 | /52M O E rT/*/12M O ErC/*/12MOErA/*/12MOErT/*/iM e-dC/*A *A */IM e- |
| | dC/*T*/iM e-dC/*/iM e- |
| | dC/*T*T*G*T*T*/12MOErC/*/12MOErT/*/12MOErC/*/32MOErC/ |
| SEQ ID NO: 17 | /52M O ErA/*/12M O ErT/*/i2M OErT/*/i2M O ErT/*/iM e-dC/*/iM e- |
| | dC/*T*/iM e-dC/*/iM e-dC/*A*/iM e-dC/*A*A*/iM e-dC/*/iM e- |
| | dC/*A*/12MOErG/*/12MOErC/*/12MOErT/*/32M OErG/ |
| SEQ ID NO: 18 | /52M O ErG/*/12M O ErC/*/12M OErC/*/12MOErA/*G*A*/iM e-dC/*/iM e- |
| | dC/*/iM e-dC/*A*G*T*A*/IM e- |
| | dC/*T*A*/12MOErT/*/12MOErG/*/12MOErC/*/32MOErC/ |
| SEQ ID NO: 19 | /52M O E rC/*/12M O ErC/*/12MOErA/*/12MOErC/*A*T*T*/iM e- |
| | dC/*/iM e-dC/*/iM e-dC/*T*T*/iM e- |
| | dC/*A*T*A*/12MOErC/*/12MOErT/*/12MOErC/*/32MOErC/ |
| SEQ ID NO: 20 | /52M O ErG/*/12M OErA/*/i2M O ErG/*/12MOErT/*/iM e-dC/*/iM e- |
| | dC/*/iM e-dC/*T*G*G*T*A*T*A*G*/iMe- |
| | dC/*/12M OErC/*/12MOErA/*/12MOErC/*/32MOErC/ |
| SEQ ID NO: 21 | /52M OErA/*/i2M O ErG/*/12MOET/*/12MOErC/*T*T*T*T*/iMe- |
| | dC/*T*G*T*T*/iMe- |
| | dC/*A*T*/12MOErC/*/12MOErT/*/12MOErG/*/32MOErT/ |
| SEQ ID NO: 22 | /52M O E rC/*/12M O ErA/*/12M OErG/*/12M O ErG/*T*G*/iM e- |
| | dC/*T*/iM e-dC/*T*G*T*/IM e- |
| | dC/*T*G*T*/12MOErG/*/12MOErC/*/12MOErC/*/32MOErC/ |
| SEQ ID NO: 23 | /52M O ErC/*/12MOErC/*/12MOErC/*/12MOErA/*/iM e- |
| | dC/*A*G*G*T*G*/iM e-dC/*T*/iM e- |
| | dC/*T*G*T*/12MOErC/*/12MOErT/*/12MOErG/*/32MOErT/ |
| SEQ ID NO: 24 | /52M O E rC/*/12M O ErC/*/12M OErT/*/12MOErA/*G*T*/iM e-dC/*/iM e- |
| | dC/*T*/iM e-dC/*/iM e-dC/*/iM e-dC/*A*/iM e- |
| | dC/*A*G*/12MOErG/*/12MOErT/*/12MOErG/*/32MOErC/ |
| SEQ ID NO: 25 | /52M O ErA/*/i2M OErA/*/12M O E rC/*/12M OErC/*T*T*T*/IM e- |
| | dC/*T*G*T*G*T*/iMe- |
| | dC/*T*G*/12MOErG/*/i2M OErG/*/12MOErC/*/32MOErC/ |
| SEQ ID NO: 26 | /52M O ErC/*/12MOErA/*/12M OErG/*/12M OErC/*/iM e- |
| | dC/*T*T*T*T*T*G*T*A*/IMe- |
| | dC/*T*G*/12MOErG/*/12M OErG/*/12MOErA/*/32MOErC/ |
| SEQ ID NO: 27 | /52M O E rT/*/12M O E rT/*/i2M O E rC/*/12MO ErC/*A*G*/iM e-dC/*/iM e- |
| | dC/*/iM e-dC/*A*/iM e-dC/*A*T*G*T*/iMe- |
| | dC/*/12M OErC/*/12MOErC/*/12MOErC/*/32MOErA/ |
| SEQ ID NO: 28 | /52M O ErG/*/12M OErA/*/12M OErA/*/12MOErA/*T*/iM e- |
| | dC/*T*G*/iM e-dC/*T*G*T*T*/iM e-dC/*/iM e- |
| | dC/*A*/12MOErG/*/12MOErC/*/12MOErC/*/32MOErC/ |
| SEQ ID NO: 29 | /52M O ErA/*/12M O ErG/*/12MOErG/*/12MOErC/*T*/iM e- |
| | dC/*A*A*/iM e-dC/*/iM e-dC/*T*/iM e-dC/*A*A*G*/iMe- |
| | dC/*/12MOErA/*/12M OErG/*/12MOErT/*/32MOErA/ |
| SEQ ID NO: 30 | /52M O ErG/*/12M OErG/*/12M OErG/*/12MOErA/*G*A *G*T*A *G*T*T |
| | */iM e-dC/*T*G*T*/12MOErT/*/12MOErG/*/12MOErG/*/32MOErT/ |
| SEQ ID NO: 31 | /52M O ErC/*/i2M OErA/*/12MOErT/*/12MOErT/*/iM e-dC/*/iM e- |
| | dC/*A*A*T*T*T*/iM e-dC/*T*/iM e-dC/*/iM e-dC/*/iM e- |
| | dC/*/12MOErT/*/12MOErT/*/12MOErC/*/32MOErC/ |
| SEQ ID NO: 32 | /52M O E rC/*/12M O ErC/*/12MO ErC/*/12MOErT/*G*T*/iM e-dC/*/iM e- |
| | dC/*T*T*T*/iM e- |
| | dC/*A*T*A*T*/12MOErA/*/12MOErC/*/i2MOErT/*/32M OErA/ |
| SEQ ID NO: 33 | /52M O ErG/*/12M O ErG/*/i2M OErC/*/12MOErC/*A*A*A*T*G*/iMe- |
| | dC/*A*/iM e-dC/*T*T*T*/iM e- |
| | dC/*/12M O ErC/*/12MOErC/*/12MOErC/*/32MOErA/ |
| SEQ ID NO: 34 | /52M O ErG/*/12M O ErC/*/12M OErA/*/12M O ErC/*A *G*T*A*G*/iM e- |
| | dC/*/iM e-dC/*A*T*/iM e- |
| | dC/*T*T*/12MOErT/*/12MOErT/*/12MOErT/*/32M OErC/ |
| SEQ ID NO: 35 | /52M O E rT/*/12M O ErC/*/12MOErA/*/12MOErT/*T*/iM e- |
| | dC/*A*T*T*T*/iM e-dC/*/iM e- |
| | dC/*A*G*G*T*/12MOErC/*/12MOErA/*/12MOErG/*/32MOErC/ |
| SEQ ID NO: 36 | /52M OErA/*/12M O ErG/*/12M O ErG/*/12M OErC/*A*/iM e- |
| | dC/*A*A*G*/iM e-dC/*T*/iM e-dC/*A*G*/iM e- |
| | dC/*A*/12M OErC/*/12MOErA/*/12MOErT/*/32MOErT/ |
| SEQ ID NO: 37 | /52M O ErG/*/12M O ErC/*/12MOErA/*/12MOErT/*T*G*T*/iM e- |
| | dC/*T*T*/iM e- |
| | dC/*T*T*T*T*T*/12MOErC/*/12MOErC/*/12MOErA/*/32MOErC/ |
| SEQ ID NO: 38 | /52M O E rC/*/12M O E rC/*/12MOErC/*/12MOErC/*A*T*G*T*T*A*/IM e- |
| | dC/*/iM e- |
| | dC/*T*T*A*T*/12MOErC/*/12MOErA/*/12MOErC/*/32MOErA/ |
| SEQ ID NO: 39 | /52M O ErG/*/i2M O ErT/*/12M OErC/*/12M OErC/*/iM e- |
| | dC/*T*T*T*/iM e-dC/*A*T*/iM e- |
| | dC/*A*A*G*G*/12MOErT/*/12MOErA/*/i2M OErG/*/32MOErC/ |
| SEQ ID NO: 40 | /52M OErG/*/12M O ErC/*/12MOErA/*/12M OErC/*A *G*T*G*G*A*T*G |
| | *A *G*A*A*/i2MOErG/*/12M OErC/*/i2MOErC/*/32MOErT/ |
| SEQ ID NO: 41 | /52M O ErG/*/12M O ErC/*/12M O ErT/*/12M OErG/*/iM e-dC/*T*/iM e- |
| | dC/*G*/iM e-dC/*T*T*/iM e-dC/*/iM e- |
| | dC/*T*G*T*/12MOErA/*/12MOErC/*/12MOErC/*/32MOErA/ |
| SEQ ID NO: 42 | /52M O E rC/*/12M OErT/*/12MO E rT/*/i2M OErA/*/iM e- |
| | dC/*T*G*G*G*T*G*A*G*A*G*T*/12MOErC/*/12MOErT/*/12MOErC/ |
| | */32M OErC/ |
| SEQ ID NO: 43 | /52M O E rT/*/12M O E rT/*/12M O ErC/*/12M OErT/*T*A */iM e-dC/*/iM e- |
| | dC/*/iM e-dC/*G*G*/iM e-dC/*T*T*/iM e-dC/*/iMe- |
| | dC/*/12MOErA/*/12M OErC/*/12MOErA/*/32M OErT/ |
| SEQ ID NO: 44 | /52M O E rT/*/12M O E rT/*/12MOErT/*/12MOE rC/*T*T*A*/iM e- |
| | dC/*/iM e-dC/*/iM e-dC/*G*G*/iM e-dC/*T*T*/iM e- |
| | dC/*/12MOErC/*/12MOErA/*/12MOErC/*/32MOErA/ |
| SEQ ID NO: 45 | /52M O E rC/*/12MO E rT/*/12MOE rT/*/2M OErT/*/iM e- |
| | dC/*T*T*A*/iM e-dC/*/iM e-dC/*G*G*/iM e- |
| | dC/*T*T/12MOErC/*/12MOErC/*/12MOErA/*/32MOErC/ |
| SEQ ID NO: 46 | /52M O E rT/*/i2M OErA/*/i2M O E rC/*/12MOErC/*T*T*T*/iM e- |
| | dC/*T*G*T*G*T*/IM e- |
| | dC/*T*G*/12MOErG/*/12MOErG/*/12MOErC/*/32M OErC/ |
| SEQ ID NO: 47 | /52M OErA/*/12M O ErC/*/i2M OErC/*/12MOE rT/*T*/iM e-dC/*/iM e- |
| | dC/*T*G*T*T*T*T*/IMe- |
| | dC/*A*T*/12MOErT/*/i2MOErT/*/i2MOErG/*/32MOErT/ |
| SEQ ID NO: 48 | /52M OErA/*/12M O ErC/*/12MOErT/*/12MOErT/*A*/IM e- |
| | dC/*T*G*G*G*T*G*A*G*A*G*/12MOErT/*/12MOErC/*/12MOErT/*/ |
| | 32M OErC/ |
| SEQ ID NO: 49 | /52M O E rT/*/12M O ErA/*/12M O E rC/*/12M O ErC/*T*T*/iM e-dC/*/iM e- |
| | dC/*T*G*T*T*T*/iMe- |
| | dC/*A*/12MOErT/*/12MOErT/*/12MOErT/*/32M OErG/ |
| SEQ ID NO: 50 | /52M O ErA/*/12M O ErA/*/12M O ErC/*/12MOET/*T*A*/IM e- |
| | dC/*T*G*G*G*T*G*A*G*A*/12MOErG/*/12MOErT/*/i2MOErC/*/32 |
| | MOErT/ |
| SEQ ID NO: 51 | /52M O ErG/*/12M O ErC/*/12M OErC/*/i2M OErC/*T*/iM e-dC/*/iM e- |
| | dC/*/iM e-dC/*T*T*/iM e-dC/*/iM e-dC/*/iM e-dC/*A*T*/iM e- |
| | dC/*/12MOErA/*/12MOErA/*/12MOErT/*/32M OErC/ |
| SEQ ID NO: 52 | /52M O E rT/*/12M O E rC/*/12M O ErC/*/12MOErC/*/iM e-dC/*A*/iM e- |
| | dC/*A*/iM e-dC/*/iM e-dC/*T*/iM e-dC/*T*G*A*/iM e- |
| | dC/*/12MOErT/*/12MOErA/*/12MOErG/*/32MOErT/ |
| SEQ ID NO: 53 | /52M O ErG/*/i2M O ErG/*/12MOErG/*/12MOET/*G*G*T*G*G*G*/iMe |
| | -dC/*T*G*G*G*A*/12MOErC/*/12MOErC/*/12MOErC/*/32MOErA/ |
| SEQ ID NO: 54 | /52M O ErA/*/12M O ErC/*/i2M OErT/*/12MOErG/*A*/iM e-dC/*/iM e- |
| | dC/*/iM e-dC/*/iM e-dC/*T*A*G*T*T*/IM e- |
| | dC/*T*/12MOErG/*/12MOErC/*/12MOErC/*/32MOErT/ |
| SEQ ID NO: 55 | /52M O E rC/*/i2M O E rC/*/12MOET/*/12MOErT/*G*G*/iM e- |
| | dC/*T*/IM e-dC/*T*/iM e-dC/*/iM e-dC/*/iM e-dC/*/iM e-dC/*T*/iM e- |
| | dC/*/12MOErC/*/i2MOErC/*/12MOErT/*/32MOErT/ |
| SEQ ID NO: 56 | /52M O ErG/*/12M O ErG/*/12MOErA/*/12M O E rC/*/iM e-dC/*/iM e- |
| | dC/*A*T*G*G*/iMe-dC/*/iM e- |
| | dC/*T*T*T*G*/12MOErA/*/12MOErG/*/12MOErC/*/32MOErT/ |
| SEQ ID NO: 57 | /52M O E rT/*/12M OErG/*/12MOErA/*/12M OErC/*A*/iM e-dC/*/iM e- |
| | dC/*A*T*A*/iM e-dC/*/iM e-dC/*T*/iM e-dC/*/iM e-dC/*/iM e- |
| | dC/*/12MOErC/*/12MOErT/*/i2M OErC/*/32MOErT/ |
| SEQ ID NO: 58 | /52M O E rC/*/12MO ErC/*/12M OErC/*/12MOErA/*G*/iM e-dC/*A*/iM e- |
| | dC/*T*A*/iM e-dC/*T*G*/iM e-dC/*/iM e-dC/*/iM e- |
| | dC/*/12MOErA/*/12M OErC/*/12MOErT/*/32M OErA/ |
| SEQ ID NO: 59 | /52M O ErA/*/i2M O ErC/*/12M O E rC/*/12M O ErC/*/iM e-dC/*A*G*/iM e- |
| | dC/*/iM e-dC/*A*T*/iM e-dC/*/iM e-dC/*/iM e- |
| | dC/*A*G*/12MOErC/*/12MOErA/*/12MOErC/*/32MOErT/ |
| SEQ ID NO: 60 | /52M O ErG/*/12M O ErA/*/12MOErG/*/12MOErT/*/iM e-dC/*T*/iM e- |
| | dC/*T*/iM e-dC/*T*/iM e-dC/*T*T*T*/iM e-dC/*/iM e- |
| | dC/*/12MOErC/*/12MOErA/*/12MOErG/*/32MOErT/ |
| SEQ ID NO: 61 | /52M O ErC/*/12M O ErC/*/12MOErT/*/12MOErC/*T*G*A*/IM e- |
| | dC/*/iM e-dC/*/iM e- |
| | dC/*T*T*G*A*G*T*/12MOErC/*/12MOErT/*/12MOErC/*/32MOErC/ |
| SEQ ID NO: 62 | /52M O E rC/*/12M OErA/*/12M OE rC/*/12MOE rC/*/iM e-dC/*T*A*/iM e- |
| | dC/*/iM e-dC/*T*G*G*G*T*/iM e-dC/*/iM e- |
| | dC/*/12MOErC/*/12MOErT/*/12M OErC/*/32MOErA/ |
| SEQ ID NO: 63 | /52M OE rC/*/12M O E rC/*/12MOErT/*/12M OErC/*T*/iM e- |
| | dC/*T*T*/iM e-dC/*/iM e-dC/*A*G*T*/iM e-dC/*/iM e-dC/*/iM e- |
| | dC/*/12MOErC/*/12MOErT/*/12M OErC/*/32MOErT/ |
| SEQ ID NO: 64 | /52M O ErG/*/12M O ErG/*/12MOE rT/*/12MOErC/*A*A*/IM e- |
| | dC/*T*/iM e-dC/*T*/iM e-dC/*A*G*G*/iM e-dC/*/iM e- |
| | dC/*/12MOErC/*/12MOErA/*/12MOErC/*/32MOErT/ |
| SEQ ID NO: 65 | /52M O ErG/*/12M O ErG/*/12MOE rT/*/12MOErG/*/iM e-dC/*A*G*/iM e- |
| | dC/*T*T*/iM e-dC/*T*/iM e-dC/*/iM e- |
| | dC/*A*T*/12M OErC/*/12MOErC/*/12MOErT/*/32MOErG/ |
| SEQ ID NO: 66 | /52M O E rC/*/12M O E rC/*/i2M OErC/*/12MOErT/*/iM e-dC/*/iM e- |
| | dC/*A*G*/iM e-dC/*A*T*/iM e- |
| | dC/*A*G*A*T*/12MOErG/*/12MOErT/*/i2MOErC/*/32M OErA/ |
| SEQ ID NO: 67 | /52M O ErG/*/12M OErA/*/i2M O ErC/*/12MOErA/*/iM e-dC/*A */iM e- |
| | dC/*/iM e-dC/*T*G*G*T*/IM e-dC/*T*/iM e-dC/*/iM e- |
| | dC/*/12MOErA/*/12M OErC/*/12MOErC/*/32MOErA/ |
| SEQ ID NO: 68 | /52M O E rC/*/i2M OErT/*/12MOErT/*/12M OErC/*A*/iM e-dC/*/iM e- |
| | dC/*/iM e-dC/*A*T*T*/iM e-dC/*/iM e-dC/*/iM e-dC/*/iM e- |
| | dC/T*/12M OErC/*/12MOErA/*/12MOErG/*/32MOErT/ |
| SEQ ID NO: 69 | /52M O E rT/*/i2M O ErG/*/i2M OErG/*/12M O ErG/*/iM e-dC/*T*/iM e- |
| | dC/*/iM e-dC/*T*G*T*G*T*/IM e- |
| | dC/*T*G*/12MOErT/*/12MOErC/*/12MOErA/*/32MOErG/ |
| SEQ ID NO: 70 | /52M O ErG/*/12M O ErC/*/12MOE rC/*/12MOErC/*T*/iM e-dC/*/iM e- |
| | dC/*A*G*T*G*A*/iM e-dC/*/iM e-dC/*/iM e- |
| | dC/T*/12M OErG/*/12MOErC/*/12MOErC/*/32MOErA/ |
| SEQ ID NO: 71 | /52M O ErG/*/i2M O ErT/*/i2M OErC/*/12MOErC/*A*G*G*A*G*T*/iMe |
| | -dC/*T*T*T*/IM e- |
| | dC/*A*/12MOErG/*/12MOErC/*/12MOErT/*/32MOErT/ |
| SEQ ID NO: 72 | /52M O E rC/*/12M OErT/*/i2M OErG/*/12MOErC/*A*T*T*/iMe- |
| | dC/*/iM e-dC/*A*/iM e-dC/*T*G*T*G*/iM e- |
| | dC/*/12MOErC/*/12MOErA/*/12MOErG/*/32MOErC/ |
| SEQ ID NO: 73 | /52M O ErG/*/12M O ErG/*/12M OErG/*/12MOErT/*/iM e-dC/*T*T*/iM e- |
| | dC/*/iM e- |
| | dC/*T*A*G*T*T*T*G*/12MOErT/*/12MOErT/*/i2MOErC/*/32MOErC/ |
| SEQ ID NO: 74 | /52M O ErG/*/12M OErT/*/12M OE rT/*/12MOErT/*/iM e-dC/*/iM e- |
| | dC/*T*T*A*T*G*/iM e-dC/*/iM e- |
| | dC/*A*G*T*/12MOErT/*/12MOErC/*/12MOErC/*/32MOErC/ |
| SEQ ID NO: 75 | /52M OErA/*/12M O ErT/*/12M OErG/*/12MOErA/*G*/iM e- |
| | dC/*A*G*G*G*T*/iMe-dC/*/iM e-dC/*A*G*/iM e- |
| | dC/*/12MOErA/*/12M OErG/*/12M OErG/*/32MOErA/ |
| SEQ ID NO: 76 | /52M O E rT/*/12M O E rT/*/12M O ErG/*/12M OErC/*/iM e-dC/*A */iM e- |
| | dC/*T*T*/iM e-dC/*/iM e-dC/*/iM e-dC/*T*T*/iM e-dC/*/iM e- |
| | dC/*/12MOErC/*/12MOErT/*/12MOErG/*/32MOErC/ |
| SEQ ID NO: 77 | /52M O ErG/*/12M OErA/*/12M OErC/*/12MOE rT/*/iM e-dC/*T*A */iM e- |
| | dC/*A*/iM e-dC/*T*G*T*/iM e-dC/*/iM e- |
| | dC/*A*/12MOErG/*/12MOErC/*/12MOErC/*/32MOErA/ |
| SEQ ID NO: 78 | /52M O E rC/*/12M O E rT/*/i2M OErC/*/12MOErC/*A*T*T*A*G*/iMe- |
| | dC/*T*/iM e-dC/*/iM e-dC/*T*/iM e- |
| | dC/*A*/12MOErG/*/12MOErA/*/12M OErG/*/32MOErT/ |
| SEQ ID NO: 79 | /52M O E rT/*/12M O ErC/*/i2M O ErC/*/i2MOErT/*/iM e-dC/*/iM e- |
| | dC/*T*A*A*/iM e-dC/*/iM e-dC/*T*/iM e-dC/*T*T*/iM e- |
| | dC/*/12MOErC/*/12MOErA/*/12MOErG/*/32M OErA/ |
| SEQ ID NO: 80 | /52M O E rC/*/12M OErC/*/12MOErA/*/12MOErC/*A*T*/IM e- |
| | dC/*T*/iM e-dC/*A*G*/iM e-dC/*/iM e- |
| | dC/*A*T*T*/12MOErC/*/12MOErC/*/12MOErT/*/32MOErT/ |
| SEQ ID NO: 101 | /52M OErG/*/i2M OErG/*/12MOErA/*/12MOErG/*G*G*A*T*G*A*G* |
| | G*A*T*/iM e-dC/*A*/12M O ErC/*/12MOErA/*/12MOErG/*/32MOErA/ |
| SEQ ID NO: 102 | /52M OErG/*/12M OErC/*/i2M OErT/*/12MOErT/*G*/iM e-dC/*T*/iM e- |
| | dC/*/iM e-dC/*T*T*T*/IM e- |
| | dC/*T*T*G*/12MOErG/*/12MOErA/*/12MOErG/*/32MOErG/ |
| SEQ ID NO: 103 | /52M OErT/*/12MOErA/*/12MOE rT/*/12M OErC/*T*/iM e- |
| | dC/*A*G*A*G*/IM e- |
| | dC/*A*G*G*A*G*/12MOErT/*/12MOErT/*/12MOErG/*/32MOErT/ |
| SEQ ID NO: 104 | /52M O ErG/*/12M OErC/*/12M OErT/*/12MOErC/*T*G*T*A*/iMe- |
| | dC/*/iM e-dC/*A*A*T*G*/iM e-dC/*/iM e- |
| | dC/*/12MOErT/*/12MOErC/*/12MOErA/*/32M OErG/ |
| SEQ ID NO: 105 | /52M OErC/*/12MOErA/*/12M O ErG/*/12MOErA/*A*/iM e- |
| | dC/*A*T*G*/IM e-dC/*A*G*/iM e- |
| | dC/*T*T*T*/12MOErT/*/12MOErT/*/12MOErC/*/32MOErC/ |
| SEQ ID NO: 106 | /52M OErG/*/12M OErC/*/12M OErC/*/i2MOErA/*T*T*T*/iMe- |
| | dC/*/iM e- |
| | dC/*A*G*A*T*A*T*T*/12MOErC/*/12MOErA/*/12MOErG/*/32MOErG/ |
| SEQ ID NO: 107 | /52M OErT/*/i2M OErC/*/12M OErA/*/12MOErG/*T*T*T*T*/iM e- |
| | dC/*/iM e-dC/*T*T*G*G*G*/iM e- |
| | dC/*/12MOErT/*/i2M OErG/*/i2M OErC/*/32M OErA/ |
| SEQ ID NO: 108 | /52M OErG/*/12MOErT/*/12MOET/*/12MOErG/*/iM e- |
| | dC/*T*G*A*A*A*T*G*T*/IM e-dC/*T*/iMe- |
| | dC/*/12MOErC/*/12MOErA/*/12MOErT/*/32MOErC/ |
| SEQ ID NO: 109 | /52M OErC/*/12MO ErC/*/12MOErC/*/12MOErT/*/iM e-dC/*/iM e- |
| | dC/*/iM e-dC/*A*/iM e-dC/*T*A*/iM e- |
| | dC/*A*T*T*T*/12MOErG/*/12MOErC/*/12MOErA/*/32MOErT/ |
| SEQ ID NO: 110 | /52M OErC/*/12MOErT/*/12MOErA/*/12MOErG/*A*A*/iM e-dC/*/iM e- |
| | dC/*T*/iM e-dC/*A*T*T*/iM e- |
| | dC/*A*G*/12MOErT/*/12M OErG/*/12MOErG/*/32MOErT/ |
| SEQ ID NO: 111 | /52M OErG/*/i2M OErA/*/i2M OErT/*/12M OErT/*/iM e-dC/*A *A */iM e- |
| | dC/*T*G*/iM e-dC/*T*G*T*/iM e-dC/*/iM e- |
| | dC/*/12MOErT/*/12MOErT/*/i2M OErG/*/32MOErA/ |
| SEQ ID NO: 112 | /52M OErC/*/12M OErC/*/12MOErA/*/12MOErC/*A*T*A*/iM e- |
| | dC/*A*A*/iM e-dC/*T*G*/iM e- |
| | dC/*T*T*/12MOErC/*/12MOErT/*/12MOErT/*/32MOErC/ |
| SEQ ID NO: 113 | /52M OErC/*/12M O ErC/*/12MOErA/*/12MOErG/*A*/iM e-dC/*/iM e- |
| | dC/*/iM e-dC/*A*G*T*A*/IM e- |
| | dC/*T*A*T*/12MOErG/*/12MOErC/*/12MOErC/*/32MOErA/ |
| SEQ ID NO: 114 | /52M OErT/*/i2M OErT/*/12M O ErC/*/12MOErC/*/iM e- |
| | dC/*A*G*A*A*/iM e-dC/*T*/iM e-dC/*/iM e-dC/*/iM e- |
| | dC/*T*A*/12MOErA/*/12MOErT/*/12MOErC/*/32MOErA/ |
| SEQ ID NO: 115 | /52M OErG/*/12M O ErG/*/12MOErT/*/12M OErA/*A*/iM e-dC/*/iM e- |
| | dC/*T*T*T*/IM e- |
| | dC/*T*G*T*G*T*/12MOErC/*/12MOErT/*/i2MOErG/*/32MOErG/ |
| SEQ ID NO: 116 | /52M OErG/*/12M OErG/*/i2M OErC/*/12M OErC/*T*T*/iM e- |
| | dC/*A*A*/IM e-dC/*A*A*T*/iM e-dC/*T*/iM e- |
| | dC/*/12MOErT/*/12M OErC/*/12MOErT/*/32MOErT/ |
| SEQ ID NO: 117 | /52M OErG/*/i2M OErC/*/12M OErC/*/12MOET/*T*T*T*T*G*T*A*/I |
| | M e-dC/*T*G*G*G*/12MOErA/*/12MOErC/*/12MOErA/*/32MOErC/ |
| SEQ ID NO: 118 | /52M OErT/*/12MOErC/*/12M OErT/*/i2M OErG/*/iM e- |
| | dC/*T*G*T*T*/iM e-dC/*/iM e-dC/*A*G*/iM e-dC/*/iM e-dC/*/iM e- |
| | dC/*/12MOErA/*/12M OErC/*/12MOErA/*/32MOErT/ |
| SEQ ID NO: 119 | /52M OErA/*/12MOErT/*/12M O ErC/*/12MOErT/*G*/iM e- |
| | dC/*T*G*T*T*/iM e-dC/*/iM e-dC/*A*G*/iM e-dC/*/iM e- |
| | dC/*/12MOErC/*/12MOErA/*/12MOErC/*/32MOErA/ |
| SEQ ID NO: 120 | /52M OErC/*/12MOE rT/*/12M OErA/*/12MOErA/*A*G*T*T*/iMe- |
| | dC/*T*G*A*G*G*G*/IM e- |
| | dC/*/12MOErT/*/12M OErG/*/12M OErC/*/32MOErA/ |
| SEQ ID NO: 121 | /52M OErC/*/12MOErA/*/12MOErT/*/12MOErA/*/iM e- |
| | dC/*T*G*T*G*G*/iMe- |
| | dC/*A*T*G*A*G*/12MOErT/*/12MOErT/*/12MOErG/*/32MOErT/ |
| SEQ ID NO: 122 | /52M OErG/*/i2MOErA/*/12M O ErC/*/12M OErT/*A*/iM e-dC/*/iM e- |
| | dC/*A*T*T*T*/iMe- |
| | dC/*A*T*T*T*/12MOErG/*/12MOErG/*/12MOErC/*/32MOErC/ |
| SEQ ID NO: 123 | /52M OErC/*/12MOErA/*/12MOErT/*/12MOErT/*T*/iM e-dC/*/iM e- |
| | dC/*A*G*G*T*/iM e-dC/*A*G*/iM e- |
| | dC/*T*/12MOErT/*/12MOErA/*/12MOErC/*/32MOErT/ |
| SEQ ID NO: 124 | /52M OErC/*/12M OErA/*/12MOErC/*/12MOErC/*A*A*G*G*/iM e- |
| | dC/*A*/IM e-dC/*A*A*G*/iM e- |
| | dC/*T*/12M OErC/*/12MOErA/*/12MOErG/*/32MOErC/ |
| SEQ ID NO: 125 | /52M OErA/*/12MOErA/*/12M OErA/*/12M O ErG/*/iM e-dC/*T*G*/iM e- |
| | dC/*A*T*T*T*T*T*/iMe-dC/*/iM e- |
| | dC/*/12MOErT/*/i2M OErG/*/12MOErC/*/32MOErC/ |
| SEQ ID NO: 126 | /52M OErA/*/12M O E rC/*/12M OErA/*/12MOErG/*T*G*T*T*/iM e- |
| | dC/*T*A*A*A*G*G*/IM e- |
| | dC/*/12MOErT/*/12MOErG/*/12MOErG/*/32MOErC/ |
| SEQ ID NO: 127 | /52M OErC/*/12MOErA/*/12M O ErG/*/12MOErA/*/iM e-dC/*A */iM e- |
| | dC/*A*T*/iM e-dC/*A*T*/iM e- |
| | dC/*A*G*G*/12MOErG/*/12MOErC/*/12MOErC/*/32MOErT/ |
| SEQ ID NO: 128 | /52M OErA/*/12M OErC/*/12M OErA/*/12MOErG/*A*/iM e-dC/*A*/iM e- |
| | dC/*A*T*/iM e-dC/*A*T*/iM e- |
| | dC/*A*G*/12MOErG/*/12MOErG/*/12MOErC/*/32MOErC/ |
| SEQ ID NO: 129 | /52M OErC/*/12M OErA/*/12M OErC/*/12MOErA/*G*A*/IM e- |
| | dC/*A*/iM e-dC/*A*T*/iM e-dC/*A*T*/iM e- |
| | dC/*A*/12MOErG/*/12MOErG/*/12MOErG/*/32MOErC/ |
| SEQ ID NO: 130 | /52M OErG/*/12M OErA/*/12M O ErC/*/12M OErT/*/iM e- |
| | dC/*A*G*G*G*A*T*G*G*G*/IM e- |
| | dC/*T*/12MOErC/*/12MOErT/*/12MOErT/*/32MOErC/ |
| SEQ ID NO: 131 | /52M OErG/*/12M OErG/*/12M OErA/*/12M OErC/*T*/iM e- |
| | dC/*A*G*G*G*A*T*G*G*G*/IM e- |
| | dC/*/12MO ErT/*/i2M OErC/*/12MOErT/*/32MOErT/ |
| SEQ ID NO: 132 | /52M OErT/*/12M OErG/*/12M OErG/*/12MOErA/*/iM e-dC/*T*/iM e- |
| | dC/*A*G*G*G*A*T*G*G*G*/12MOErC/*/12MOErT/*/12MOErC/*/32 |
| | MOET/ |
| SEQ ID NO: 133 | /52M OErT/*/12M OErC/*/12M OErC/*/12M OErC/*T*T*/iM e-dC/*/iM e- |
| | dC/*T*T*/iM e-dC/*/iM e-dC/*A*T*/iM e- |
| | dC/*T*/12MOErT/*/12MOErT/*/12MOErC/*/32MOErT/ |
| SEQ ID NO: 134 | /52M OErC/*/12M OErT/*/12M O ErC/*/12M O ErC/*/iM e-dC/*T*T*/iM e- |
| | dC/*/iM e-dC/*T*T*/iM e-dC/*/iM e-dC/*A*T*/iM e- |
| | dC/*/12MOErT/*/12MOErT/*/i2M OErT/*/32MOErC/ |
| SEQ ID NO: 135 | /52M OErA/*/12M O E rC/*/12M OErA/*/12MOErT/*A*/IM e- |
| | dC/*T*G*T*G*G*/iMe- |
| | dC/*A*T*G*A*/12MOErG/*/12MOErT/*/12MOErT/*/32MOErG/ |
| SEQ ID NO: 136 | /52M OErC/*/i2M OErA/*/12MOErA/*/12MOErT/*/iM e- |
| | dC/*A*G*A*G*T*A*A*A*/IM e- |
| | dC/*T*G*/12MOErA/*/12M OErC/*/12MOErC/*/32M OErC/ |
| SEQ ID NO: 137 | /52M OErG/*/12MOErA/*/12M O ErC/*/12MOErA/*G*G*A*A*G*/iMe- |
| | dC/*A*/iM e- |
| | dC/*A*A*A*A*/12MOErC/*/12MOErT/*/12MOErC/*/32MOErA/ |
| SEQ ID NO: 138 | /52M OErG/*/12MOErG/*/12M OErA/*/12MOErC/*A*A*G*T*G*/iMe- |
| | dC/*A*T*/iM e-dC/*A*T*/iM e- |
| | dC/*/12MOErT/*/12MOErA/*/12MOErT/*/32M OErG/ |
| SEQ ID NO: 139 | /52M OErT/*/12MOErA/*/12MOErA/*/12MOErA/*T*A*G*/iMe- |
| | dC/*/iM e-dC/*A*G*A*/iM e-dC/*/iM e-dC/*/iM e- |
| | dC/*A*/12MOErG/*/12MOErT/*/12MOErA/*/32MOErC/ |
| SEQ ID NO: 140 | /52M OErG/*/12M OErG//12M OErA//12MOErT/*T*/IM e- |
| | dC/*A*A*/IM e-dC/*T*G*/iM e-dC/*T*G*T*/iM e- |
| | dC/*/12MOErC//12MOErT//12MOErT/*/32MOErG/ |
| SEQ ID NO: 141 | /52M OErG//12MOErG/*/12MOErA//12MOErT/*T*/iMe- |
| | dC/*A*A*/iM e-dC/*T*G*/iM e-dC/*T*G*T*/IM e- |
| | dC/*/12M OErC//12MOET/*/12MOErT/*/32MOErG/ |
| SEQ ID NO: 142 | /52M OErG/*/12M O ErG//12M O ErA//i2M OErT/T*/iM e-dC/*A *A */iM e- |
| | dC/*T*G*/iM e-dC/*T*G*T*/iM e-dC/*/12MOErC/*/12MOErT/ |
| | /12MOErT/*/32M OErG/ |
| SEQ ID NO: 143 | /52M OErA/*/12MO ErA//i2M OErC//12M OErC/*T*T*T*/iMe- |
| | dC/*T*G*T*G*T*/iM e-dC/*T*G*/12MOErG//12MOErG/ |
| | /12M OErC/*/32M OErC/ |
| SEQ ID NO: 144 | /52M OErA//i2M OErA//12M O E rC/*/12M OErC/*T*T*T*/iM e- |
| | dC/*T*G*T*G*T*/iMe-dC/*T*G*/12MOErG//i2MOErG/ |
| | /12MOErC/*/32MOErC/ |
| SEQ ID NO: 145 | /52M OErA/*/12MOErA//12MOErC//12M OErC/T*T*T*/iMe- |
| | dC/*T*G*T*G*T*/iMe-dC/*T*G*/12MOErG//12MOErG/ |
| | /12MOErC/*/32MOErC/ |
| SEQ ID NO: 146 | /52M OErG/*/12M O E rC//i2MO E rT//12MOErT/*G*/iM e-dC/*T*/iM e- |
| | dC/*/iM e-dC/*T*T*T*/iM e-dC/*T*T*G*/12MOErG//12MOErA/ |
| | /12M OErG/*/32M OErG/ |
| SEQ ID NO: 147 | /52M OErG//12MOErC//12M OErT//12MOErT/*G*/iM e-dC/*T*/iM e- |
| | dC/*/iM e-dC/*T*T*T*/iM e-dC/*T*T*G*/12MOErG/*/12MOErA/ |
| | /12M OErG/*/32MOErG/ |
| SEQ ID NO: 148 | /52M OErG/*/i2M O ErC//i2M O ErT//12MOErT/G*/iM e-dC/*T*/iM e- |
| | dC/*/iM e-dC/*T*T*T*/iM e-dC/*T*T*G*/12M OErG/*/12MOErA/ |
| | /12M OErG/*/32MOErG/ |
| SEQ ID NO: 149 | /52M OErG//12M OErG//12MOE rT/*/12MOErA/*A */iM e-dC/*/iM e- |
| | dC/*T*T*T*/iM e-dC/*T*G*T*G*T*/12MOErC//12MOET/ |
| | /12M OErG/*/32M OErG/ |
| SEQ ID NO: 150 | /52M OErG/*/12MOErG//12MO E rT//12MOErA/*A */iM e-dC/*/iM e- |
| | dC/*T*T*T*/iMe-dC/*T*G*T*G*T*/12MOErC/*/12MOET/ |
| | /12M OErG/*/32MOErG/ |
| SEQ ID NO: 151 | /52M OErG/*/12M O ErG//12MOErT//i2M OErA/A */iM e-dC/*/iM e- |
| | dC/*T*T*T*/iM e-dC/*T*G*T*G*T*/12MOErC//12MOErT/ |
| | /12M OErG/*/32M OErG/ |
| SEQ ID NO: 152 | /52M OErG/*/12M O ErG//12MOErC//12MOErC/*T*T*/IM e- |
| | dC/*A*A*/IM e-dC/*A*A*T*/iM e-dC/*T*/iM e- |
| | dC/*/12MOErT/*/12M OErC//12MOErT/*/32MOErT/ |
| SEQ ID NO: 153 | /52M OErG/*/12M OErG//i2M OE rC//12M OErC/*T*T*/IM e- |
| | dC/*A*A*/iM e-dC/*A*A*T*/iM e-dC/*T*/iM e- |
| | dC/*/12MOErT//i2M OErC//12MOErT/*/32MOErT/ |
| SEQ ID NO: 154 | /52M OErG/*/12M O ErG//12M O ErC//12M OErC/T*T*/iM e- |
| | dC/*A*A*/iM e-dC/*A*A*T*/iM e-dC/*T*/iM e- |
| | dC/*/12MOErT/*/12MOErC//12MOErT/*/32MOErT/ |
| SEQ ID NO: 155 | 52M OErG/*/12M O ErC//i2M OErA//12MOErA/*T*/iM e- |
| | dC/*T*G*G*T*G*T*A*G*A*/IM e-dC/*/12MOErC/*/12MOErC/ |
| | /12M OErT/*/32MOErT/ |
| SEQ ID NO: 156 | /52M O ErG/*/i2M O E rC//i2M OErA//12M OErA/*T*/iM e- |
| | dC/*T*G*G*T*G*T*A*G*A*/IM e-dC/*/12MOErC//12MOErC/ |
| | /12MOErT/*/32MOErT/ |
| SEQ ID NO: 157 | /52M OErG//12M O E rC//12M OErA//i2M OErA/*T*/iM e- |
| | dC/*T*G*G*T*G*T*A*G*A*/IM e-dC/*/12MOErC/*/12MOErC/ |
| | /12M OErT/*/32MOErT/ |
| SEQ ID NO: 158 | /52M OErG/*/i2M O ErG//12M O ErG//12MOErA/*T*G*G*G*/IM e- |
| | dC/*T*/iM e-dC/*T*T*/iM e-dC/*A*T*/12MOErC/*/i2MOErA/ |
| | /12M OErT/*/32M OErC/ |
| SEQ ID NO: 159 | /52M OErG/*/i2M O ErG//12M OErG//i2M OErA/*T*G*G*G*/iM e- |
| | dC/*T*/iM e-dC/*T*T*/iM e-dC/*A*T*/12MOErC//12MOErA/ |
| | /12M OErT/*/32M OErC/ |
| SEQ ID NO: 160 | /52M O ErG//12M OErG/*/12M OErG//12MOErA/*T*G*G*G*/IM e- |
| | dC/*T*/iM e-dC/*T*T*/iM e-dC/*A*T*/12MOErC/*/12MOErA/ |
| | /12M OErT/*/32M OErC/ |
| SEQ ID NO: 161 | /52M OErA/*/12M O E rC//i2M O ErC//12MOErA/*A*G*T*T*/iMe- |
| | dC/*A*G*T*T*T*/iM e-dC/*/iM e-dC/*/12MOErA/*/12MOErG/ |
| | /12M OErG/*/32M OErG/ |
| SEQ ID NO: 162 | /52M OErA/*/i2M O ErC//i2M OErC//12MOErA/*A*G*T*T*/IM e- |
| | dC/*A*G*T*T*T*/iMe-dC/*/iM e-dC/*/12MOErA//12MOErG/ |
| | /12M OErG/*/32M OErG/ |
| SEQ ID NO: 163 | /52M OErA//12M OErC//i2M OErC/*/12MOErA/*A*G*T*T*/iM e- |
| | dC/*A*G*T*T*T*/iM e-dC/*/iM e-dC/*/12MOErA/*/12M OErG/ |
| | /12M OErG/*/32M OErG/ |
| SEQ ID NO: 164 | /52M OErG/*/12M O ErG//i2M O ErA//12MOErT/*T*/iM e- |
| | dC/*A*A*/iM e-dC/*T*G*/iM e-dC/*T*G*T*/iM e- |
| | dC/*/12MOErC/*/12MOErT//12MOErT/*/32MOErG/ |
| SEQ ID NO: 165 | /52M OErG//12MOErG//12MOErA/*/12MO ErT/*T*/iM e-dC/*A *A */iM e- |
| | dC/*T*G*/iM e-dC/*T*G*T*/iM e-dC/*/12M OErC//i2MOErT/ |
| | /12M OErT/*/32MOErG/ |
| SEQ ID NO: 166 | /52M OErA/*/12MOErT//12MO E rT//12MOErT/*/iM e-dC/*/iM e- |
| | dC/*T*/iM e-dC/*/iM e-dC/*A*/iM e-dC/*A*A*/iM e-dC/*/iM e- |
| | dC/*A*/12M OErG/*/12MOErC//12MOErT/*/32MOErG/ |
| SEQ ID NO: 167 | /52M OErA/*/12MO E rT//12MO E rT//12MOErT/*/iM e-dC/*/iM e- |
| | dC/*T*/iM e-dC/*/iM e-dC/*A*/iM e-dC/*A*A*/iM e-dC/*/iM e- |
| | dC/*A*/12MOErG//i2M OErC//12MOErT/*/32M OErG/ |
| SEQ ID NO: 168 | /52M OErA//i2M OErT//12M OErT/*/i2M OErT/*/iM e-dC/*/iM e- |
| | dC/*T*/iM e-dC/*/iM e-dC/*A*/iM e-dC/*A*A*/iM e-dC/*/iM e- |
| | dC/*A*/12MOErG/*/12MOErC//12MOErT/*/32MOErG/ |
| SEQ ID NO: 169 | /52M OErC/*/12M OErA//12M OErG//12M OErC/*/iM e- |
| | dC/*T*T*T*T*T*G*T*A*/iMe-dC/*T*G*/12MOErG/*/12MOErG/ |
| | /12M OErA/*/32MOErC/ |
| SEQ ID NO: 170 | /52M OErC//12MO ErA//12M OErG/*/12M OErC/*/iM e- |
| | dC/******* T*T*G*T*A*/iMe-dC/*T*G*/12MOErG/12MOErG/ |
| | /12MOErA/*/32MOErC/ |
| SEQ ID NO: 171 | /52M OErC/*/12MOErA//12M O ErG//i2M OE rC/*/iM e- |
| | dC/*T*T*T*T*T*G*T*A*/iMe-dC/*T*G*/12MOErG//12MOErG/ |
| | /12MOErA/*/32MOErC/ |
| SEQ ID NO: 172 | /52M OErG/*/i2M O E rC//i2M OErT//12MOErT/*G*/iM e-dC/*T*/iM e- |
| | dC/*/iM e-dC/*T *** T*/iMe-dC/*T*T*G*/12MOErG/*/12MOErA/ |
| | /12M OErG/*/32M O ErG/ |
| SEQ ID NO: 173 | /52M OErG//12M OErC/*/12M OErT//12MOErT/*G*/iM e-dC/*T*/iM e- |
| | dC/*/iM e-dC/*T*T*T*/iM e-dC/*T*T*G*/12MOErG/*/12MOErA/ |
| | /12M OErG/*/32MOErG/ |
| SEQ ID NO: 174 | /52M OErG/*/i2M O E rC//12M OErC//12MOErA/*T*T*T*/iM e-dC/*/iM e- |
| | dC/*A*G*A*T*A*T*T*/12MOErC/*/12MOErA/ |
| | /12M OErG/*/32MOErG/ |
| SEQ ID NO: 175 | /52M OErG//i2M OE rC//12M OErC/*/12MOErA/*T*T*T*/iM e-dC/*/iM e- |
| | dC/*A*G*A*T*A*T*T*/12MOErC/*/12MOErA/ |
| | /12M OErG/*/32MOErG/ |
| SEQ ID NO: 176 | /52M OErG/*/i2M OErC//12M O ErC//i2MOErA/*T*T*T*/iM e-dC/*/iM e- |
| | dC/*A*G*A*T*A*T*T*/12MOErC//i2MOErA//12MOErG/*/32MOErG/ |
| SEQ ID NO: 177 | /52M OErG//12M O ErG/*/12M OErC//12M OErC/*T*T*/iM e- |
| | dC/*A*A*/iM e-dC/*A*A*T*/iM e-dC/*T*/iM e- |
| | dC/*/12MOErT/*/12MOErC//12MOErT/*/32MOErT/ |
| SEQ ID NO: 178 | /52M OErG/*/12M O E rC//i2M OErC/ |
| | /12M OErT/*T*T*T*T*G*T*A*C*T*G*G*G*/12MOErA/*/12MOErC/ |
| | /12MOErA/*/32M OErC/ |
| SEQ ID NO: 179 | /52M OErG//i2M OErC/*/12M O ErC/ |
| | /12M OErT/*T*T*T*T*G*T*A*C*T*G*G*G*/12MOErA/*/2MOErC/ |
| | /12M OErA/*/32M OErC/ |
| SEQ ID NO: 180 | /52M OErG/*/i2M O ErC//12M OErC/ |
| | /12M OErT/*T*T*T*T*G*T*A*C*T*G*G*G*/12MOErA//12MOErC/ |
| | /12M OErA/*/32M OErC/ |
| SEQ ID NO: 181 | /52M OErG/*/i2M O ErA//12M O E rC//12MOErT/*A */iM e-dC/*/iM e- |
| | dC/*A*T*T*T*/iMe-dC/*A*T*T*T*/12MOErG/*/12MOErG/ |
| | /12M OErC/*/32M OErC/ |
| SEQ ID NO: 182 | /52M OErG//i2M OErA/*/12M O ErC//i2M OErT/*A*/iM e-dC/*/iM e- |
| | dC/*A*T*T*T*/iMe-dC/*A*T*T*T*/12MOErG/*/12MOErG/ |
| | /12M OErC/*/32M OErC/ |
| SEQ ID NO: 183 | /52M OErG/*/i2M O ErA//12M O E rC//12M OErT/*A */iM e-dC/*/iM e- |
| | dC/*A*T*T*T*/iMe-dC/*A*T*T*T*/12MOErG//12MOErG/ |
| | /12M OErC/*/32MOErC/ |
| SEQ ID NO: 204 | 52M OErC/*/12MOErC//12M OErT//12MOErT/*T*/iM e- |
| | dC/*T*T*G*G*A*G*G*G*A*T*/12MOErG/*/12MOErA/ |
| | /12M OErG/*/32M OErG/ |
| SEQ ID NO: 205 | /52M OErC/*/12MOErC//12MOErT//12MOErT/*T*/iM e- |
| | dC/*T*T*G*G*A*G*G*G*A*T*/12MOErG//12MOErA/ |
| | /12M OErG/*/32M OErG/ |
| SEQ ID NO: 206 | /52M OErC/*/12MOErC//12MOErT//i2MOErT/T*/iM e- |
| | dC/*T*T*G*G*A*G*G*G*A*T*/12MOErG/*/12MOErA/ |
| | /12M OErG/*/32M OErG/ |
| SEQ ID NO: 207 | /52M OErA/*/i2M O E rC//i2M OErA//12MOErG/*G*T*G*/iMe- |
| | dC/*T*/iM e-dC/*T*G*T*/iM e-dC/*T*G*/12MOErT/*/12MOErG/ |
| | /12M OErC/*/32M OErC/ |
| SEQ ID NO: 208 | 52M OErA/*/12MOErC//i2M OErA//12M OErG/*G*T*G*/IM e- |
| | dC/*T*/iM e-dC/*T*G*T*/iM e-dC/*T*G*/12MOErT//12MOErG/ |
| | /12M OErC/*/32M OErC/ |
| SEQ ID NO: 209 | /52M OErA/*/i2M O ErC//i2M OErA//12MOErG/G*T*G*/iM e- |
| | dC/*T*/iM e-dC/*T*G*T*/iM e-dC/*T*G*/12MOErT/*/12MOErG/ |
| | /12M OErC/*/32MOErC |
| SEQ ID NO: 210 | /52M OErA/*/12M OE rC//12M O ErC//12MOErT/*T*T*/IM e- |
| | dC/*T*G*T*G*T*/iMe-dC/*T*G*G*/12MOErG//12MOErC/ |
| | /12M OErC/*/32MOErA/ |
| SEQ ID NO: 211 | /52M OErA/*/i2M O E rC//12M O ErC//12MOErT/*T*T*/IM e- |
| | dC/*T*G*T*G*T*/iM e-dC/*T*G*G*/12MOErG/*/12MOErC/ |
| | /12M OErC/*/32M OErA/ |
| SEQ ID NO: 212 | /52M OErA/*/12M O ErC//12M O ErC//12MOErT/T*T*/IM e- |
| | dC/*T*G*T*G*T*/iM e-dC/*T*G*G*/12MOErG/*/12MOErC/ |
| | /12M OErC/*/32MOErA/ |
| SEQ ID NO: 213 | /52M OErA/*/12M O ErC//12M O ErA//12M OErG/*/iM e-dC/*/iM e- |
| | dC/*T*T*T*T*T*G*T*A*/iMe-dC/*T*/12MOErG//i2MOErG/ |
| | /12M OErG/*/32M OErA/ |
| SEQ ID NO: 214 | /52M OErA/*/12M O ErC//12M OErA//i2M O ErG/*/iM e-dC/*/iM e- |
| | dC/*T*T*T*T*T*G*T*A*/iMe-dC/*T*/12MOErG/*/i2MOErG/ |
| | /12M OErG/*/32M OErA/ |
| SEQ ID NO: 215 | /52M OErA/*/12M O ErC//12M OErA//12MOErG//iM e-dC/*/iM e- |
| | dC/*T*T*T*T*T*G*T*A*/iMe-dC/*T*/12MOErG/*/12MOErG/ |
| | /12M OErG/*/32M O ErA/ |
| SEQ ID NO: 216 | /52M OErG/*/12M OErC//12M OErA//i2M OErC/*T*T*T*/iM e-dC/*/iM e- |
| | dC/*/iM e-dC/*/iM e-dC/*A*G*T*A*A*/12MOErA//12MOErC/ |
| | /12MOErT/*/32M OErT/ |
| SEQ ID NO: 217 | /52M OErG/*/12MO ErC//12MOErA//12MOErC/*T*T*T*/iM e-dC/*/iM e- |
| | dC/*/iM e-dC/*/iM e-dC/*A*G*T*A*A*/12MOErA/*/12MOErC/ |
| | /12M OErT/*/32MOErT/ |
| SEQ ID NO: 218 | /52M OErG/*/12M OErC//12M OErA//i2M OErC/T*T*T*/iM e-dC/*/iM e- |
| | dC/*/iM e-dC/*/iM e-dC/*A*G*T*A*A*/12MOErA/*/12MOErC/ |
| | /12M OErT/*/32MOErT/ |
| SEQ ID NO: 219 | /52M OErA/*/12M O ErC//12M OErA/*/12MOErG/*/iM e-dC/*/iM e- |
| | dC/*T*T*T*T*T*G*T*A*/iM e-dC/*T*/12MOErG/*/12MOErG/ |
| | /12M OErG/*/32M OErA/ |
| SEQ ID NO: 220 | /52M O ErA/*/12M OErA//12M O ErG//i2M O ErG//12MOErC/T/*/IM E- |
| | dC/*A*A*/iM e-dC/*/iM e-dC/*T*/iM e- |
| | dC/*A*A*/12MOErG//i2MOErC//12MOErA/*/12MOErG/*/32MOErT/ |
| SEQ ID NO: 221 | /52M O ErG/*/12M O ErG//i2M O E rC//12M OErT//12M OErC/A *A */iM e- |
| | dC/*/iM e-dC/*T*/iM e-dC/*A*A*G*/iM e- |
| | dC/*/12MOErA//12M OErG//12MOE rT/*/12MOErA/*/32MOErA/ |
| SEQ ID NO: 222 | /52M O ErG/*/12M O E rC//12M O E rT//12M O E rC//i2MOErA/A */IM e- |
| | dC/*/iM e-dC/*T*/iM e-dC/*A*A*G*/iM e- |
| | dC/*A*/2MOErG/T/12MOErA/*/12MOErA/*/32MOErT/ |
| SEQ ID NO: 223 | /52M O ErA/*/12M OErA//12M O ErT//i2M O ErA//12MO ErG//iM e- |
| | dC/*/iM e-dC/*A*G*A*/iM e-dC/*/iM e-dC/*/iM e- |
| | dC/*A*G*T/12MOErA//12M OErC*/12MOErT/*/32MOErA/ |
| SEQ ID NO: 224 | /52M O ErA/*/i2M O ErT//i2M OErA//12M O ErG//12M O E rC//iM e- |
| | dC/*A*G*A*/iM e-dC/*/iM e-dC/*/iM e- |
| | dC/*A*G*T*/12MOErA//12M OErC//12MOErT/*/12M OErA/*/32MOErT/ |
| SEQ ID NO: 225 | /52M O ErC/*/i2M OErA//i2M OErG//i2M OErA//12M OErC//iM e- |
| | dC/*/iM e-dC/*A*G*T*A*/iM e- |
| | dC/*T*A*T*/12MOErG//12MOErC//12MOErC*/i2MOErA/*/32M OErA/ |
| SEQ ID NO: 226 | /52M O ErC/*/i2M O ErC//12MOErA//12M O ErG//i2MOET/A */iM e- |
| | dC/*T*A*T*G*/IM e-dC/*/iM e- |
| | dC/*A*A*/12MOErT//12MOErC//12MOErA/*/12MOErG/*/32MOErA/ |
| SEQ ID NO: 227 | /52M O ErG/*/12M O E rT//12M O ErG//i2M O ErC//12MOErA/G*G*/iM e- |
| | dC/*T*T*/iM e- |
| | dC/*A*T*T*T*/12MOErC//i2MOErC//12MOErA/*/12MOErC/*/32MOErA/ |
| SEQ ID NO: 228 | /52M O ErT/*/12M O E rT//i2M O E rC//12MO ErC//12MOErA//iM e- |
| | dC/*A*G*/iM e-dC/*/iM e-dC/*/iM e-dC/*/i2M OErT/*/iM e- |
| | dC/*A*G*T//12MOErT//12MOErA/*/12MOErA/*/32M OErC/ |

Monomer Abbreviations
52MOEr = 5′ 2′-O-methoxyoethyl RNA
32MOEr = 3′ 2′-O-methoxyethyl RNA
i2MOEr = internal 2′-O-methoxyethyl RNA
iMe-dC = 5-methyl deoxycytidine
* = PS linkage
// = PO linkage (non-PS linkage)

Conjugation of the oligonucleotide 107 to one or more non-nucleotide moieties may improve the pharmacology of the oligonucleotide, e.g., by affecting the activity, cellular distribution, cellular uptake or stability of the oligonucleotide. In some embodiments the conjugate moiety can modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. The conjugate may also serve to reduce activity of the oligonucleotide in non-target cell types, tissues or organs, e.g., off target activity or activity in non-target cell types, tissues or organs.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g., bacterial toxins), vitamins, viral proteins (e.g., capsids) or combinations thereof.

Oligonucleotides 107 of the disclosure may be provided in pharmaceutical compositions that include any of the aforementioned oligonucleotides and/or oligonucleotide conjugates or salts thereof and a pharmaceutically acceptable diluent, carrier, salt and/or adjuvant. A pharmaceutically acceptable diluent includes A CSF (artificial cerebrospinal fluid) and pharmaceutically acceptable salts include, but are not limited to, sodium and potassium salts. In some embodiments the pharmaceutically acceptable diluent is sterile phosphate buffered saline or sterile sodium carbonate buffer. In some preferred embodiments, diluents for clinical application include Elliotts B solution and/or ACSF (artificial cerebrospinal fluid).

In some embodiments the oligonucleotide of the invention is in the form of a solution in the pharmaceutically acceptable diluent, for example dissolved in PBS or sodium carbonate buffer. The oligonucleotide may be pre-formulated in the solution or in some embodiments may be in the form of a dry powder (e.g., a lyophilized powder) which may be dissolved in the pharmaceutically acceptable diluent prior to administration. Suitably, for example the oligonucleotide may be dissolved in a concentration of 0.1-100 mg/mL, such as 1-10 mg/mL.

### EXAMPLES

The following examples provide exemplary methods for screening ASOs of the invention. In the examples, a series of ASOs were screened. Based on the resulting data, ASOs corresponding to SEQ ID NOS: 146, 155, 156, 158, 159, 161, 164, 169, 174, 175, 178, 179, 213, and 214 were identified as lead candidate ASOs based on dose-response efficacy, sequence motif liabilities, and off-target alignment analyses. Those ASOs showed the greatest in vitro efficacy, lowest off-target alignments, and limited sequence motif concerns. However, other ASOs as described herein also work as described to knock down UBE3A for the treatment of various conditions.

### Example 1—Single Dose Screening of UBE3A ASOs

Forty UBE3A-targeting ASOs (SEQ ID NOS: 1-40) were screened in vitro by treating primary fibroblasts, plated 10 k per well of a96-well plate, with 200 nM of ASO. ASOs were delivered by transfection using RNAiMax at 0.5 uL per well of a 96-well plate.

The data shown in FIG. 3 are qPCR data of normalized relative UBE3A transcript expression of ASO-treated fibroblasts versus a vehicle. All samples were normalized to a second vehicle condition. Cell only conditions (white) show no change in UBE3A expression. UBE3A siRNA was used as a positive control and shows ˜80% knockdown of UBE3A transcript. A non-targeting siRNA was used as a negative control and shows no knockdown of UBE3A. The top graph shows data for UBE3A ASOs 001-020 (SEQ ID NOS: 1-20). Bottom graph shows data for UBE3A ASOs 021-040 (SEQ ID NOS: 21-40).

All cells were transfected with ASOs 48-hours after plating. Cells were harvested for qPCR an additional 48 hours after ASO transfection. Actin was used as the normalizing gene for UBE3A. Each bar represents 3 technical replicates and 1 biological replicate. The dots above certain bars indicate preferred ASOs identified within this set of 40 ASOs, and correspond to SEQ ID NOS: 4, 7, 8, 14, 17, 18, 21, 26, 34, and 35.

FIG. 4 provides results showing the dose-response of ten ASO candidates (SEQ ID NOS.: 14, 17, 4, 7, 8, 18, 21, 26, 34, and 35) at 6 concentrations each, designed according to embodiments of the disclosure (about 20 bases in length with an about 10-12 base DNA central region flanked by RNA wings with 2′-0 modified RNA and phosphorothioate linkages throughout the ASO). All ten ASOs decreased UBE3A expression, relative to controls in a dose-dependent manner (vehicle-only treated cells and untreated “cells only” conditions).

### Example 2—Single Dose Screening of all UBE3A ASOs

Using the methods of Example 1, UBE3A-targeting ASOs (SEQ ID NOS: 1-80, 101-139, and 184-203) were screened in vitro by treating primary fibroblasts, plated 10 k per well of a 96-well plate, with 200 nM of ASO. ASOs were delivered by transfection using RNAiMax at 0.5 uL per well of a96-well plate.

All screened ASOs were designed according to embodiments of the disclosure, i.e., about 20 bases in length with an about 10-12 base DNA central region flanked by RNA wings with 2′-O modified RNA and phosphorothioate linkages throughout the ASO.

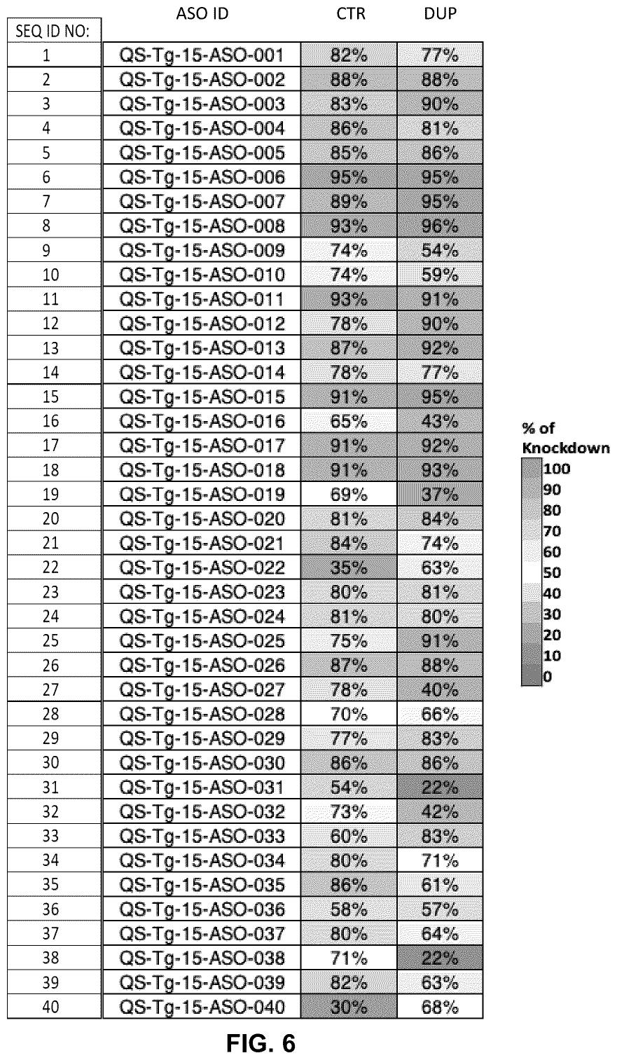

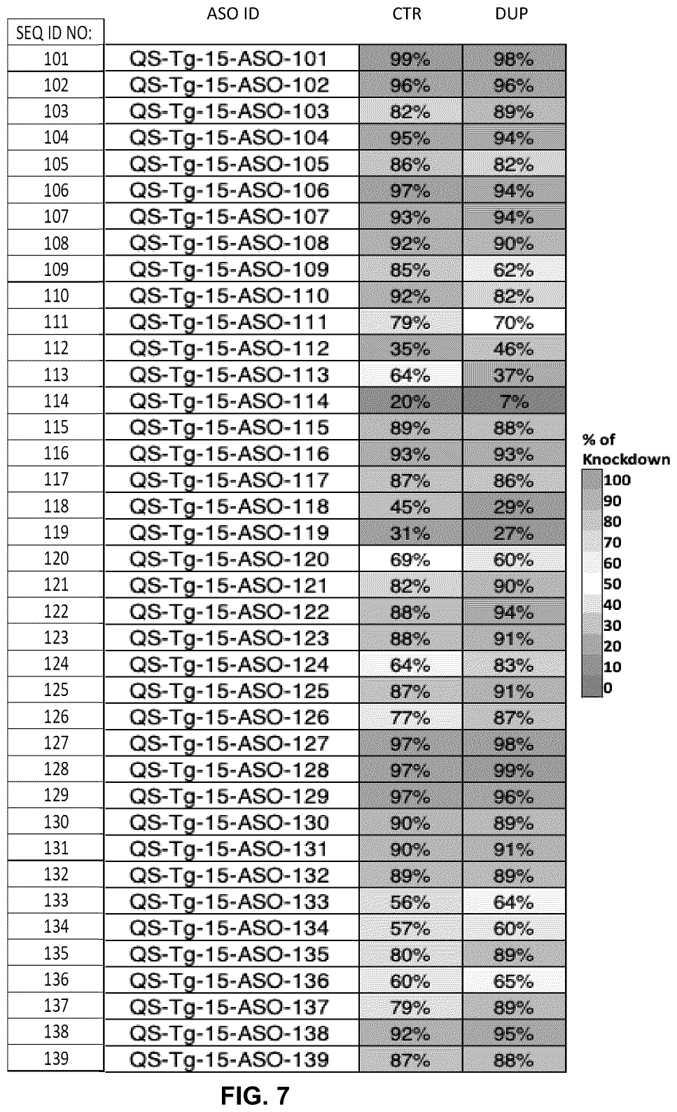

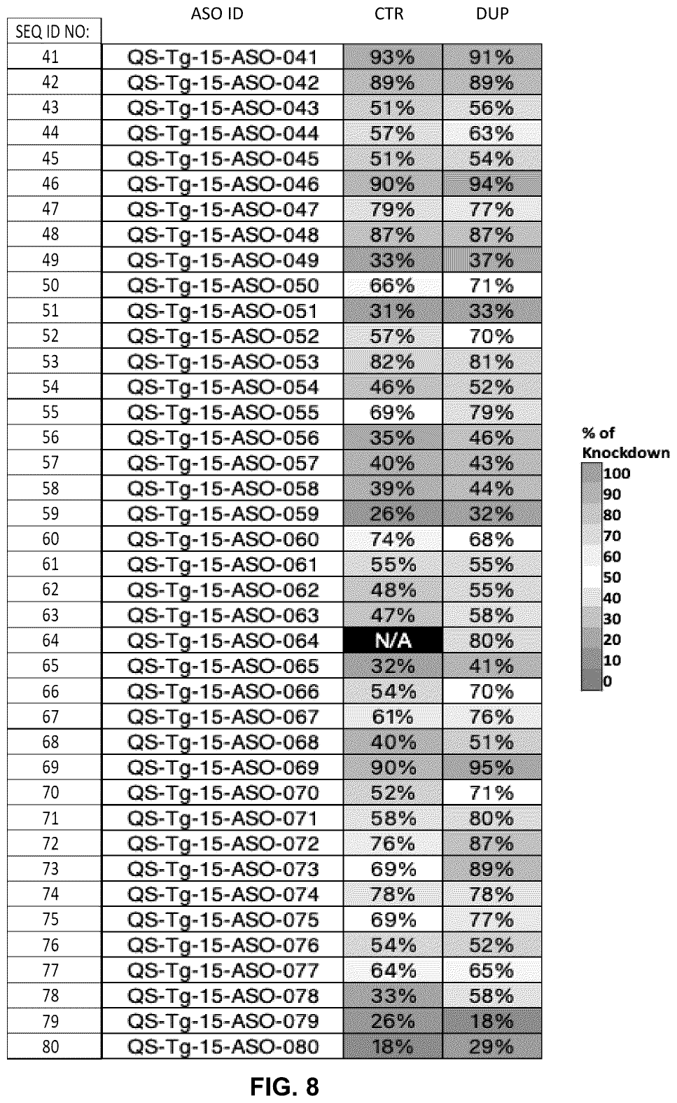

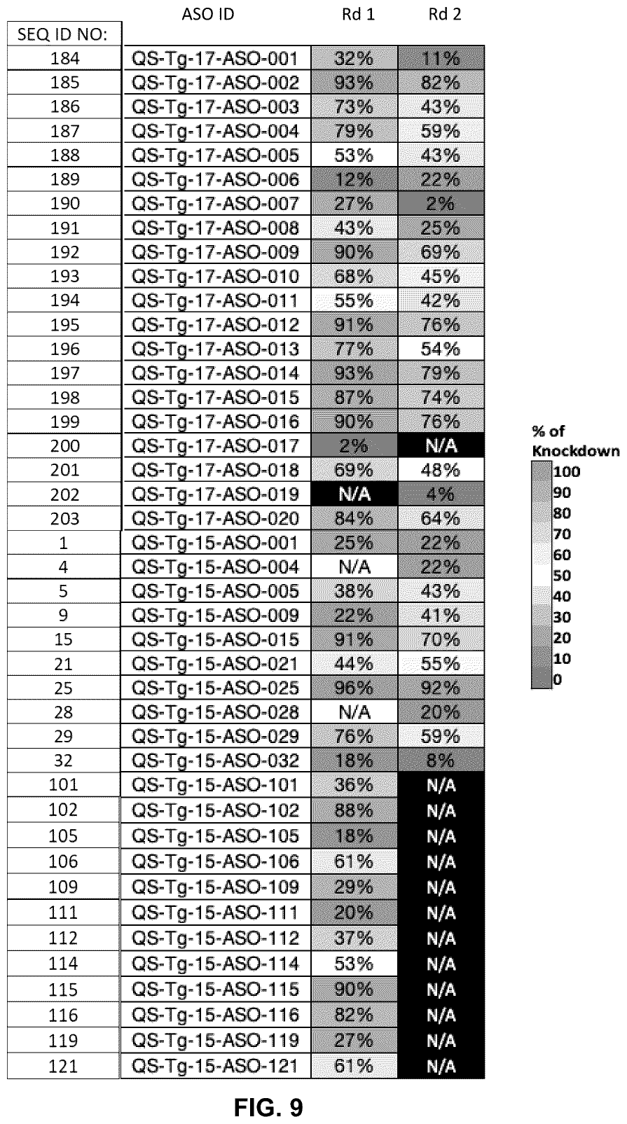

The data shown in FIGS. 6-9 are presented as summary tables of qPCR readouts of UBE3A knockdown (expressed as percent of UBE3A knockdown) for all 139 ASOs screened. All samples were normalized to either a vehicle condition or cell only condition. The tables of ASOs are broken down into UBE3A exon-targeting ASOs (FIGS. 6-7), UBE3A intron-targeting ASOs (FIG. 8), and UBE3A ASOs with 100% homology to both human and mouse UBE3A transcript (FIG. 9), for downstream rodent proof-of-concept in vivo studies.

All cells were transfected with ASOs 48-hours after plating. Cells were harvested for qPCR an additional 48 hours after ASO transfection. Actin was used as the normalizing gene for UBE3A. W here appropriate, ASOs were screened in both control fibroblasts and fibroblasts from a Dup15q patient (FIGS. 6-8). In FIG. 9, for the ASOs with mouse UBE3A homology, data is shown for 2 rounds.

### Example 3—Dose-Response Screening of UBE3A Lead ASO Candidates

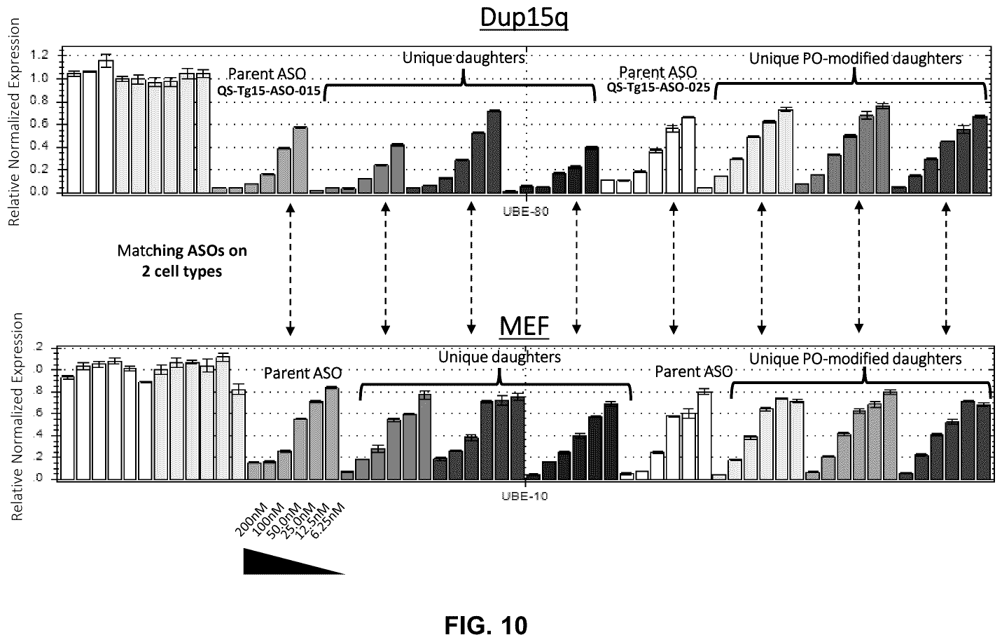

Based on the data from Examples 1 and 2, candidate lead UBE3A-targeting ASOs were selected based on greater than 80-85% transcript knockdown in the primary single-dose screenings. For each candidate lead, new ASOs with identical sequences, were synthesized with 1 to 3 phosphodiester (PO) backbone modifications each in the 3′ and 5′, 2′-MOE RNA-like wings, with total of 4-5 PO modifications (i.e., a PS linkage replaced with a PO linkage) per ASO. These modifications replace the corresponding PS linkages in the original lead ASOs. The PO-modified ASOs are referred to in FIG. 10 as daughter ASOs.

These candidate leads were then tested for dose-response modulation of UBE3A transcript expression. For these experiments either primary fibroblasts, plated 10 k per well of a 96-well plate, or mouse embryonic fibroblasts plated at 15 k per well, were plated onto a 96-well plate. ASOs were screened at 6 doses: 6.25, 12.5, 25, 50, 100, and 200 nM. ASOs were delivered by transfection using RNAiMax at 0.5 uL per well of a 96-well plate.

FIG. 10 displays example data of UBE3A ASO dose-response modulation of target expression for 2 lead candidate examples and their PO-modified daughter molecules in Dup15q patient fibroblasts (top) or mouse embryonic fibroblasts (bottom). All samples were normalized to vehicle conditions.

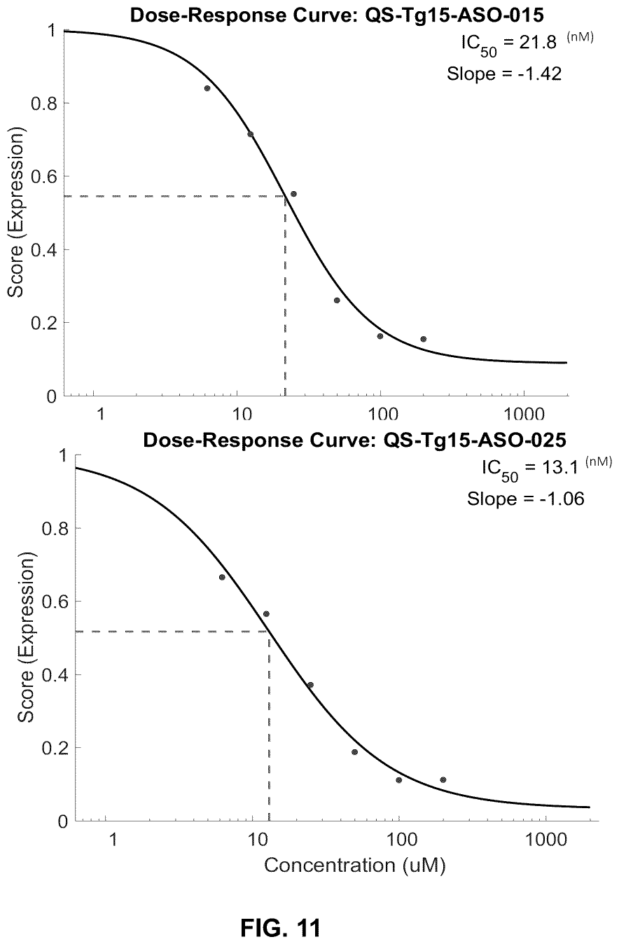

FIG. 11 plots the dose-response and indicates EC50 for the same 2 example lead candidates from FIG. 10. All cells were transfected with ASOs 48-hours after plating. Cells were harvested for qPCR an additional 48 hours after ASO transfection. Actin was used as the normalizing gene for UBE3A. Each data point represents 2 technical replicates and from 1 biological replicate.

### Example 4—Dose-Response Screening of UBE3A Lead ASO Candidates

Candidate lead UBE3A-targeting ASOs were selected based on greater than 80-85% transcript knockdown in the primary single-dose screening from Examples 1 and 2. For each candidate lead, new ASOs with identical sequences, were synthesized with 1 to 3 PO backbone modifications each in the 3′ and 5′, 2′-MOE RNA-like wings (total of 4-5 PO modifications per ASO), as described in Example 3. All candidate leads were then tested for dose-response modulation of UBE3A transcript expression.

For these experiments either primary fibroblasts, plated 10 k per well of a96-well plate, or mouse embryonic fibroblasts plated at 15 k per well, were plated onto a 96-well plate. ASOs were screened at 6 doses: 6.25, 12.5, 25, 50, 100, and 200 nM, unless otherwise indicated. ASOs were delivered by transfection using RNAiMax at 0.5 uL per well of a 96-well plate.

All samples were normalized to either vehicle or control conditions within each experiment. All cells were transfected with ASOs 48-hours after plating. Cells were harvested for qPCR an additional 48 hours after ASO transfection. Actin was used as the normalizing gene for UBE3A.

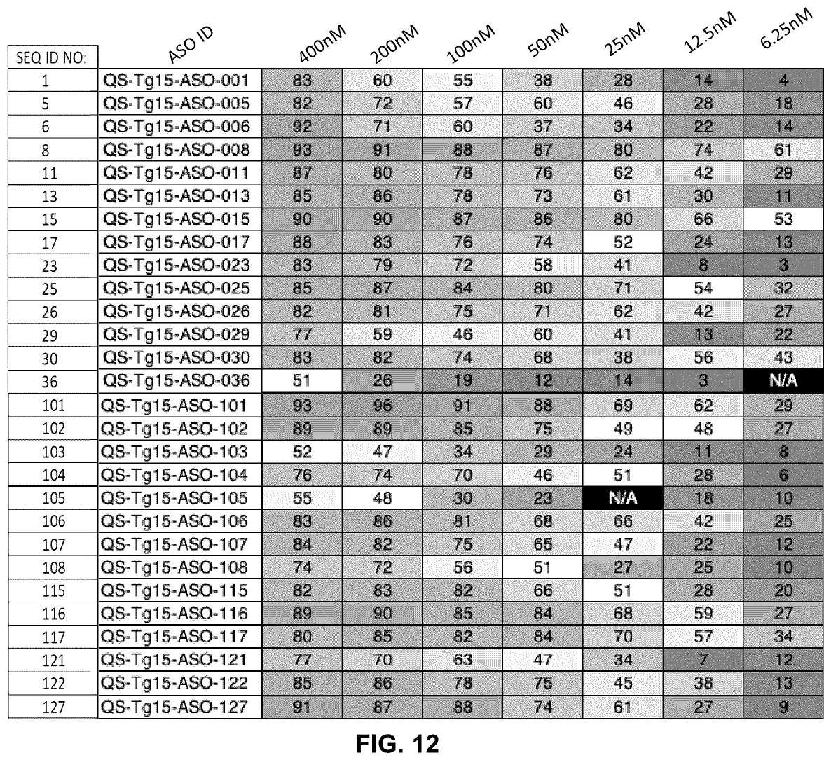

FIG. 12 shows the resulting dose-response data for the lead all-PS backbone candidates targeting UBE3A exons.

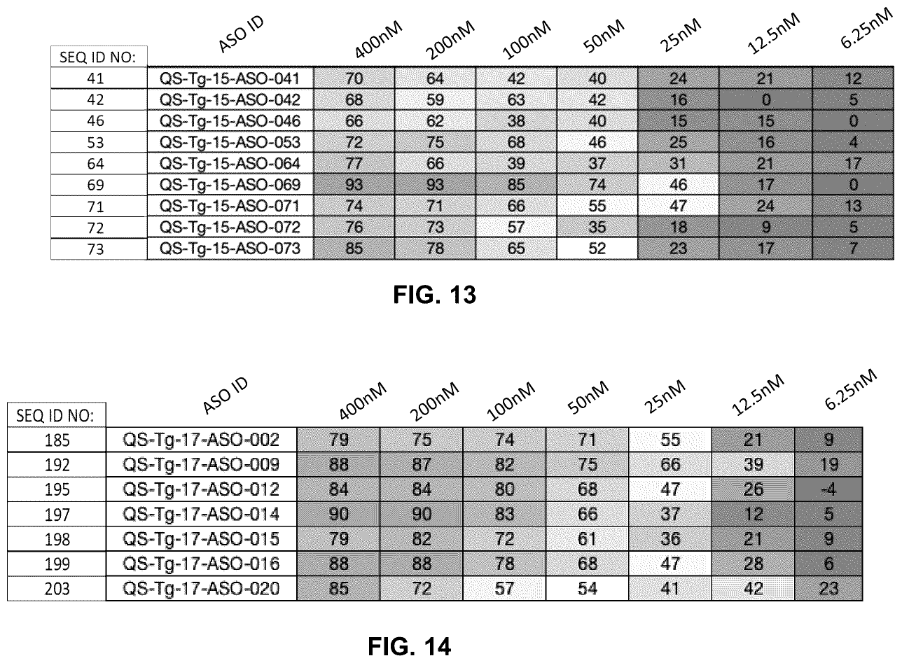

FIG. 13 shows the resulting dose-response data for the lead all-PS backbone candidates targeting UBE3A introns.

FIG. 14 shows the resulting dose-response data for the lead all-PS backbone candidates with 100% mouse homology for rodent in vivo efficacy studies.

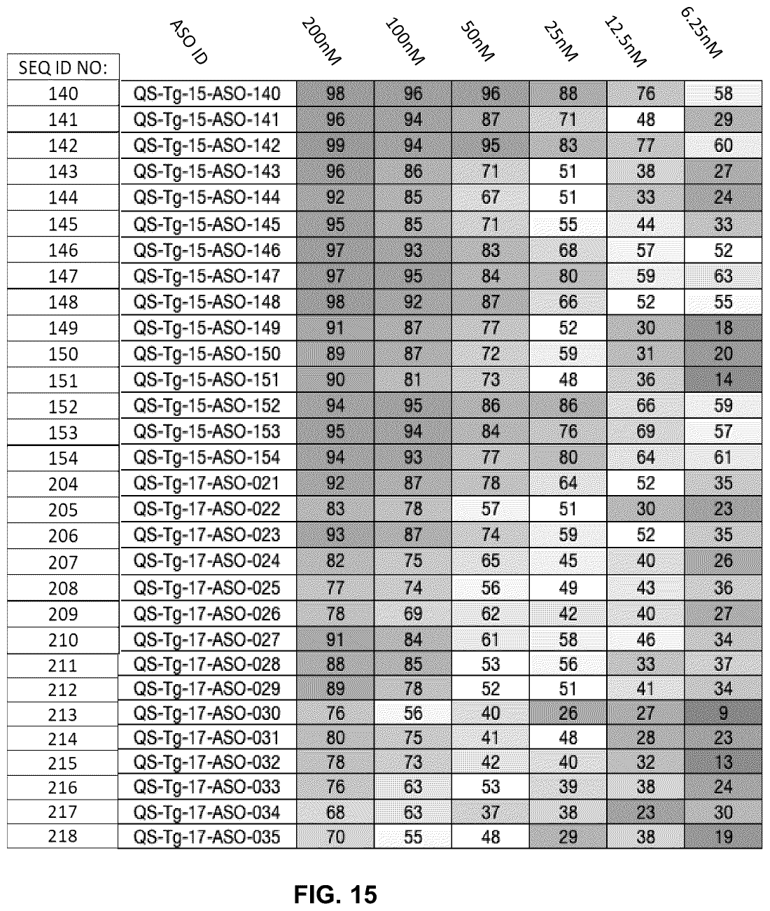

FIG. 15 shows the resulting dose-response data for the PO-modified daughter leads with 100% mouse homology for rodent in vivo efficacy studies.

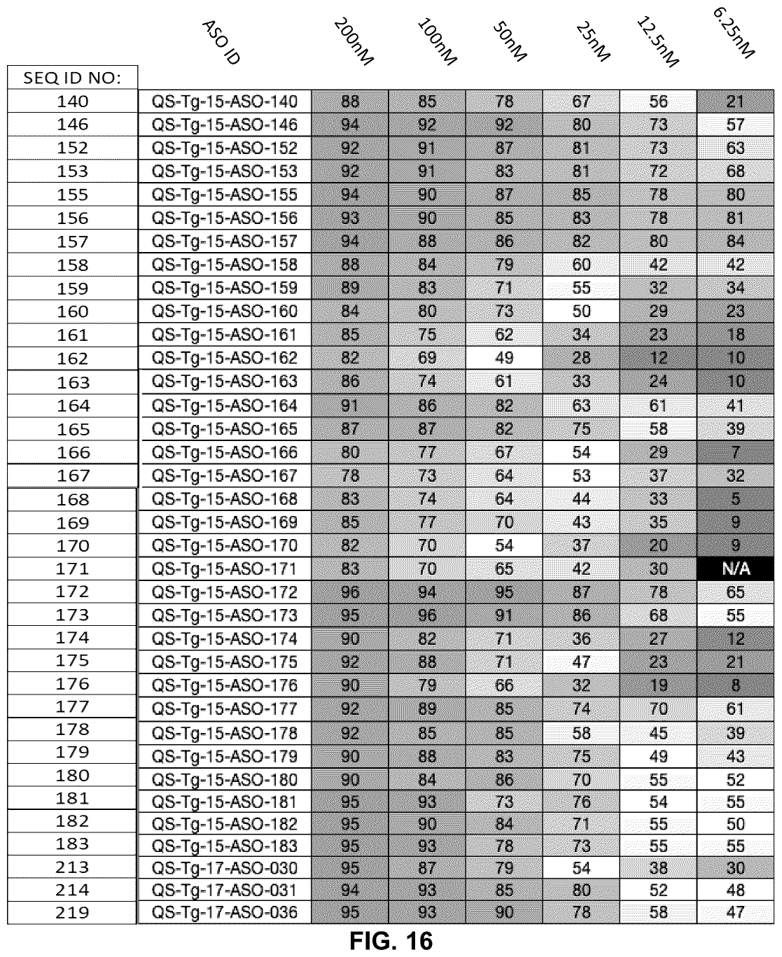

FIG. 16 shows the resulting dose-response data for the PO-modified daughter leads for human clinical candidate studies.

### Example 5—Protein Knockdown of UBE3A Using UBE3A ASOs

ASO-treated Dup15q patient fibroblasts were screened for UBE3A protein knockdown to help determine efficacy and rank ASOs for downstream experiments.

Fibroblasts were plated at 10 k per well of a96-well plate. ASO treatment occurred 48-hours post-plating. To allow for accumulation of protein knockdown, fibroblasts were harvested ˜4.5 days post-ASO treatment for Western Blot analysis.

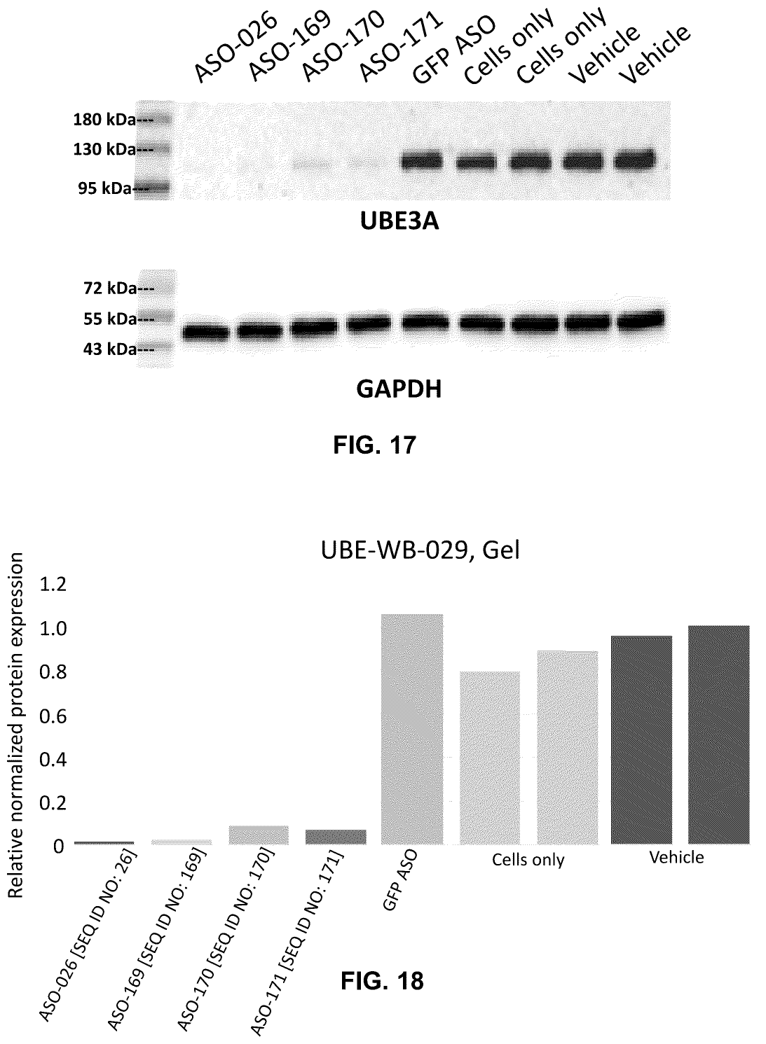

FIG. 17 shows a western blot for a certain candidate lead UBE3A ASO and 3 PO-modified daughter molecules with identical ASO sequences. A GFP-targeting ASO was used as a negative control. UBE3A expression was normalized to the house keeping gene ACTIN and then normalized to a vehicle condition.

FIG. 18 show a quantification of the UBE3A protein knockdown for the abovementioned samples. For the UBE3A blot, exposure was 600s. For GAPDH, exposure was 15s. 5 μg of protein were loaded per lane and a high molecular weight transfer was used. UBE3A Antibody: Rb-E6AP Antibody (Bethyl)—A300-351A (1:1000). Actin Antibody: Msß-Actin—(Cell Signaling)—8H10D10 (1:2000).

### Example 6—Protein Knockdown of UBE3A Using UBE3A-Targeting ASOs

ASO-treated Dup15q patient fibroblasts were screened for UBE3A protein knockdown to help determine efficacy and rank ASOs for downstream experiments.

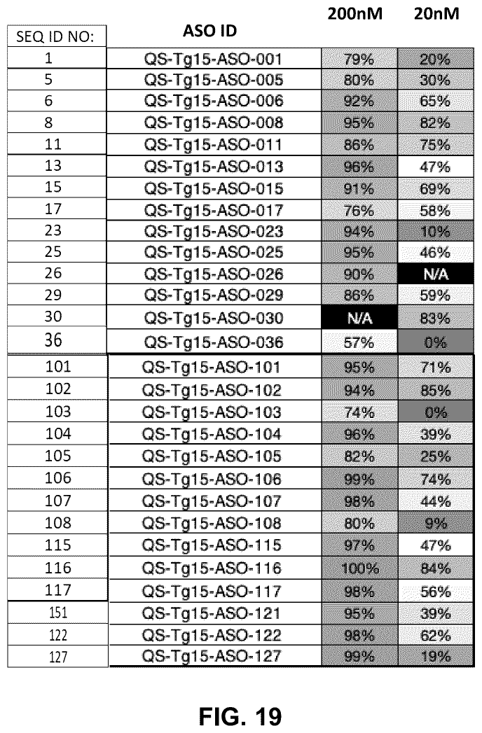

FIGS. 19-22 provide summary tables for UBE3A protein knockdown for candidate leads.

ASO-treated Dup15q patient fibroblasts were screened for UBE3A protein knockdown to help determine efficacy and rank ASOs for downstream experiments. Fibroblasts were plated at 10 k per well of a96-well plate. ASO treatment occurred 48-hours post-plating. To allow for accumulation of protein knockdown, fibroblasts were harvested ˜4.5 days post-ASO treatment for Western Blot analysis. In all experiments, a GFP-targeting ASO was used as a negative control. UBE3A expression was normalized to the house keeping gene ACTIN and then normalized to a vehicle condition. For UBE3A blots, exposure was 600s. For GAPDH, exposure was 15s. 5 μg of protein were loaded per lane and a high molecular weight transfer was used. UBE3A Antibody: Rb-E6AP Antibody (Bethyl)—A 300-351A (1:1000). Actin Antibody: Msß-Actin—(Cell Signaling)—8H10D10 (1:2000).

FIG. 19 provides a table summarizing UBE3A protein knockdown results for lead all-PS backbone candidates targeting UBE3A.

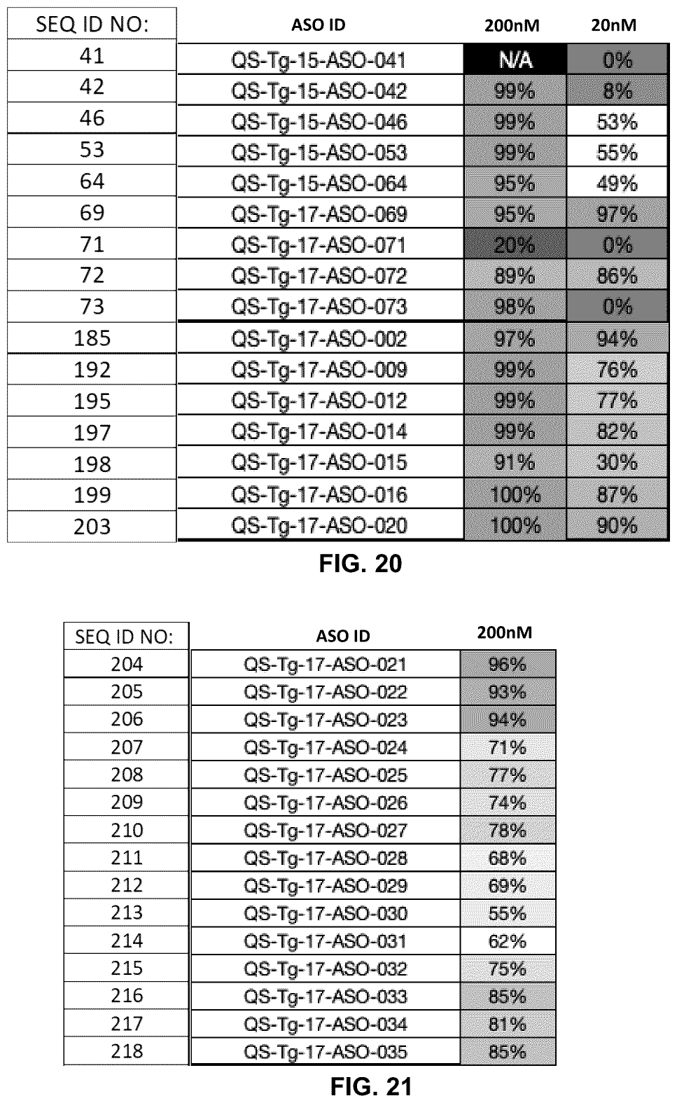

FIG. 20 provides a table summarizing UBE3A protein knockdown results for lead all-PS backbone candidates with 100% mouse homology for rodent in vivo efficacy studies. (C)

FIG. 21 provides a table summarizing UBE3A protein knockdown results for PO-modified daughter leads with 100% mouse homology for rodent in vivo efficacy studies.

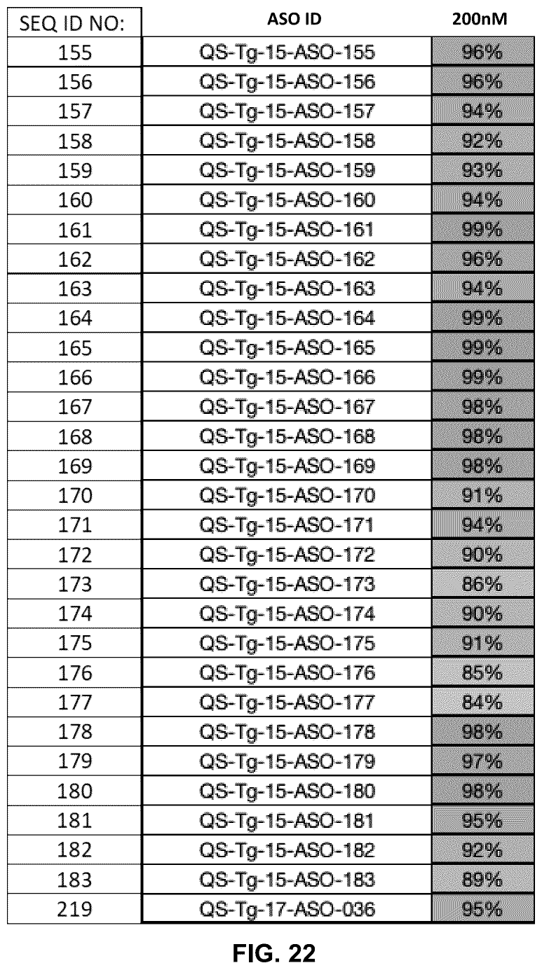

FIG. 22 provides a table summarizing UBE3A protein knockdown results for PO-modified daughter leads for human clinical candidates.

### Example 7—Knockdown of UBE3A Transcript in Human NGN2 Stem Cell-Derived Neurons Using UBE3A Lead Candidates

UBE3A is imprinted in neurons, and this cell type is critical for the pathogenesis of Dup15q. To show that the ASOs of the invention are effective in a disease-relevant human cell type, in this Example, human induced pluripotent stem cell-derived neurons (differentiated via overexpression of the transcription factor NGN2 and small molecule inhibition of SMAD signaling) were treated with UBE3A-targeting ASOs of the invention.

Neurons were plated at a density of 80,000 cells per well on a96-well plate and treated with 100 nM of UBE3A-targeting ASO. ASOs were delivered into the cultured neurons with Endoporter reagent at DIV (day in vitro) 21. Cells were harvested for qPCR 10 days after treatment at DIV31. UBE3A lead candidate ASOs and optimized lead candidate ASOs were screened.

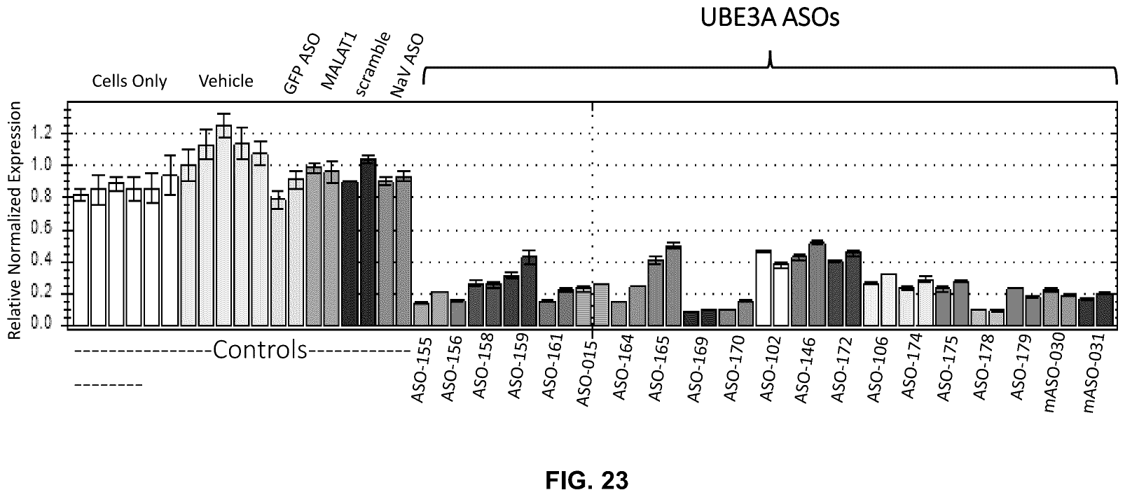

FIG. 23 provides the data summarizing this screening. As shown, many ASOs showed >80% knockdown of UBE3A transcript in human neurons. UBE3A expression levels were normalized to beta tubulin transcript levels (a housekeeping gene used as a reference). All normalized expression was then quantified relative to the first vehicle condition. Each bar represents 3 technical replicates and 1 biological replicate.

### Example 8—Knockdown of UBE3A Transcript in Human Primary Neurons Using UBE3A Lead Candidate ASOs

UBE3A is imprinted in neurons, and that cell type is critical for the pathogenesis of Dup15q. To show that the ASOs of the invention are effective in a relevant human cell type, human primary neurons (derived from a 19-week-old male fetus; acquired from Sciencell) were treated with UBE3A-targeting ASOs. Neurons were plated at a density of 30,000 cells per well on a96-well plate and treated with 500 nM of UBE3A-targeting ASOs. ASOs were delivered gymnotically (no transfection reagent) on DIV 1. Cells were harvested for qPCR 6 days after ASO treatment. A subset of UBE3A lead candidate ASOs and optimized lead candidate ASOs were screened.

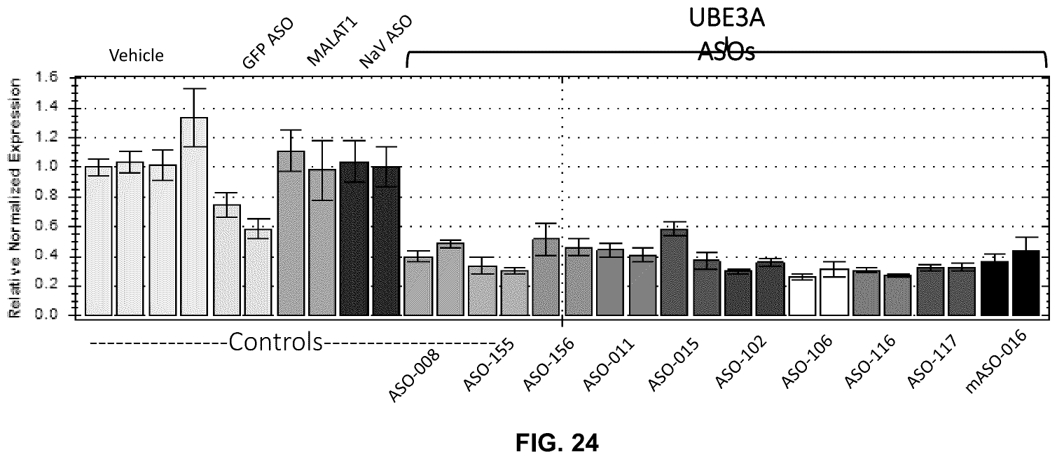

FIG. 24 provides the results summarizing this screen. As shown, many ASOs show >60% knockdown of UBE3A transcript in human primary neurons with gymnotic delivery. UBE3A expression levels were normalized to beta tubulin transcript levels (a housekeeping gene used as a reference). All normalized expression was then quantified relative to the first vehicle condition. Each bar represents 3 technical replicates and 1 biological replicate.

### Example 9—Knockdown of UBE3A Transcript in Non-Human Primate Primary Fibroblast Cultures Using UBE3A Lead Candidate ASOs

UBE3A ASOs that have 100% homology to the corresponding sequence in cynomolgus non-human primates (NHP) were selected for this assay. Lead ASO candidates are screened in vivo in NHP to test for in vivo tolerability, toxicology, PK and PD.

To show that the ASOs of the invention are effective in a relevant NHP cell type, NHP primary fibroblasts (Coriell) were transduced with UBE3A-targeting ASOs. Fibroblasts were plated at a density of 10,000 cells per well on a96-well plate and treated with 200 nM UBE3A ASO. ASOs were transfected into NHP fibroblasts using RNAiMax on DIV 2. Cells were harvested for qPCR 48 hours after ASO treatment. UBE3A lead candidate ASOs and optimized lead candidates were screened.

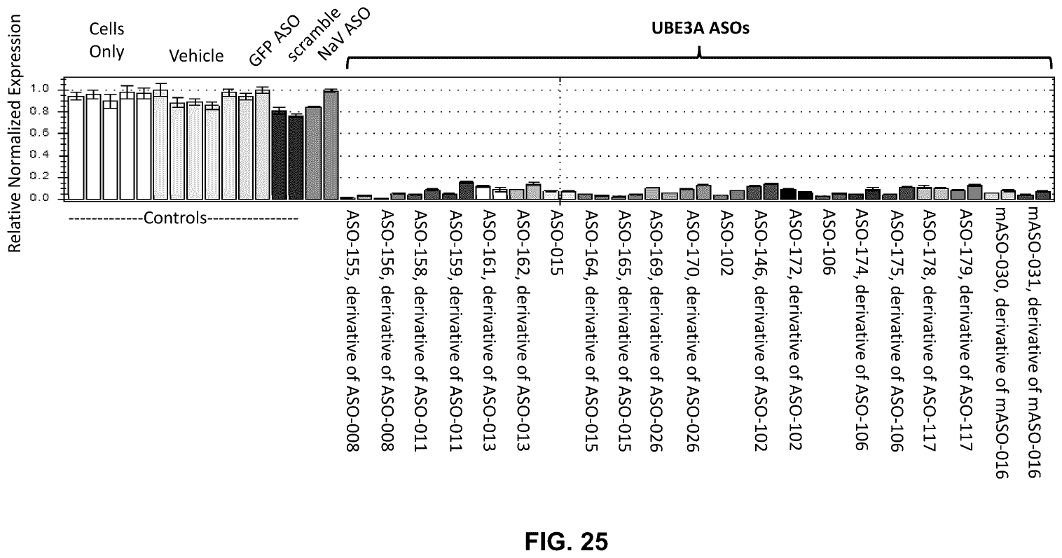

FIG. 25 provides results summarizing this screening. As shown, many ASOs show 80-90% knockdown of UBE3A transcript. UBE3A expression levels were normalized to GAPDH (a housekeeping gene used as a reference). All normalized expression was then quantified relative to the first cells only condition. Each bar represents 2 technical replicates and 1 biological replicate.

### Example 10—Knockdown of UBE3A Transcript in Mouse Primary Cortical Neurons Using Ube3A Lead Candidates

UBE3A is imprinted in neurons, and this cell type is critical for the pathogenesis of Dup15q. Lead ASOs are screened in vivo in mice to test for in vivo tolerability, toxicology, PK and PD.

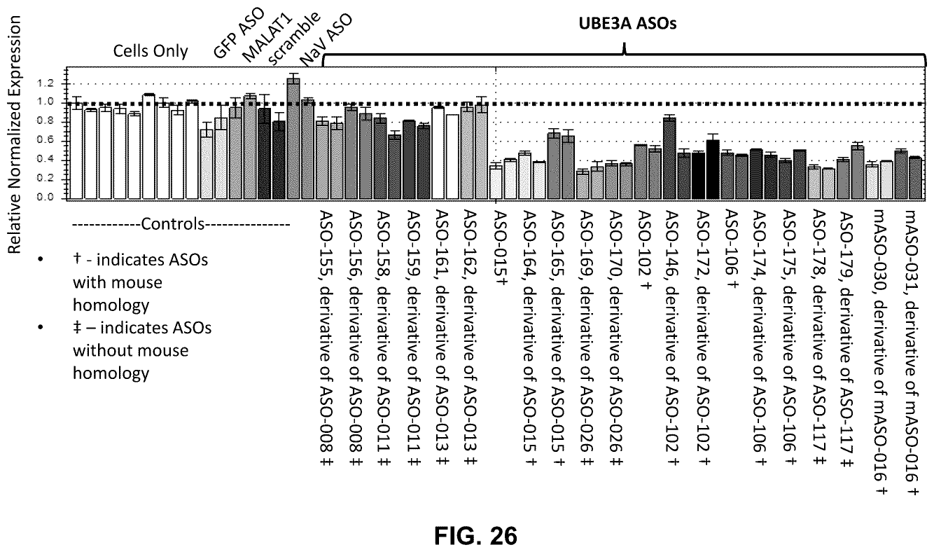

Mouse models of Dup15q are useful for showing proof-of-concept and efficacy in disease model systems in vivo. To show that the ASOs of the invention are effective in a relevant mouse cell type, mouse primary cortical neurons (Brainbits) were treated with UBE3A ASOs. Neurons were plated at 9 k per well on a96-well plate and treated with 1 uM UBE3A ASO. ASOs were delivered gymnotically on DIV 3. Cells were harvested for qPCR 8 days after ASO treatment (DIV11). UBE3A lead candidates and optimized lead candidates were screened. The resulting data from these screens are presented in FIG. 26. As shown, many ASOs show >60% knockdown of UBE3A transcript with gymnotic delivery, especially ASOs with 100% rat homology. UBE3A expression levels were normalized to beta tubulin (used as a housekeeping gene). All normalized expression was then quantified relative to the second cells only condition. Each bar represents 2 technical replicates and 1 biological replicate.

### Example 11—Knockdown of UBE3A Transcript in Rat Primary Cortical Neurons Using Ube3A Lead Candidates

UBE3A is imprinted in neurons, and this cell type is critical for the pathogenesis of Dup15q. Lead ASOs are screened in vivo in rats to test for in vivo tolerability, toxicology, PK and PD.

To show that the ASOs of the invention are effective in a relevant rat cell type, rat primary cortical neurons (Brainbits) were treated with UBE3A ASOs as described herein. Neurons were plated at 9 k per well on a96-well plate and treated with 3 uM UBE3A ASO. ASOs were delivered gymnotically on DIV 3.

Cells were harvested for qPCR 4 days and 8 days after ASO treatment (DIV 7 and DIV11, respectively). UBE3A lead candidates and optimized lead candidates were screened.

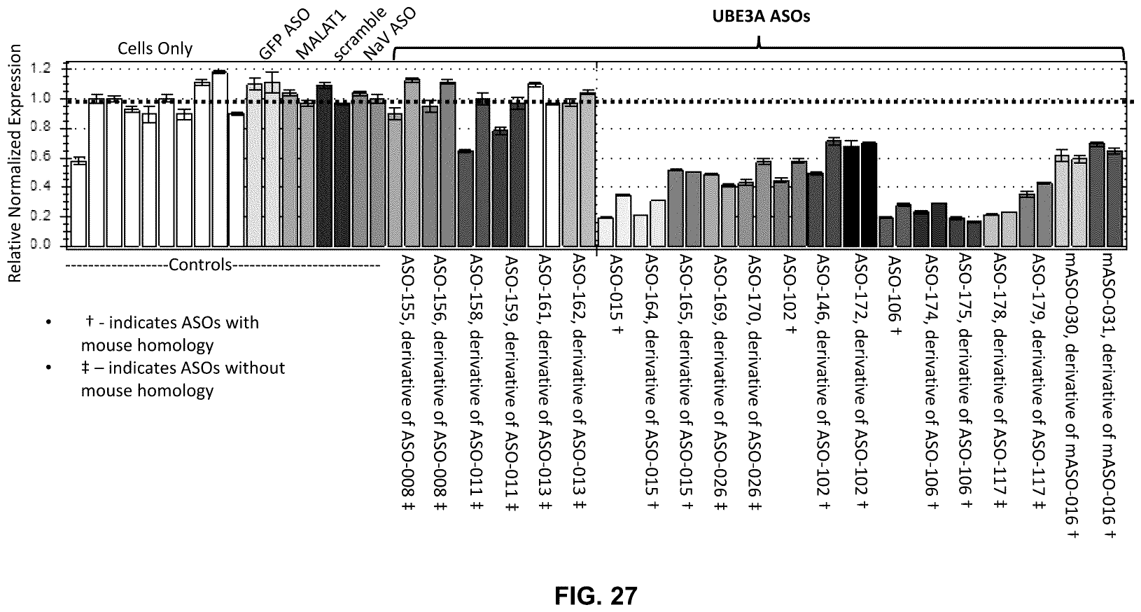

FIG. 27 provides the results summarizing the screens after cells were harvested for qPCR after four days.

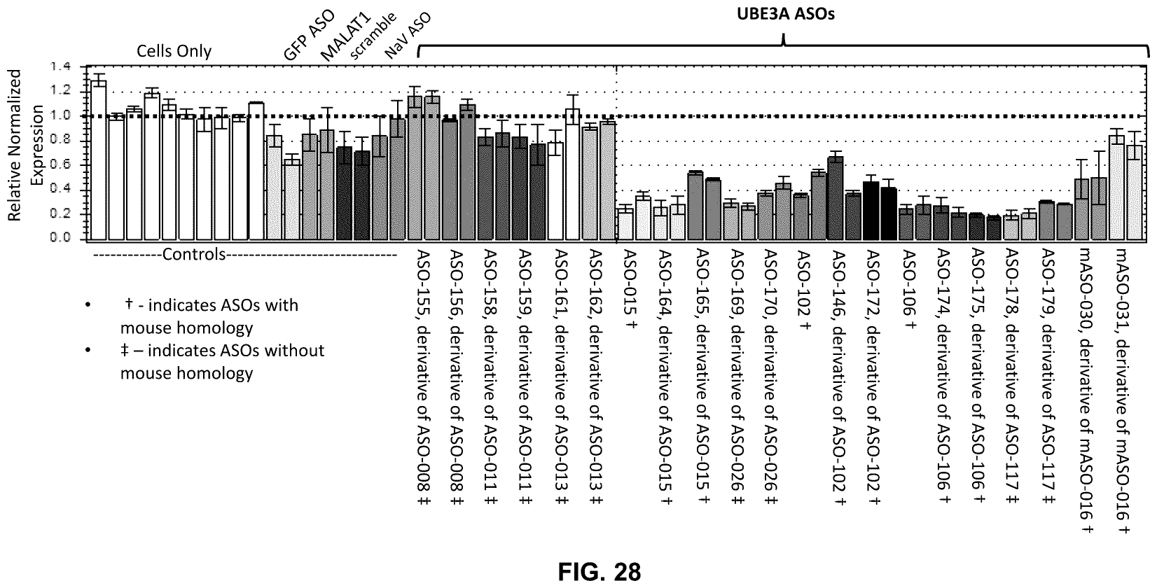

FIG. 28 provides the results summarizing the screens after cells were harvested for qPCR after eight days.

As shown in FIGS. 27-28, many ASOs show >60% knockdown of UBE3A transcript with gymnotic delivery, especially ASOs with 100% rat homology. UBE3A expression levels were normalized to beta tubulin (used as a housekeeping gene). All normalized expression was then quantified relative to the second cells only condition. Each bar represents 2 technical replicates and 1 biological replicate.

What is claimed is:

1. A composition comprising:
a synthetic antisense oligonucleotide (ASO) that inhibits expression of a ubiquitin ligase protein.

2. The composition of claim 1, wherein the protein is ubiquitin protein ligase E3A.

3. The composition of claim 1, wherein the ASO hybridizes to a complementary target in a transcript from the UBE3A gene.

4. The composition of claim 1, wherein a sequence of bases in the ASO has at least 80% identity to one of SEQ ID NOS: 1-228.

5. The composition of claim 1, wherein a sequence of bases in the ASO is at least 90% identical to one of SEQ ID NOS: 1-228, wherein the oligonucleotide can hybridize to, and induce RNaseH-mediated cleavage of, UBE3A pre-mRNA or mRNA.

6. The composition of claim 1, wherein the oligonucleotide comprises two wings flanking a central region of at least 10 DNA bases.

7. The composition of claim 6, wherein at least one wing of the ASO comprises modified RNA bases.

8. The composition of claim 7, wherein each modified RNA base is selected from the group consisting of 2′-O-methoxyethyl RNA and 2′-O-methyl RNA.

9. The composition of claim 1, wherein the ASO comprises at least about 15 bases.

10. The composition of claim 1, wherein the ASO comprises between about 15 about 25 bases.

11. The composition of claim 1, wherein the ASO has a backbone comprising a plurality of phosphorothioate bonds.

12. The composition of claim 1, wherein the ASO has a base sequence that has been screened and determined to not meet a threshold match for any non-target transcripts in humans.

13. The composition of claim 1, wherein the ASO has a base sequence with 0 mismatches to a homologous segment in a non-human primate genome and no more than about 5 mismatches in a homologous segment in a rodent genome.

14. The composition of claim 1, wherein the composition comprises a plurality of ASOs each having a base sequence at least 80% identical to one of SEQ ID NOS: 1-40, 146, 155, 156, 158, 159, 161, 164, 169, 174, 175, 178, 179, 213, and 214 wherein each of the ASOs has a gapmer structure that comprises a central DNA segment flanked by RNA wings.

15. The composition of claim 2, wherein the oligonucleotide has a base sequence with at least a90% match to one of SEQ ID NO: 1-228, with bases linked only by phosphorothioate linkages, the oligonucleotide further comprising a central 12 DNA bases flanked by a 5′ wing and a 3′ wing, the 5′ wing and the 3′ wing each comprising four consecutive 2′ modified RNA bases.

16. The composition of claim 2, wherein the oligonucleotide has a base sequence matching one of SEQ ID NO: 1-40, 146, 155, 156, 158, 159, 161, 164, 169, 174, 175, 178, 179, 213, and 214, with at least a majority of inter-base linkages comprising phosphorothioate linkages, the oligonucleotide further comprising a central 12 DNA bases flanked by a 5′ wing and a 3′ wing, the 5′ wing and the 3′ wing each comprising four consecutive 2′-MOE RNA bases.

17. The composition of claim 1, wherein the ASO is conjugated to a member selected from the group consisting of carbohydrates, cell surface receptor ligands, drug substances, hormones, lipophilic substances, polymers, proteins, peptides, toxins, vitamins, viral proteins, and combinations thereof.

18. A method comprising:
administering to a subject with Dup15q syndrome a composition of any one of claims 1-16 to thereby knock down expression of the UBE3A gene.


SEQUENCE LISTING (228 sequences)

SEQ ID NO: 1 - DNA, 20 bp, synthetic construct
  misc_feature 1..20: note = Synthetic Antisense Oligonucleotide
  source 1..20: mol_type = other DNA
tcatttccacagccctcagt

SEQ ID NO: 2 - DNA, 20 bp, synthetic construct
  misc_feature 1..20: note = Synthetic Antisense Oligonucleotide
  source 1..20: mol_type = other DNA
tcagagcaggagttgttggg

SEQ ID NO: 3 - DNA, 20 bp, synthetic construct
  misc_feature 1..20: note = Synthetic Antisense Oligonucleotide
  source 1..20: mol_type = other DNA
gatttcagttcttccttggt

SEQ ID NO: 4 - DNA, 20 bp, synthetic construct
  misc_feature 1..20: note = Synthetic Antisense Oligonucleotide
  source 1..20: mol_type = other DNA
tccatagcagcagcagaaca

SEQ ID NO: 5 - DNA, 20 bp, synthetic construct
  misc_feature 1..20: note = Synthetic Antisense Oligonucleotide
  source 1..20: mol_type = other DNA
gcttctgagtcttcttccat

SEQ ID NO: 6 - DNA, 20 bp, synthetic construct
  misc_feature 1..20: note = Synthetic Antisense Oligonucleotide
  source 1..20: mol_type = other DNA
gtgagctatcacctatcctt

SEQ ID NO: 7 - DNA, 20 bp, synthetic construct
  misc_feature 1..20: note = Synthetic Antisense Oligonucleotide
  source 1..20: mol_type = other DNA
ttgttgtctccctgtgagct

SEQ ID NO: 8 - DNA, 20 bp, synthetic construct
  misc_feature 1..20: note = Synthetic Antisense Oligonucleotide
  source 1..20: mol_type = other DNA
gcaatctggtgtagaccctt

SEQ ID NO: 9 - DNA, 20 bp, synthetic construct
  misc_feature 1..20: note = Synthetic Antisense Oligonucleotide
  source 1..20: mol_type = other DNA
tcccctcccactacatttgc

SEQ ID NO: 10 - DNA, 20 bp, synthetic construct
  misc_feature 1..20: note = Synthetic Antisense Oligonucleotide
  source 1..20: mol_type = other DNA
tttgtgtccacttcccctcc

SEQ ID NO: 11 - DNA, 20 bp, synthetic construct
  misc_feature 1..20: note = Synthetic Antisense Oligonucleotide
  source 1..20: mol_type = other DNA
gggatgggctcttcatcatc

SEQ ID NO: 12 - DNA, 20 bp, synthetic construct
  misc_feature 1..20: note = Synthetic Antisense Oligonucleotide
  source 1..20: mol_type = other DNA
aggacctttcttgtttcttc

SEQ ID NO: 13 - DNA, 20 bp, synthetic construct
  misc_feature 1..20: note = Synthetic Antisense Oligonucleotide
  source 1..20: mol_type = other DNA
accaagttcagtttccaggg

SEQ ID NO: 14 - DNA, 20 bp, synthetic construct
  misc_feature 1..20: note = Synthetic Antisense Oligonucleotide
  source 1..20: mol_type = other DNA
acctcattcagtggttcatt

SEQ ID NO: 15 - DNA, 20 bp, synthetic construct
  misc_feature 1..20: note = Synthetic Antisense Oligonucleotide
  source 1..20: mol_type = other DNA
ggattcaactgctgtccttg

SEQ ID NO: 16 - DNA, 20 bp, synthetic construct
  misc_feature 1..20: note = Synthetic Antisense Oligonucleotide
  source 1..20: mol_type = other DNA
tcatcaactccttgttctcc

SEQ ID NO: 17 - DNA, 20 bp, synthetic construct
  misc_feature 1..20: note = Synthetic Antisense Oligonucleotide
  source 1..20: mol_type = other DNA
atttcctccacaaccagctg

SEQ ID NO: 18 - DNA, 20 bp, synthetic construct
  misc_feature 1..20: note = Synthetic Antisense Oligonucleotide
  source 1..20: mol_type = other DNA
gccagacccagtactatgcc

SEQ ID NO: 19 - DNA, 20 bp, synthetic construct
  misc_feature 1..20: note = Synthetic Antisense Oligonucleotide
  source 1..20: mol_type = other DNA
ccacattcccttcatactcc

SEQ ID NO: 20 - DNA, 20 bp, synthetic construct
  misc_feature 1..20: note = Synthetic Antisense Oligonucleotide
  source 1..20: mol_type = other DNA
gagtccctggtatagccacc

SEQ ID NO: 21 - DNA, 20 bp, synthetic construct
  misc_feature 1..20: note = Synthetic Antisense Oligonucleotide
  source 1..20: mol_type = other DNA
agtcttttctgttcatctgt

SEQ ID NO: 22 - DNA, 20 bp, synthetic construct
  misc_feature 1..20: note = Synthetic Antisense Oligonucleotide
  source 1..20: mol_type = other DNA
caggtgctctgtctgtgccc

SEQ ID NO: 23 - DNA, 20 bp, synthetic construct
  misc_feature 1..20: note = Synthetic Antisense Oligonucleotide
  source 1..20: mol_type = other DNA
cccacaggtgctctgtctgt

SEQ ID NO: 24 - DNA, 20 bp, synthetic construct
  misc_feature 1..20: note = Synthetic Antisense Oligonucleotide
  source 1..20: mol_type = other DNA
cctagtcctcccacaggtgc

SEQ ID NO: 25 - DNA, 20 bp, synthetic construct
  misc_feature 1..20: note = Synthetic Antisense Oligonucleotide
  source 1..20: mol_type = other DNA
aacctttctgtgtctgggcc

SEQ ID NO: 26 - DNA, 20 bp, synthetic construct
  misc_feature 1..20: note = Synthetic Antisense Oligonucleotide
  source 1..20: mol_type = other DNA
cagcctttttgtactgggac

SEQ ID NO: 27 - DNA, 20 bp, synthetic construct
  misc_feature 1..20: note = Synthetic Antisense Oligonucleotide
  source 1..20: mol_type = other DNA
ttccagcccacatgtcccca

SEQ ID NO: 28 - DNA, 20 bp, synthetic construct
  misc_feature 1..20: note = Synthetic Antisense Oligonucleotide
  source 1..20: mol_type = other DNA
gaaatctgctgttccagccc

SEQ ID NO: 29 - DNA, 20 bp, synthetic construct
  misc_feature 1..20: note = Synthetic Antisense Oligonucleotide
  source 1..20: mol_type = other DNA
aggctcaacctcaagcagta

SEQ ID NO: 30 - DNA, 20 bp, synthetic construct
  misc_feature 1..20: note = Synthetic Antisense Oligonucleotide
  source 1..20: mol_type = other DNA
gggagagtagttctgttggt

SEQ ID NO: 31 - DNA, 20 bp, synthetic construct
  misc_feature 1..20: note = Synthetic Antisense Oligonucleotide
  source 1..20: mol_type = other DNA
cattccaatttctcccttcc

SEQ ID NO: 32 - DNA, 20 bp, synthetic construct
  misc_feature 1..20: note = Synthetic Antisense Oligonucleotide
  source 1..20: mol_type = other DNA
ccctgtcctttcatatacta

SEQ ID NO: 33 - DNA, 20 bp, synthetic construct
  misc_feature 1..20: note = Synthetic Antisense Oligonucleotide
  source 1..20: mol_type = other DNA
ggccaaatgcactttcccca

SEQ ID NO: 34 - DNA, 20 bp, synthetic construct
  misc_feature 1..20: note = Synthetic Antisense Oligonucleotide
  source 1..20: mol_type = other DNA
gcacagtagccatctttttc

SEQ ID NO: 35 - DNA, 20 bp, synthetic construct
  misc_feature 1..20: note = Synthetic Antisense Oligonucleotide
  source 1..20: mol_type = other DNA
tcattcatttccaggtcagc

SEQ ID NO: 36 - DNA, 20 bp, synthetic construct
  misc_feature 1..20: note = Synthetic Antisense Oligonucleotide
  source 1..20: mol_type = other DNA
aggcacaagctcagcacatt

SEQ ID NO: 37 - DNA, 20 bp, synthetic construct
  misc_feature 1..20: note = Synthetic Antisense Oligonucleotide
  source 1..20: mol_type = other DNA
gcattgtcttctttttccac

SEQ ID NO: 38 - DNA, 20 bp, synthetic construct
  misc_feature 1..20: note = Synthetic Antisense Oligonucleotide
  source 1..20: mol_type = other DNA
ccccatgttaccttatcaca

SEQ ID NO: 39 - DNA, 20 bp, synthetic construct
  misc_feature 1..20: note = Synthetic Antisense Oligonucleotide
  source 1..20: mol_type = other DNA
gtccctttcatcaaggtagc

SEQ ID NO: 40 - DNA, 20 bp, synthetic construct
  misc_feature 1..20: note = Synthetic Antisense Oligonucleotide
  source 1..20: mol_type = other DNA
gcacagtggatgagaagcct

SEQ ID NO: 41 - DNA, 20 bp, synthetic construct
  misc_feature 1..20: note = Synthetic Antisense Oligonucleotide
  source 1..20: mol_type = other DNA
gctgctcgcttcctgtacca

SEQ ID NO: 42 - DNA, 20 bp, synthetic construct
  misc_feature 1..20: note = Synthetic Antisense Oligonucleotide
  source 1..20: mol_type = other DNA
cttactgggtgagagtctcc

SEQ ID NO: 43 - DNA, 20 bp, synthetic construct
  misc_feature 1..20: note = Synthetic Antisense Oligonucleotide
  source 1..20: mol_type = other DNA
ttcttacccggcttccacat

SEQ ID NO: 44 - DNA, 20 bp, synthetic construct
  misc_feature 1..20: note = Synthetic Antisense Oligonucleotide
  source 1..20: mol_type = other DNA
tttcttacccggcttccaca

SEQ ID NO: 45 - DNA, 20 bp, synthetic construct
  misc_feature 1..20: note = Synthetic Antisense Oligonucleotide
  source 1..20: mol_type = other DNA
ctttcttacccggcttccac

SEQ ID NO: 46 - DNA, 20 bp, synthetic construct
  misc_feature 1..20: note = Synthetic Antisense Oligonucleotide
  source 1..20: mol_type = other DNA
tacctttctgtgtctgggcc

SEQ ID NO: 47 - DNA, 20 bp, synthetic construct
  misc_feature 1..20: note = Synthetic Antisense Oligonucleotide
  source 1..20: mol_type = other DNA
accttcctgttttcatttgt

SEQ ID NO: 48 - DNA, 20 bp, synthetic construct
  misc_feature 1..20: note = Synthetic Antisense Oligonucleotide
  source 1..20: mol_type = other DNA
acttactgggtgagagtctc

SEQ ID NO: 49 - DNA, 20 bp, synthetic construct
  misc_feature 1..20: note = Synthetic Antisense Oligonucleotide
  source 1..20: mol_type = other DNA
taccttcctgttttcatttg

SEQ ID NO: 50 - DNA, 20 bp, synthetic construct
  misc_feature 1..20: note = Synthetic Antisense Oligonucleotide
  source 1..20: mol_type = other DNA
aacttactgggtgagagtct

SEQ ID NO: 51 - DNA, 20 bp, synthetic construct
  misc_feature 1..20: note = Synthetic Antisense Oligonucleotide
  source 1..20: mol_type = other DNA
gccctcccttcccatcaatc

SEQ ID NO: 52 - DNA, 20 bp, synthetic construct
  misc_feature 1..20: note = Synthetic Antisense Oligonucleotide
  source 1..20: mol_type = other DNA
tccccacacctctgactagt

SEQ ID NO: 53 - DNA, 20 bp, synthetic construct
  misc_feature 1..20: note = Synthetic Antisense Oligonucleotide
  source 1..20: mol_type = other DNA
gggtggtgggctgggaccaa

SEQ ID NO: 54 - DNA, 20 bp, synthetic construct
  misc_feature 1..20: note = Synthetic Antisense Oligonucleotide
  source 1..20: mol_type = other DNA
actgacccctagttctgcct

SEQ ID NO: 55 - DNA, 20 bp, synthetic construct
  misc_feature 1..20: note = Synthetic Antisense Oligonucleotide
  source 1..20: mol_type = other DNA
ccttggctctcccctccctt

SEQ ID NO: 56 - DNA, 20 bp, synthetic construct
  misc_feature 1..20: note = Synthetic Antisense Oligonucleotide
  source 1..20: mol_type = other DNA
ggacccatggcctttgagct

SEQ ID NO: 57 - DNA, 20 bp, synthetic construct
  misc_feature 1..20: note = Synthetic Antisense Oligonucleotide
  source 1..20: mol_type = other DNA
tgacaccatacctcccctct

SEQ ID NO: 58 - DNA, 20 bp, synthetic construct
  misc_feature 1..20: note = Synthetic Antisense Oligonucleotide
  source 1..20: mol_type = other DNA
cccagcactactgcccacta

SEQ ID NO: 59 - DNA, 20 bp, synthetic construct
  misc_feature 1..20: note = Synthetic Antisense Oligonucleotide
  source 1..20: mol_type = other DNA
accccagccatcccagcact

SEQ ID NO: 60 - DNA, 20 bp, synthetic construct
  misc_feature 1..20: note = Synthetic Antisense Oligonucleotide
  source 1..20: mol_type = other DNA
gagtctctctctttcccagt

SEQ ID NO: 61 - DNA, 20 bp, synthetic construct
  misc_feature 1..20: note = Synthetic Antisense Oligonucleotide
  source 1..20: mol_type = other DNA
cctctgacccttgagtctcc

SEQ ID NO: 62 - DNA, 20 bp, synthetic construct
  misc_feature 1..20: note = Synthetic Antisense Oligonucleotide
  source 1..20: mol_type = other DNA
caccctacctgggtccctca

SEQ ID NO: 63 - DNA, 20 bp, synthetic construct
  misc_feature 1..20: note = Synthetic Antisense Oligonucleotide
  source 1..20: mol_type = other DNA
cctctcttccagtcccctct

SEQ ID NO: 64 - DNA, 20 bp, synthetic construct
  misc_feature 1..20: note = Synthetic Antisense Oligonucleotide
  source 1..20: mol_type = other DNA
ggtcaactctcaggcccact

SEQ ID NO: 65 - DNA, 20 bp, synthetic construct
  misc_feature 1..20: note = Synthetic Antisense Oligonucleotide
  source 1..20: mol_type = other DNA
ggtgcagcttctccatcctg

SEQ ID NO: 66 - DNA, 20 bp, synthetic construct
  misc_feature 1..20: note = Synthetic Antisense Oligonucleotide
  source 1..20: mol_type = other DNA
ccctccagcatcagatgtca

SEQ ID NO: 67 - DNA, 20 bp, synthetic construct
  misc_feature 1..20: note = Synthetic Antisense Oligonucleotide
  source 1..20: mol_type = other DNA
gacacacctggtctccacca

SEQ ID NO: 68 - DNA, 20 bp, synthetic construct
  misc_feature 1..20: note = Synthetic Antisense Oligonucleotide
  source 1..20: mol_type = other DNA
cttcacccattcccctcagt

SEQ ID NO: 69 - DNA, 20 bp, synthetic construct
  misc_feature 1..20: note = Synthetic Antisense Oligonucleotide
  source 1..20: mol_type = other DNA
tgggctcctgtgtctgtcag

SEQ ID NO: 70 - DNA, 20 bp, synthetic construct
  misc_feature 1..20: note = Synthetic Antisense Oligonucleotide
  source 1..20: mol_type = other DNA
gccctccagtgaccctgcca

SEQ ID NO: 71 - DNA, 20 bp, synthetic construct
  misc_feature 1..20: note = Synthetic Antisense Oligonucleotide
  source 1..20: mol_type = other DNA
gtccaggagtctttcagctt

SEQ ID NO: 72 - DNA, 20 bp, synthetic construct
  misc_feature 1..20: note = Synthetic Antisense Oligonucleotide
  source 1..20: mol_type = other DNA
ctgcattccactgtgccagc

SEQ ID NO: 73 - DNA, 20 bp, synthetic construct
  misc_feature 1..20: note = Synthetic Antisense Oligonucleotide
  source 1..20: mol_type = other DNA
gggtcttcctagtttgttcc

SEQ ID NO: 74 - DNA, 20 bp, synthetic construct
  misc_feature 1..20: note = Synthetic Antisense Oligonucleotide
  source 1..20: mol_type = other DNA
gtttccttatgccagttccc

SEQ ID NO: 75 - DNA, 20 bp, synthetic construct
  misc_feature 1..20: note = Synthetic Antisense Oligonucleotide
  source 1..20: mol_type = other DNA
atgagcagggtccagcagga

SEQ ID NO: 76 - DNA, 20 bp, synthetic construct
  misc_feature 1..20: note = Synthetic Antisense Oligonucleotide
  source 1..20: mol_type = other DNA
ttgccacttcccttccctgc

SEQ ID NO: 77 - DNA, 20 bp, synthetic construct
  misc_feature 1..20: note = Synthetic Antisense Oligonucleotide
  source 1..20: mol_type = other DNA
gactctacactgtccagcca

SEQ ID NO: 78 - DNA, 20 bp, synthetic construct
  misc_feature 1..20: note = Synthetic Antisense Oligonucleotide
  source 1..20: mol_type = other DNA
ctccattagctcctcagagt

SEQ ID NO: 79 - DNA, 20 bp, synthetic construct
  misc_feature 1..20: note = Synthetic Antisense Oligonucleotide
  source 1..20: mol_type = other DNA
tcctcctaacctcttccaga

SEQ ID NO: 80 - DNA, 20 bp, synthetic construct
  misc_feature 1..20: note = Synthetic Antisense Oligonucleotide
  source 1..20: mol_type = other DNA
ccacatctcagccattcctt

SEQ ID NO: 81 - DNA, 18 bp, synthetic construct
  misc_feature 1..18: note = Synthetic Antisense Oligonucleotide
  source 1..18: mol_type = other DNA
gctatcacctatccttga

SEQ ID NO: 82 - DNA, 18 bp, synthetic construct
  misc_feature 1..18: note = Synthetic Antisense Oligonucleotide
  source 1..18: mol_type = other DNA
gtctccctgtgagctatc

SEQ ID NO: 83 - DNA, 18 bp, synthetic construct
  misc_feature 1..18: note = Synthetic Antisense Oligonucleotide
  source 1..18: mol_type = other DNA
tctggtgtagacccttct

SEQ ID NO: 84 - DNA, 18 bp, synthetic construct
  misc_feature 1..18: note = Synthetic Antisense Oligonucleotide
  source 1..18: mol_type = other DNA
cctcccactacatttgca

SEQ ID NO: 85 - DNA, 18 bp, synthetic construct
  misc_feature 1..18: note = Synthetic Antisense Oligonucleotide
  source 1..18: mol_type = other DNA
attcaactgctgtccttg

SEQ ID NO: 86 - DNA, 18 bp, synthetic construct
  misc_feature 1..18: note = Synthetic Antisense Oligonucleotide
  source 1..18: mol_type = other DNA
tgcaggattttccatagc

SEQ ID NO: 87 - DNA, 18 bp, synthetic construct
  misc_feature 1..18: note = Synthetic Antisense Oligonucleotide
  source 1..18: mol_type = other DNA
tagccagacccagtacta

SEQ ID NO: 88 - DNA, 18 bp, synthetic construct
  misc_feature 1..18: note = Synthetic Antisense Oligonucleotide
  source 1..18: mol_type = other DNA
gtgagagtctcccaagtc

SEQ ID NO: 89 - DNA, 18 bp, synthetic construct
  misc_feature 1..18: note = Synthetic Antisense Oligonucleotide
  source 1..18: mol_type = other DNA
cacattcccttcatactc

SEQ ID NO: 90 - DNA, 18 bp, synthetic construct
  misc_feature 1..18: note = Synthetic Antisense Oligonucleotide
  source 1..18: mol_type = other DNA
ggcttccacatataagca

SEQ ID NO: 91 - DNA, 18 bp, synthetic construct
  misc_feature 1..18: note = Synthetic Antisense Oligonucleotide
  source 1..18: mol_type = other DNA
atctgctgttccagccca

SEQ ID NO: 92 - DNA, 18 bp, synthetic construct
  misc_feature 1..18: note = Synthetic Antisense Oligonucleotide
  source 1..18: mol_type = other DNA
gagagtagttctgttggt

SEQ ID NO: 93 - DNA, 18 bp, synthetic construct
  misc_feature 1..18: note = Synthetic Antisense Oligonucleotide
  source 1..18: mol_type = other DNA
acatactgtggcatgagt

SEQ ID NO: 94 - DNA, 18 bp, synthetic construct
  misc_feature 1..18: note = Synthetic Antisense Oligonucleotide
  source 1..18: mol_type = other DNA
gcactttccccagtaaac

SEQ ID NO: 95 - DNA, 18 bp, synthetic construct
  misc_feature 1..18: note = Synthetic Antisense Oligonucleotide
  source 1..18: mol_type = other DNA
gcaataggcttgactacc

SEQ ID NO: 96 - DNA, 18 bp, synthetic construct
  misc_feature 1..18: note = Synthetic Antisense Oligonucleotide
  source 1..18: mol_type = other DNA
gggagactttggattgtc

SEQ ID NO: 97 - DNA, 18 bp, synthetic construct
  misc_feature 1..18: note = Synthetic Antisense Oligonucleotide
  source 1..18: mol_type = other DNA
ccaggtcagcttactgta

SEQ ID NO: 98 - DNA, 18 bp, synthetic construct
  misc_feature 1..18: note = Synthetic Antisense Oligonucleotide
  source 1..18: mol_type = other DNA
gctcagcacattagctat

SEQ ID NO: 99 - DNA, 18 bp, synthetic construct
  misc_feature 1..18: note = Synthetic Antisense Oligonucleotide
  source 1..18: mol_type = other DNA
ccccatgttaccttatca

SEQ ID NO: 100 - DNA, 18 bp, synthetic construct
  misc_feature 1..18: note = Synthetic Antisense Oligonucleotide
  source 1..18: mol_type = other DNA
ggtccctttcatcaaggt

SEQ ID NO: 101 - DNA, 20 bp, synthetic construct
  misc_feature 1..20: note = Synthetic Antisense Oligonucleotide
  source 1..20: mol_type = other DNA
ggagggatgaggatcacaga

SEQ ID NO: 102 - DNA, 20 bp, synthetic construct
  misc_feature 1..20: note = Synthetic Antisense Oligonucleotide
  source 1..20: mol_type = other DNA
gcttgctcctttcttggagg

SEQ ID NO: 103 - DNA, 20 bp, synthetic construct
  misc_feature 1..20: note = Synthetic Antisense Oligonucleotide
  source 1..20: mol_type = other DNA
tatctcagagcaggagttgt

SEQ ID NO: 104 - DNA, 20 bp, synthetic construct
  misc_feature 1..20: note = Synthetic Antisense Oligonucleotide
  source 1..20: mol_type = other DNA
gctctgtaccaatgcctcag

SEQ ID NO: 105 - DNA, 20 bp, synthetic construct
  misc_feature 1..20: note = Synthetic Antisense Oligonucleotide
  source 1..20: mol_type = other DNA
cagaacatgcagctttttcc

SEQ ID NO: 106 - DNA, 20 bp, synthetic construct
  misc_feature 1..20: note = Synthetic Antisense Oligonucleotide
  source 1..20: mol_type = other DNA
gccatttccagatattcagg

SEQ ID NO: 107 - DNA, 20 bp, synthetic construct
  misc_feature 1..20: note = Synthetic Antisense Oligonucleotide
  source 1..20: mol_type = other DNA
tcagttttccttgggctgca

SEQ ID NO: 108 - DNA, 20 bp, synthetic construct
  misc_feature 1..20: note = Synthetic Antisense Oligonucleotide
  source 1..20: mol_type = other DNA
gttgctgaaatgtctccatc

SEQ ID NO: 109 - DNA, 20 bp, synthetic construct
  misc_feature 1..20: note = Synthetic Antisense Oligonucleotide
  source 1..20: mol_type = other DNA
ccctcccactacatttgcat

SEQ ID NO: 110 - DNA, 20 bp, synthetic construct
  misc_feature 1..20: note = Synthetic Antisense Oligonucleotide
  source 1..20: mol_type = other DNA
ctagaacctcattcagtggt

SEQ ID NO: 111 - DNA, 20 bp, synthetic construct
  misc_feature 1..20: note = Synthetic Antisense Oligonucleotide
  source 1..20: mol_type = other DNA
gattcaactgctgtccttga

SEQ ID NO: 112 - DNA, 20 bp, synthetic construct
  misc_feature 1..20: note = Synthetic Antisense Oligonucleotide
  source 1..20: mol_type = other DNA
ccacatacaactgcttcttc

SEQ ID NO: 113 - DNA, 20 bp, synthetic construct
  misc_feature 1..20: note = Synthetic Antisense Oligonucleotide
  source 1..20: mol_type = other DNA
ccagacccagtactatgcca

SEQ ID NO: 114 - DNA, 20 bp, synthetic construct
  misc_feature 1..20: note = Synthetic Antisense Oligonucleotide
  source 1..20: mol_type = other DNA
ttcccagaactccctaatca

SEQ ID NO: 115 - DNA, 20 bp, synthetic construct
  misc_feature 1..20: note = Synthetic Antisense Oligonucleotide
  source 1..20: mol_type = other DNA
ggtaacctttctgtgtctgg

SEQ ID NO: 116 - DNA, 20 bp, synthetic construct
  misc_feature 1..20: note = Synthetic Antisense Oligonucleotide
  source 1..20: mol_type = other DNA
ggccttcaacaatctctctt

SEQ ID NO: 117 - DNA, 20 bp, synthetic construct
  misc_feature 1..20: note = Synthetic Antisense Oligonucleotide
  source 1..20: mol_type = other DNA
gcctttttgtactgggacac

SEQ ID NO: 118 - DNA, 20 bp, synthetic construct
  misc_feature 1..20: note = Synthetic Antisense Oligonucleotide
  source 1..20: mol_type = other DNA
tctgctgttccagcccacat

SEQ ID NO: 119 - DNA, 20 bp, synthetic construct
  misc_feature 1..20: note = Synthetic Antisense Oligonucleotide
  source 1..20: mol_type = other DNA
atctgctgttccagcccaca

SEQ ID NO: 120 - DNA, 20 bp, synthetic construct
  misc_feature 1..20: note = Synthetic Antisense Oligonucleotide
  source 1..20: mol_type = other DNA
ctaaagttctgagggctgca

SEQ ID NO: 121 - DNA, 20 bp, synthetic construct
  misc_feature 1..20: note = Synthetic Antisense Oligonucleotide
  source 1..20: mol_type = other DNA
catactgtggcatgagttgt

SEQ ID NO: 122 - DNA, 20 bp, synthetic construct
  misc_feature 1..20: note = Synthetic Antisense Oligonucleotide
  source 1..20: mol_type = other DNA
gactaccatttcatttggcc

SEQ ID NO: 123 - DNA, 20 bp, synthetic construct
  misc_feature 1..20: note = Synthetic Antisense Oligonucleotide
  source 1..20: mol_type = other DNA
catttccaggtcagcttact

SEQ ID NO: 124 - DNA, 20 bp, synthetic construct
  misc_feature 1..20: note = Synthetic Antisense Oligonucleotide
  source 1..20: mol_type = other DNA
caccaaggcacaagctcagc

SEQ ID NO: 125 - DNA, 20 bp, synthetic construct
  misc_feature 1..20: note = Synthetic Antisense Oligonucleotide
  source 1..20: mol_type = other DNA
aaagctgcatttttcctgcc

SEQ ID NO: 126 - DNA, 20 bp, synthetic construct
  misc_feature 1..20: note = Synthetic Antisense Oligonucleotide
  source 1..20: mol_type = other DNA
acagtgttctaaaggctggc

SEQ ID NO: 127 - DNA, 20 bp, synthetic construct
  misc_feature 1..20: note = Synthetic Antisense Oligonucleotide
  source 1..20: mol_type = other DNA
cagacacatcatcagggcct

SEQ ID NO: 128 - DNA, 20 bp, synthetic construct
  misc_feature 1..20: note = Synthetic Antisense Oligonucleotide
  source 1..20: mol_type = other DNA
acagacacatcatcagggcc

SEQ ID NO: 129 - DNA, 20 bp, synthetic construct
  misc_feature 1..20: note = Synthetic Antisense Oligonucleotide
  source 1..20: mol_type = other DNA
cacagacacatcatcagggc

SEQ ID NO: 130 - DNA, 20 bp, synthetic construct
  misc_feature 1..20: note = Synthetic Antisense Oligonucleotide
  source 1..20: mol_type = other DNA
gactcagggatgggctcttc

SEQ ID NO: 131 - DNA, 20 bp, synthetic construct
  misc_feature 1..20: note = Synthetic Antisense Oligonucleotide
  source 1..20: mol_type = other DNA
ggactcagggatgggctctt

SEQ ID NO: 132 - DNA, 20 bp, synthetic construct
  misc_feature 1..20: note = Synthetic Antisense Oligonucleotide
  source 1..20: mol_type = other DNA
tggactcagggatgggctct

SEQ ID NO: 133 - DNA, 20 bp, synthetic construct
  misc_feature 1..20: note = Synthetic Antisense Oligonucleotide
  source 1..20: mol_type = other DNA
tcccttccttccatctttct

SEQ ID NO: 134 - DNA, 20 bp, synthetic construct
  misc_feature 1..20: note = Synthetic Antisense Oligonucleotide
  source 1..20: mol_type = other DNA
ctcccttccttccatctttc

SEQ ID NO: 135 - DNA, 20 bp, synthetic construct
  misc_feature 1..20: note = Synthetic Antisense Oligonucleotide
  source 1..20: mol_type = other DNA
acatactgtggcatgagttg

SEQ ID NO: 136 - DNA, 20 bp, synthetic construct
  misc_feature 1..20: note = Synthetic Antisense Oligonucleotide
  source 1..20: mol_type = other DNA
caatcagagtaaactgaccc

SEQ ID NO: 137 - DNA, 20 bp, synthetic construct
  misc_feature 1..20: note = Synthetic Antisense Oligonucleotide
  source 1..20: mol_type = other DNA
gacaggaagcacaaaactca

SEQ ID NO: 138 - DNA, 20 bp, synthetic construct
  misc_feature 1..20: note = Synthetic Antisense Oligonucleotide
  source 1..20: mol_type = other DNA
ggacaagtgcatcatctatg

SEQ ID NO: 139 - DNA, 20 bp, synthetic construct
  misc_feature 1..20: note = Synthetic Antisense Oligonucleotide
  source 1..20: mol_type = other DNA
taaatagccagacccagtac

SEQ ID NO: 140 - DNA, 20 bp, synthetic construct
  misc_feature 1..20: note = Synthetic Antisense Oligonucleotide
  source 1..20: mol_type = other DNA
ggattcaactgctgtccttg

SEQ ID NO: 141 - DNA, 20 bp, synthetic construct
  misc_feature 1..20: note = Synthetic Antisense Oligonucleotide
  source 1..20: mol_type = other DNA
ggattcaactgctgtccttg

SEQ ID NO: 142 - DNA, 20 bp, synthetic construct
  misc_feature 1..20: note = Synthetic Antisense Oligonucleotide
  source 1..20: mol_type = other DNA
ggattcaactgctgtccttg

SEQ ID NO: 143 - DNA, 20 bp, synthetic construct
  misc_feature 1..20: note = Synthetic Antisense Oligonucleotide
  source 1..20: mol_type = other DNA
aacctttctgtgtctgggcc

SEQ ID NO: 144 - DNA, 20 bp, synthetic construct
  misc_feature 1..20: note = Synthetic Antisense Oligonucleotide
  source 1..20: mol_type = other DNA
aacctttctgtgtctgggcc

SEQ ID NO: 145 - DNA, 20 bp, synthetic construct
  misc_feature 1..20: note = Synthetic Antisense Oligonucleotide
  source 1..20: mol_type = other DNA
aacctttctgtgtctgggcc

SEQ ID NO: 146 - DNA, 20 bp, synthetic construct
  misc_feature 1..20: note = Synthetic Antisense Oligonucleotide
  source 1..20: mol_type = other DNA
gcttgctcctttcttggagg

SEQ ID NO: 147 - DNA, 20 bp, synthetic construct
  misc_feature 1..20: note = Synthetic Antisense Oligonucleotide
  source 1..20: mol_type = other DNA
gcttgctcctttcttggagg

SEQ ID NO: 148 - DNA, 20 bp, synthetic construct
  misc_feature 1..20: note = Synthetic Antisense Oligonucleotide
  source 1..20: mol_type = other DNA
gcttgctcctttcttggagg

SEQ ID NO: 149 - DNA, 20 bp, synthetic construct
  misc_feature 1..20: note = Synthetic Antisense Oligonucleotide
  source 1..20: mol_type = other DNA
ggtaacctttctgtgtctgg

SEQ ID NO: 150 - DNA, 20 bp, synthetic construct
  misc_feature 1..20: note = Synthetic Antisense Oligonucleotide
  source 1..20: mol_type = other DNA
ggtaacctttctgtgtctgg

SEQ ID NO: 151 - DNA, 20 bp, synthetic construct
  misc_feature 1..20: note = Synthetic Antisense Oligonucleotide
  source 1..20: mol_type = other DNA
ggtaacctttctgtgtctgg

SEQ ID NO: 152 - DNA, 20 bp, synthetic construct
  misc_feature 1..20: note = Synthetic Antisense Oligonucleotide
  source 1..20: mol_type = other DNA
ggccttcaacaatctctctt

SEQ ID NO: 153 - DNA, 20 bp, synthetic construct
  misc_feature 1..20: note = Synthetic Antisense Oligonucleotide
  source 1..20: mol_type = other DNA
ggccttcaacaatctctctt

SEQ ID NO: 154 - DNA, 20 bp, synthetic construct
  misc_feature 1..20: note = Synthetic Antisense Oligonucleotide
  source 1..20: mol_type = other DNA
ggccttcaacaatctctctt

SEQ ID NO: 155 - DNA, 20 bp, synthetic construct
  misc_feature 1..20: note = Synthetic Antisense Oligonucleotide
  source 1..20: mol_type = other DNA
gcaatctggtgtagaccctt

SEQ ID NO: 156 - DNA, 20 bp, synthetic construct
  misc_feature 1..20: note = Synthetic Antisense Oligonucleotide
  source 1..20: mol_type = other DNA
gcaatctggtgtagaccctt

SEQ ID NO: 157 - DNA, 20 bp, synthetic construct
  misc_feature 1..20: note = Synthetic Antisense Oligonucleotide
  source 1..20: mol_type = other DNA
gcaatctggtgtagaccctt

SEQ ID NO: 158 - DNA, 20 bp, synthetic construct
  misc_feature 1..20: note = Synthetic Antisense Oligonucleotide
  source 1..20: mol_type = other DNA
gggatgggctcttcatcatc

SEQ ID NO: 159 - DNA, 20 bp, synthetic construct
  misc_feature 1..20: note = Synthetic Antisense Oligonucleotide
  source 1..20: mol_type = other DNA
gggatgggctcttcatcatc

SEQ ID NO: 160 - DNA, 20 bp, synthetic construct
  misc_feature 1..20: note = Synthetic Antisense Oligonucleotide
  source 1..20: mol_type = other DNA
gggatgggctcttcatcatc

SEQ ID NO: 161 - DNA, 20 bp, synthetic construct
  misc_feature 1..20: note = Synthetic Antisense Oligonucleotide
  source 1..20: mol_type = other DNA
accaagttcagtttccaggg

SEQ ID NO: 162 - DNA, 20 bp, synthetic construct
  misc_feature 1..20: note = Synthetic Antisense Oligonucleotide
  source 1..20: mol_type = other DNA
accaagttcagtttccaggg

SEQ ID NO: 163 - DNA, 20 bp, synthetic construct
  misc_feature 1..20: note = Synthetic Antisense Oligonucleotide
  source 1..20: mol_type = other DNA
accaagttcagtttccaggg

SEQ ID NO: 164 - DNA, 20 bp, synthetic construct
  misc_feature 1..20: note = Synthetic Antisense Oligonucleotide
  source 1..20: mol_type = other DNA
ggattcaactgctgtccttg

SEQ ID NO: 165 - DNA, 20 bp, synthetic construct
  misc_feature 1..20: note = Synthetic Antisense Oligonucleotide
  source 1..20: mol_type = other DNA
ggattcaactgctgtccttg

SEQ ID NO: 166 - DNA, 20 bp, synthetic construct
  misc_feature 1..20: note = Synthetic Antisense Oligonucleotide
  source 1..20: mol_type = other DNA
atttcctccacaaccagctg

SEQ ID NO: 167 - DNA, 20 bp, synthetic construct
  misc_feature 1..20: note = Synthetic Antisense Oligonucleotide
  source 1..20: mol_type = other DNA
atttcctccacaaccagctg

SEQ ID NO: 168 - DNA, 20 bp, synthetic construct
  misc_feature 1..20: note = Synthetic Antisense Oligonucleotide
  source 1..20: mol_type = other DNA
atttcctccacaaccagctg

SEQ ID NO: 169 - DNA, 20 bp, synthetic construct
  misc_feature 1..20: note = Synthetic Antisense Oligonucleotide
  source 1..20: mol_type = other DNA
cagcctttttgtactgggac

SEQ ID NO: 170 - DNA, 20 bp, synthetic construct
  misc_feature 1..20: note = Synthetic Antisense Oligonucleotide
  source 1..20: mol_type = other DNA
cagcctttttgtactgggac

SEQ ID NO: 171 - DNA, 20 bp, synthetic construct
  misc_feature 1..20: note = Synthetic Antisense Oligonucleotide
  source 1..20: mol_type = other DNA
cagcctttttgtactgggac

SEQ ID NO: 172 - DNA, 20 bp, synthetic construct
  misc_feature 1..20: note = Synthetic Antisense Oligonucleotide
  source 1..20: mol_type = other DNA
gcttgctcctttcttggagg

SEQ ID NO: 173 - DNA, 20 bp, synthetic construct
  misc_feature 1..20: note = Synthetic Antisense Oligonucleotide
  source 1..20: mol_type = other DNA
gcttgctcctttcttggagg

SEQ ID NO: 174 - DNA, 20 bp, synthetic construct
  misc_feature 1..20: note = Synthetic Antisense Oligonucleotide
  source 1..20: mol_type = other DNA
gccatttccagatattcagg

SEQ ID NO: 175 - DNA, 20 bp, synthetic construct
  misc_feature 1..20: note = Synthetic Antisense Oligonucleotide
  source 1..20: mol_type = other DNA
gccatttccagatattcagg

SEQ ID NO: 176 - DNA, 20 bp, synthetic construct
  misc_feature 1..20: note = Synthetic Antisense Oligonucleotide
  source 1..20: mol_type = other DNA
gccatttccagatattcagg

SEQ ID NO: 177 - DNA, 20 bp, synthetic construct
  misc_feature 1..20: note = Synthetic Antisense Oligonucleotide
  source 1..20: mol_type = other DNA
ggccttcaacaatctctctt

SEQ ID NO: 178 - DNA, 20 bp, synthetic construct
  misc_feature 1..20: note = Synthetic Antisense Oligonucleotide
  source 1..20: mol_type = other DNA
gcctttttgtactgggacac

SEQ ID NO: 179 - DNA, 20 bp, synthetic construct
  misc_feature 1..20: note = Synthetic Antisense Oligonucleotide
  source 1..20: mol_type = other DNA
gcctttttgtactgggacac

SEQ ID NO: 180 - DNA, 20 bp, synthetic construct
  misc_feature 1..20: note = Synthetic Antisense Oligonucleotide
  source 1..20: mol_type = other DNA
gcctttttgtactgggacac

SEQ ID NO: 181 - DNA, 20 bp, synthetic construct
  misc_feature 1..20: note = Synthetic Antisense Oligonucleotide
  source 1..20: mol_type = other DNA
gactaccatttcatttggcc

SEQ ID NO: 182 - DNA, 20 bp, synthetic construct
  misc_feature 1..20: note = Synthetic Antisense Oligonucleotide
  source 1..20: mol_type = other DNA
gactaccatttcatttggcc

SEQ ID NO: 183 - DNA, 20 bp, synthetic construct
  misc_feature 1..20: note = Synthetic Antisense Oligonucleotide
  source 1..20: mol_type = other DNA
gactaccatttcatttggcc

SEQ ID NO: 184 - DNA, 20 bp, synthetic construct
  misc_feature 1..20: note = Synthetic Antisense Oligonucleotide
  source 1..20: mol_type = other DNA
tcatttccacagccctcagt

SEQ ID NO: 185 - DNA, 20 bp, synthetic construct
  misc_feature 1..20: note = Synthetic Antisense Oligonucleotide
  source 1..20: mol_type = other DNA
cctttcttggagggatgagg

SEQ ID NO: 186 - DNA, 20 bp, synthetic construct
  misc_feature 1..20: note = Synthetic Antisense Oligonucleotide
  source 1..20: mol_type = other DNA
ctgagcttgctcctttcttg

SEQ ID NO: 187 - DNA, 20 bp, synthetic construct
  misc_feature 1..20: note = Synthetic Antisense Oligonucleotide
  source 1..20: mol_type = other DNA
gcagctttttccttttcatc

SEQ ID NO: 188 - DNA, 20 bp, synthetic construct
  misc_feature 1..20: note = Synthetic Antisense Oligonucleotide
  source 1..20: mol_type = other DNA
cagcagcagaacatgcagct

SEQ ID NO: 189 - DNA, 20 bp, synthetic construct
  misc_feature 1..20: note = Synthetic Antisense Oligonucleotide
  source 1..20: mol_type = other DNA
tcttcttccatagcagcagc

SEQ ID NO: 190 - DNA, 20 bp, synthetic construct
  misc_feature 1..20: note = Synthetic Antisense Oligonucleotide
  source 1..20: mol_type = other DNA
gatgcttctgagtcttcttc

SEQ ID NO: 191 - DNA, 20 bp, synthetic construct
  misc_feature 1..20: note = Synthetic Antisense Oligonucleotide
  source 1..20: mol_type = other DNA
tcccctcccactacatttgc

SEQ ID NO: 192 - DNA, 20 bp, synthetic construct
  misc_feature 1..20: note = Synthetic Antisense Oligonucleotide
  source 1..20: mol_type = other DNA
tctgcaggattttccatagc

SEQ ID NO: 193 - DNA, 20 bp, synthetic construct
  misc_feature 1..20: note = Synthetic Antisense Oligonucleotide
  source 1..20: mol_type = other DNA
actgcttcttcaagtctgca

SEQ ID NO: 194 - DNA, 20 bp, synthetic construct
  misc_feature 1..20: note = Synthetic Antisense Oligonucleotide
  source 1..20: mol_type = other DNA
agtcttttctgttcatctgt

SEQ ID NO: 195 - DNA, 20 bp, synthetic construct
  misc_feature 1..20: note = Synthetic Antisense Oligonucleotide
  source 1..20: mol_type = other DNA
acaggtgctctgtctgtgcc

SEQ ID NO: 196 - DNA, 20 bp, synthetic construct
  misc_feature 1..20: note = Synthetic Antisense Oligonucleotide
  source 1..20: mol_type = other DNA
ctgtgtctgggccatttttg

SEQ ID NO: 197 - DNA, 20 bp, synthetic construct
  misc_feature 1..20: note = Synthetic Antisense Oligonucleotide
  source 1..20: mol_type = other DNA
acctttctgtgtctgggcca

SEQ ID NO: 198 - DNA, 20 bp, synthetic construct
  misc_feature 1..20: note = Synthetic Antisense Oligonucleotide
  source 1..20: mol_type = other DNA
gtaggtaacctttctgtgtc

SEQ ID NO: 199 - DNA, 20 bp, synthetic construct
  misc_feature 1..20: note = Synthetic Antisense Oligonucleotide
  source 1..20: mol_type = other DNA
acagcctttttgtactggga

SEQ ID NO: 200 - DNA, 20 bp, synthetic construct
  misc_feature 1..20: note = Synthetic Antisense Oligonucleotide
  source 1..20: mol_type = other DNA
tgaaatctgctgttccagcc

SEQ ID NO: 201 - DNA, 20 bp, synthetic construct
  misc_feature 1..20: note = Synthetic Antisense Oligonucleotide
  source 1..20: mol_type = other DNA
aggctcaacctcaagcagta

SEQ ID NO: 202 - DNA, 20 bp, synthetic construct
  misc_feature 1..20: note = Synthetic Antisense Oligonucleotide
  source 1..20: mol_type = other DNA
tccctgtcctttcatatact

SEQ ID NO: 203 - DNA, 20 bp, synthetic construct
  misc_feature 1..20: note = Synthetic Antisense Oligonucleotide
  source 1..20: mol_type = other DNA
gcactttccccagtaaactt

SEQ ID NO: 204 - DNA, 20 bp, synthetic construct
  misc_feature 1..20: note = Synthetic Antisense Oligonucleotide
  source 1..20: mol_type = other DNA
cctttcttggagggatgagg

SEQ ID NO: 205 - DNA, 20 bp, synthetic construct
  misc_feature 1..20: note = Synthetic Antisense Oligonucleotide
  source 1..20: mol_type = other DNA
cctttcttggagggatgagg

SEQ ID NO: 206 - DNA, 20 bp, synthetic construct
  misc_feature 1..20: note = Synthetic Antisense Oligonucleotide
  source 1..20: mol_type = other DNA
cctttcttggagggatgagg

SEQ ID NO: 207 - DNA, 20 bp, synthetic construct
  misc_feature 1..20: note = Synthetic Antisense Oligonucleotide
  source 1..20: mol_type = other DNA
acaggtgctctgtctgtgcc

SEQ ID NO: 208 - DNA, 20 bp, synthetic construct
  misc_feature 1..20: note = Synthetic Antisense Oligonucleotide
  source 1..20: mol_type = other DNA
acaggtgctctgtctgtgcc

SEQ ID NO: 209 - DNA, 20 bp, synthetic construct
  misc_feature 1..20: note = Synthetic Antisense Oligonucleotide
  source 1..20: mol_type = other DNA
acaggtgctctgtctgtgcc

SEQ ID NO: 210 - DNA, 20 bp, synthetic construct
  misc_feature 1..20: note = Synthetic Antisense Oligonucleotide
  source 1..20: mol_type = other DNA
acctttctgtgtctgggcca

SEQ ID NO: 211 - DNA, 20 bp, synthetic construct
  misc_feature 1..20: note = Synthetic Antisense Oligonucleotide
  source 1..20: mol_type = other DNA
acctttctgtgtctgggcca

SEQ ID NO: 212 - DNA, 20 bp, synthetic construct
  misc_feature 1..20: note = Synthetic Antisense Oligonucleotide
  source 1..20: mol_type = other DNA
acctttctgtgtctgggcca

SEQ ID NO: 213 - DNA, 20 bp, synthetic construct
  misc_feature 1..20: note = Synthetic Antisense Oligonucleotide
  source 1..20: mol_type = other DNA
acagcctttttgtactggga

SEQ ID NO: 214 - DNA, 20 bp, synthetic construct
  misc_feature 1..20: note = Synthetic Antisense Oligonucleotide
  source 1..20: mol_type = other DNA
acagcctttttgtactggga

SEQ ID NO: 215 - DNA, 20 bp, synthetic construct
  misc_feature 1..20: note = Synthetic Antisense Oligonucleotide
  source 1..20: mol_type = other DNA
acagcctttttgtactggga

SEQ ID NO: 216 - DNA, 20 bp, synthetic construct
  misc_feature 1..20: note = Synthetic Antisense Oligonucleotide
  source 1..20: mol_type = other DNA
gcactttccccagtaaactt

SEQ ID NO: 217 - DNA, 20 bp, synthetic construct
  misc_feature 1..20: note = Synthetic Antisense Oligonucleotide
  source 1..20: mol_type = other DNA
gcactttccccagtaaactt

SEQ ID NO: 218 - DNA, 20 bp, synthetic construct
  misc_feature 1..20: note = Synthetic Antisense Oligonucleotide
  source 1..20: mol_type = other DNA
gcactttccccagtaaactt

SEQ ID NO: 219 - DNA, 20 bp, synthetic construct
  misc_feature 1..20: note = Synthetic Antisense Oligonucleotide
  source 1..20: mol_type = other DNA
acagcctttttgtactggga

SEQ ID NO: 220 - DNA, 20 bp, synthetic construct
  source 1..20: mol_type = other DNA
  misc_feature 1..20: note = Synthetic Antisense Oligonucleotide
aaggctcaacctcaagcagt

SEQ ID NO: 221 - DNA, 20 bp, synthetic construct
  source 1..20: mol_type = other DNA
  misc_feature 1..20: note = Synthetic Antisense Oligonucleotide
ggctcaacctcaagcagtaa

SEQ ID NO: 222 - DNA, 20 bp, synthetic construct
  source 1..20: mol_type = other DNA
  misc_feature 1..20: note = Synthetic Antisense Oligonucleotide
gctcaacctcaagcagtaat

SEQ ID NO: 223 - DNA, 20 bp, synthetic construct
  source 1..20: mol_type = other DNA
  misc_feature 1..20: note = Synthetic Antisense Oligonucleotide
aatagccagacccagtacta

SEQ ID NO: 224 - DNA, 20 bp, synthetic construct
  source 1..20: mol_type = other DNA
  misc_feature 1..20: note = Synthetic Antisense Oligonucleotide
atagccagacccagtactat

SEQ ID NO: 225 - DNA, 20 bp, synthetic construct
  source 1..20: mol_type = other DNA
  misc_feature 1..20: note = Synthetic Antisense Oligonucleotide
cagacccagtactatgccaa

SEQ ID NO: 226 - DNA, 20 bp, synthetic construct
  source 1..20: mol_type = other DNA
  misc_feature 1..20: note = Synthetic Antisense Oligonucleotide
ccagtactatgccaatcaga

SEQ ID NO: 227 - DNA, 20 bp, synthetic construct
  source 1..20: mol_type = other DNA
  misc_feature 1..20: note = Synthetic Antisense Oligonucleotide
gtgcaggcttcatttccaca

SEQ ID NO: 228 - DNA, 20 bp, synthetic construct
  source 1..20: mol_type = other DNA
  misc_feature 1..20: note = Synthetic Antisense Oligonucleotide
ttccacagccctcagttaac